(19) United States
(12) Patent Application Publication
(10) Pub. No.: US 2022/0259622 A1
(43) Pub. Date: Aug. 18, 2022

(54) CONSTRUCTION METHOD AND RECOMBINANT YEAST STAIN YARROWIA LIPOLYTICA FOR XYLITOL SYNTHESIS

(71) Applicant: Shanghai Jiao Tong University, Shanghai (CN)

(72) Inventor: Hairong Cheng, Shanghai (CN)

(21) Appl. No.: 17/737,012

(22) Filed: May 4, 2022

Related U.S. Application Data
(63) Continuation of application No. PCT/CN2020/089747, filed on May 12, 2020.

(30) Foreign Application Priority Data
Nov. 14, 2019 (CN) 201911111632.4

(51) Int. Cl. C12P 7/18; C12N 1/16; C12N 15/52; C12N 15/81

(52) U.S. Cl. CPC C12P 7/18; C12N 1/16; C12N 15/52; C12N 2800/102; C12Y 101/01307; C12Y 101/01009; C12Y 106/01002; C12N 15/815

(57) ABSTRACT

The present invention discloses a construction method and a recombinant yeast stain Yarrowia lipolytica for xylitol synthesis; Adopting Yarrowia lipolytica as the host, introducing genes into the host through metabolic engineering to enable the recombinant yeast to synthesize xylitol from glucose, fructose, glycerol and starch as carbon sources, block the synthesis pathway of by-products, so that it can synthesize xylitol from the aforesaid carbon sources by fermentation, thus obtain the engineered Yarrowia lipolytica strain to synthesize xylitol from glucose and other carbon sources. After fermentation, xylitol crystal is obtained by ion exchange, decolorization, concentration and crystallization of the clear and transparent fermentation liquor after isolation of the strains from the fermentation. This construction method of engineered Yarrowia lipolytica described in the invention, and the Yarrowia lipolytica strain obtained by this method can simplify the existing method for chemical synthesis of xylitol and have good application.

### CROSS-REFERENCE TO RELATED APPLICATION

The present application is a Continuation Application of PCT Application No. PCT/CN2020/089747 filed on May 12, 2020, which claims priority from Chinese Patent Application No. 201911111632.4 filed on Nov. 14, 2019. The contents of the above are hereby incorporated by reference in their entirety.

### REFERENCE TO SEQUENCE LISTING

A sequence listing is submitted as an ASCII formatted text filed via EFS-Web, with a file name of “Sequence_listing.TXT”, a creation date of Apr. 29, 2022, and a size of 129,143 bytes. The sequence Listing filed via EFS-Web is part of the specification and is incorporated in its entirety by reference herein.

### TECHNICAL FIELD

This present invention belongs to the field of food biotechnology, relates to a construction method and a recombinant yeast stain Yarrowia lipolytica for synthesizing xylitol; Involves the following more specific constuction method of xylitol synthesis by means of metabolic engineering, gene engineering and synthetic biology through fermentation which adopts the Yarrowia lipolytica as the host microorganism, and using this method to obtain the recombinant Yarrowia lipolytica capable of synthesizing xylitol by fermentation with glucose and other carbon sources, then using the recombinant strain to synthesize xylitol by fermentation.

### BACKGROUND

Xylitol is a pentahydric alcohol, CAS number of 87-99-0, molecular weight of 152.15 dalton, is a common food additive, often used in the preparation of chewing gum, dairy products, candy and other foods to reduce the use of sucrose, which has a good effect of preventing oral diseases, reducing obesity and preventing diabetes. In addition to being widely used in food, it is widely used in medicine and chemical industry. Due to the wide application of xylitol, its market demand is also very large. According to incomplete statistics, the international market demand in 2018 is predicted to be more than 80,000 tons.

At present, the industrial production of xylitol still adopts the synthesis method of biomass hydrolysis combined with chemical hydrogenation, which requires complex steps such as acid hydrolysis of biomass, alkali neutralization, crystallization and redissolution of xylose, chemical hydrogen production and hydrogenation, etc. (e.g. Chinese invention patent: CN200910018483.7, Novel process for preparing xylitol; U.S. Pat. No. 4,066,711, Method for recovering xylitol; U.S. Pat. No. 3,586,537, Process for the production of xylose), and has disadvantages such as many steps, high pollution, high energy consumption and high risk factor. In recent years, there have been reports on the synthesis of xylitol by using xylose or directly using biomass hydrolysate as raw material through biological fermentation (e.g. US patent: US20040191881, Fermentation process for production of xylitol from Pichia sp; US20110003356, Process for production of xylitol; US20130217070, Production of xylitol from a mixture of hemicellulosic sugars; Chin et al., Analysis of NADPH supply during xylitol production by engineered Escherichia coli, Biotechnol. Bioeng., 2009, 102, 209-220), however, as the preparation of xylose or biomass hydrolysate still requires acid hydrolysis, alkali neutralization and xylose extraction, in addition, besides containing xylose, the hydrolysate also contains arabinose and other impurities, after biological fermentation and transformation, besides the target product xylitol, it also contains a lot of L-arabitol, which increases the difficulty to isolate the product and reduces the yield of xylitol. Moreover, the biomass acid hydrolysate contains inhibitors such as furfural, which can inhibit the growth and fermentation of microorganisms. Therefore, this approach, synthesizing xylitol directly from xylose or biomass hydrolysate by fermentation, is difficult to get practical application. Thus, it has important practical value to find other cheap and easy carbon sources to synthesize xylitol.

Glucose is a common, readily available and inexpensive carbon source, and is one of the most commonly used carbon sources for fermentation products. Therefore, it has important application value if glucose can be used as raw material to directly synthesize xylitol by fermentation of glucose through modified microorganisms. The first approach to synthesize xylitol from glucose is that the glucose is converted to the intermediate 5-p xylulose via pentose phosphate pathway, and then dephosphorylated to D-xylulose, or reduced to 1-p xylitol, and then dephosphorylated to xylitol. For example, Finnish scholar Mervi H. Toivari and others reported that by overexpressing XYL2 and DOG1 genes in modified Saccharomyces cerevisiae, to obtain the recombinant strain that can synthesize xylitol by glucose via fermentation. With glucose of 20 g/L as the carbon source, to obtain the xylitol with the highest concentration of 290 mg/L, in the meanwhile, fermentation medium also contains ribose of 440 mg/L and pentose such as D-ribose (Toivari et al., Metabolic engineering of Saccharomyces cerevisiae for conversion of D-glucose to xylitol and other five-carbon sugars and sugar alcohols, Appl. Environ. Microbiol., 2007, 73, 5471-5476). Povelainen and Miasnikov, also from Finland, reported that by overexpressing xylitol-phosphate dehydrogenase (XPDH) in Bacillus subtilis, recombinant Bacillus subtilis which can directly synthesis xylitol from glucose by fermentation can be obtained. After 300 hours of fermentation in the fermentation medium containing 100 g/L glucose, 23±1.8 g/L xylitol can be obtained. At the same time, ribitol, D-xylulose, D-ribulose and other by-products are also produced (Povelainen and Miasnikov, Production of xylitol by metabolically engineered strains of Bacillus subtilis, J. Biotechnol., 2007, 128, 24-31). The practical application of this method is limited by the long fermentation process, low yield and the need to add antibiotics.

Another approach to synthesize xylitol from glucose is that the glucose is firstly converted to D-arabinol, then converted to D-xylitol under the catalysis of D-arabinol-4-dehydrogenase, and then reduced to xylitol under the catalysis of xylitol dehydrogenase. In 2014, Cheng Hairong et al., the inventor of this application, reported that the recombinant strains of Pichia pastoris capable of synthesizing xylitol directly from glucose by fermentation was obtained by heterologous expression of arabinol dehydrogenase gene and xylitol dehydrogenase gene in the Pichia pastoris, by using the Pichia pastoris as the host microorganism. 15.2 g/L xylitol could be produced by fermentation of 220 g/L glucose, and the yield was 7.8% (Cheng et al., Genetically engineered Pichia pastoris yeast for conversion of glucose to xylitol by a single-fermentation process, Appl. Microbiol. Biotechnol., 2014, 98, 3539-3552). The low yield may be due to the low ability of Pichia pastoris itself to synthesize D-arabinol from glucose. It is possible to obtain a recombinant strain with high xylitol yield by using other osmophilic yeast with high D-arabinol synthesis ability. For example, the US invention patent US20170130209-A1 reported that the recombinant strains of Pichia ohmeri capable of synthesizing xylitol directly from glucose by fermentation was obtained also by heterologous expression of D-arabinol dehydrogenase and xylitol dehydrogenase genes in the osmophilic yeast to synthesize D-arabinol from glucose by fermentation, among which, the engineered stain encoding CNCM I-4981 could ferment 250 g/L monohydrate glucose to produce 120 g/L xylitol within 66 hours, and the yield reached 48%, which was the maximum output and yield reported in the known literature.

Although the recombinant Pichia ohmeri strain described in the above patent (US20170130209-A1) can synthesize more xylitol from glucose by fermentation, which has certain value of industrial practical use, however, glucose is first synthesized to 5-p-ribulose, through the oxidized pentose phosphate pathway, then dephosphorylated to ribulose, then reduced to D-arabinol, and then oxidized to D-xylitol under the catalysis of arabinol dehydrogenase, and then synthesize xylitol under the catalysis of xylitol dehydrogenase. The whole synthesis process requires the intermediate of D-arabitol, which increases the synthesis steps and consumes the energy in microbial cells. If D-xylolose can be directly generated from glucose through pentose phosphate oxidation pathway, and then reduced to xylitol, without the D-arabitol pathway, the efficiency of xylitol synthesis from glucose may be further improved. In addition, it is also important to select strains with high flux of pentose phosphate pathway to increase the amount of D-xylulose, an intermediate product of xylitol.

### SUMMARY

A main object of the present invention is to surmount the deficiency of the existing strains that synthesize xylitol by direct fermentation of glucose, to provide a construction method and a recombinant yeast strain Yarrowia lipolytica capable of synthesizing xylitol; Specifically, it is to design the method of engineering strain Yarrowia lipolytica capable of synthesizing xylitol directly by fermentation from glucose and other carbon sources, and use this method to construct the engineered strains of Yarrowia lipolytica which can efficiently synthesize xylitol, and use this strain to synthesize and purify xylitol through directly fermentation.

By means of metabolic engineering, genetic engineering and synthetic biology, this present invention genetically modifies Yarrowia lipolytica so that the Yarrowia lipolytica can synthesize xylitol from glucose and other carbon sources; More specifically, it adopts the Yarrowia lipolytica as the synthetic chassis, named as Yarrowia lipolytica, formerly known as Candida lipolytica. A method of utilizing gene editing to the Yarrowia lipolytica by means of metabolic engineering modification, introducing genes that synthesize xylitol from glucose, fructose, glycerol and starch as carbon sources, blocking the metabolic pathway of by-product synthesis, so that the recombinant Yarrowia lipolytica can synthesize xylitol from glucose, fructose, glycerol and starch as carbon sources by fermentation, thus obtain the engineered strains to synthesize xylitol from glucose and other carbon sources. And then, optimize from the constructed strains to obtain a strain of Yarrowia lipolytica ery959ΔTKLΔMDHΔArDHΔRPIΔXKS1 CGMCC No. 18479 with the highest ability to synthesize xylitol, and provide the method to synthesize and purify xylitol from glucose by fermentation.

The present invention is realized through the following technical scheme:

First, this present invention involves a method for constructing the recombinant Yarrowia lipolytica strains capable of synthesizing xylitol, a method that taking the Yarrowia lipolytica stains (formerly known as Candida lipolytica) as the host microorganisms to construct the recombinant Yarrowia lipolytica strains capable of synthesizing xylitol by fermentation with one carbon source or more carbon sources, including of glucose, fructose, glycerol and starch as carbon sources by means of metabolic engineering, genetic engineering and synthetic biology.

The Yarrowia lipolytica strains used in the present invention can be Yarrowia lipolytica strains commonly used in laboratories, which have low efficiency in synthesizing polyols such as mannitol or erythritol, e.g. Yarrowia lipolytica CLIB122 (Dujon et al., Genome evolution in Yeasts. Nature, 2004, 430(6995), 35-44.), Yarrowia lipolytica CLIB89/W29 (Magnan et al., Sequence Assembly of Yarrowia lipolytica Strain W29/CLIB89 Shows Transposable Element Diversity, PLoS One, 2016, 11(9), e0162363), Yarrowia lipolytica CLIB80, etc., and these strains can be obtained from the relevant strain deposit centers. By experiment research, CLIB122, CLIB89 and CLIB80 strains were cultivated in a medium of glucose 250 g/L at 30° C. (medium composition: anhydrous glucose 250 g/L, yeast cell powder 8 g/L, ammonium citrate 5 g/L, peptone 3 g/L, copper chloride 0.05 g/L, initial pH5.5), and shaking at 220 rpm. After 150 hours of fermentation, the content of erythritol is detected to be less than 15 g/L, mannitol is less than 20 g/L, and glucose residue is 160-180 g/L, indicating that these yeasts are not only inefficient in the synthesis of polyols, but also slow in glucose utilization.

As an embodiment of the present invention, the Yarrowia lipolytica host microorganism used in the invention can be other Yarrowia lipolytica strains containing DNA sequences with 97% or more homology or similarity to the SEQ ID NO. 3 sequence, such as CGMCC 7326 (Huiling Cheng et al. Characterization of two NADPH-dependent erythrose reductases in the yeast Yarrowia lipolytica and improvement of erythritol productivity using metabolic engineering. Microbial Cell Factories, 2018, 17:133.) etc.

As a specific embodiment of the present invention, the Yarrowia lipolytica used in the invention can also be Yarrowia lipolytica stain ery929 CGMCC No. 18478, which is highly efficient in synthesizing erythritol. After molecular identification, it is identified as Yarrowia lipolytica, whose 26S rDNA sequence (SEQ ID NO. 3 sequence) is 98% or higher identity with the 26S rDNA of Yarrowia lipolytica in known databases (e.g. Yarrowia lipolytica 26S rDNA sequence in NCBI database).

Scheme 1: The construction method of recombinant Yarrowia lipolytica strain capable of synthesizing xylitol in this present invention including the expression of one or more of the following genes cell of the Yarrowia lipolytica (to obtain corresponding functions):

(1) the gene encoding xylitol dehydrogenase (also known as xylulose reductase); (gaining the ability to reducing xylulose to xylitol)

(2) the gene encoding 5-p xylitol dehydrogenase (also known as 5-p xylulose reductase); (gaining the ability to reducing 5-p xylulose to 5-p xylitol)

(3) the gene encoding 5-p xylulose phosphatase; (gaining the ability to dephosphorylate 5-p xylulose phosphatase into xylulose)

(4) the gene encoding xylitol transporter; (gaining the ability to transport xylitol to medium)

(5) the gene encoding NADP transhydrogenase. (gaining the ability to transform NADH to NADPH and vice versa)

Scheme 2: The construction method of recombinant Yarrowia lipolytica strain capable of synthesizing xylitol in this present invention including the knockout or down-regulation of one or more of the following genes in the cell of Yarrowia lipolytica (causing Yarrowia lipolytica to lose or decrease the corresponding function):

(1) mannitol dehydrogenase(MDH) gene; (Knockout and disrupt the mannitol dehydrogenase gene to make the recombinant strain lose the ability to synthesize mannitol, so as to improve the synthesis efficiency of xylitol)

(2) arabinitol dehydrogenase(ArDH) gene; (Knockout and disrupt the arabitol dehydrogenase gene to make the recombinant strain lose the ability to synthesize arabinitol, so as to improve the synthesis efficiency of xylitol)

(3) transketolase(TKL) gene; (Knockout, disrupt or down-regulate the transketolase gene to make the recombinant strain lose or significantly decrease the ability to synthesize erythritol, so as to improve the synthesis efficiency of xylitol)

(4) xylulose kinase(XKS) gene; (Knockout and disrupt the xylulose kinase gene to make the recombinant strain lose the ability to use the xylulose, so as to improve the synthesis efficiency of xylitol)

(5) 5-p ribulose isomerase(RPI) gene. (Knockout and disrupt the 5-p ribulose isomerase gene to make the recombinant strain lose the ability to synthesize 5-p ribose and increase the content of 5-p xyulose, so as to improve the synthesis efficiency of xylitol)

Scheme 3: The construction method of recombinant Yarrowia lipolytica strain capable of synthesizing xylitol by fermentation in this present invention including the expression of one or more of the following genes in Yarrowia lipolytica:

(1) the gene encoding xylitol dehydrogenase (also known as xylulose reductase);

(2) the gene encoding 5-p xylitol dehydrogenase (also known as 5-p xylulose reductase);

(3) the gene encoding 5-p xylulose phosphatase;

(4) the gene encoding xylitol transporter;

(5) the gene encoding NADP transhydrogenase;

Meanwhile, knockout, disrupt or down-regulate one or more of the following genes from its own genome:

(6) mannitol dehydrogenase(MDH) gene;

(7) arabinitol dehydrogenase(ArDH) gene;

(8) transketolase(TKL) gene;

(9) xylulose kinase(XKS) gene;

(10) 5-p ribulose isomerase(RPI) gene.

Specifically, the modification to Yarrowia lipolytica by means of metabolic engineering, genetic engineering and synthetic biology, enabling the recombinant Yarrowia lipolytica capable of synthesizing xylitol efficiently from glucose, is implemented through the following methods:

(1) Using Yarrowia lipolytica as the host microorganism. Any strain of Yarrowia lipolytica or Candida lipolytica, including Yarrowia lipolytica CLIB122, Yarrowia lipolytica CLIB89/W29, Yarrowia lipolytica CLIB80, Yarrowia lipolytica ery929 CGMCC No.18478 and CGMCC No.7326 mentioned above, belong to the scope of the host microorganism used in the present invention. One of the characteristics of the strains of Yarrowia lipolytica host microorganism used in the present invention is that its genome contains DNA sequences with 97% or higher homology or similarity with SEQ ID NO. 3 sequence.

(2) Optimally synthesize the xylitol dehydrogenase gene (also known as xylulose reductase gene) according to the codon bias of Yarrowia lipolytica, and expression in the cell of Yarrowia lipolytica.

Xylitol dehydrogenase gene is cloned from, but not limited to, the following microorganisms: Scheffersomyces stipitis (also known as Pichia stiptis, SEQ ID NO. 4), Debaryomyces Hansenii (SEQ ID NO. 5), Agrobacterium sp. (SEQ ID NO. 6), Gluconobacter oxydans (SEQ ID NO. 7, SEQ ID NO. 8), Candida maltosa (SEQ ID NO. 9), Trichoderma reesei (SEQ ID NO. 10), Neurospora crassa (SEQ ID NO. 11), Saccharomyces cerevisiae (SEQ ID NO. 12), or the xylitol dehydrogenase gene (SEQ ID NO. 13) of Yarrowia lipolytica. Preferably, use the xylitol dehydrogenase genes of Scheffersomyces stipitis, Debaryomyces hansenii, Gluconobacter oxydans, Candida maltosa and Yarrowia lipolytica. More preferably, use the xylitol dehydrogenase genes of Gluconobacter oxydans and Candida maltosa.

(3) While expressing the xylitol dehydrogenase gene in the cell of Yarrowia lipolytica, the gene encoding 5-p xylitol dehydrogenase (also known as 5-p xylulose reductase) can also be expressed.

These genes can be optimally synthesized according to the codon bias of Yarrowia lipolytica, the enzyme expressed can reduce 5-p xylulose, an intermediate in the pentose phosphate pathway, to 5-p xylitol. This gene is cloned from but not limited to the following microorganisms: Clostridioides difficile (SEQ ID NO. 14; SEQ ID NO. 15; SEQ ID NO. 16), Lactobacillus rhamnosus (SEQ ID NO. 17), Lactobacillus paracasei (SEQ ID NO. 18), Lactobacillus casei (SEQ ID NO. 19), Lactobacillus plantarum (SEQ ID NO. 20). Preferably, use the 5-p xylitol dehydrogenase gene of Clostridioides difficile, Lactobacillus rhamnosus and Lactobacillus plantarum. More preferably, use the 5-p xylitol dehydrogenase gene of Clostridioides difficile and Lactobacillus rhamnosus.

(4) Other genes that encode 5-p xylulose phosphatase activity can also be expressed in the cell of Yarrowia lipolytica.

The 5-p xylulose phosphatase can dephosphorylate 5-p xylulose to xylulose, which can be converted to xylitol under the catalysis of xylitol dehydrogenase or xylulose reductase. Therefore, enhancing the activity of 5-p xylulose phosphatase in Yarrowia lipolytica can improve the level of intracellular xylulose, and then improve the conversion level of xylitol. These genes can be optimally synthesized according to the codon bias of Yarrowia lipolytica. This gene is cloned from but not limited to the following microorganisms: Kluyveromyces marxianus (SEQ ID NO. 21), Saccharomyces cerevisiae (SEQ ID NO. 22; SEQ ID NO.23), Komagataella phaffii (SEQ ID NO. 24), Lactobacillus kunkeei (SEQ ID NO. 25), Lactobacillus paracasei (SEQ ID NO. 26), Lactobacillus plantarum (SEQ ID NO. 27), Lactobacillus fermentum (SEQ ID NO. 28), Aspergillus niger (SEQ ID NO. 29), Aspergillus japonicus (SEQ ID NO. 30), Bacillus subtilis (SEQ ID NO. 31). Preferably, use the 5-p xylulose phosphatase gene of Kluyveromyces marxianus, Saccharomyces cerevisiae, Komagataella phaffii, Lactobacillus plantarum and Bacillus subtilis. More preferably, use the 5-p xylulose phosphatase gene of Kluyveromyces marxianus and Bacillus subtilis. Most preferably, use the 5-p xylulose phosphatase gene of Bacillus subtilis.

(5) Xylitol transporter gene can also be expressed in the cell of Yarrowia lipolytica.

After xylitol is synthesized in the cell, it needs to be transported to the extracellular quickly to reduce the feedback inhibition on enzymes by the accumulation of xylitol in the cell. Therefore, the present invention is to express the xylitol transporter gene in Yarrowia lipolytica cells, and the encoding product xylitol transporter can transport xylitol to the extracellular, thus reducing feedback inhibition to further improve the efficiency of xylitol synthesis with intracellular enzymes. These genes can be optimally synthesized according to the codon bias of Yarrowia lipolytica. This gene is cloned from but not limited to the following microorganisms: Saccharomyces cerevisiae (SEQ ID NO. 32), Kluyveromyces marxianus (SEQ ID NO. 33), Torulaspora delbrueckii (SEQ ID NO. 34), Candida glabrata strain DSY562 (SEQ ID NO. 35), Zygosaccharomyces parabailii (SEQ ID NO. 36), Zygosaccharomyces rouxii (SEQ ID NO. 37), Kluyveromyces lactis (SEQ ID NO. 38), and can also be cloned from the xylitol transporter gene of Yarrowia lipolytica itself (SEQ ID NO. 39; SEQ ID NO. 40). Preferably, use the xylitol transporter gene of Saccharomyces cerevisiae, Kluyveromyces marxianus, Zygosaccharomyces rouxii or Yarrowia lipolytica. More preferably, use the xylitol transporter gene of Saccharomyces cerevisiae, Kluyveromyces marxianus, or Yarrowia lipolytica. Most preferably, use the xylitol transporter gene of Yarrowia lipolytica.

(6) NADP transhydrogenase gene can also be expressed in the cell of Yarrowia lipolytica.

The engineered Yarrowia lipolytica losses or decreases its ability to synthesize erythritol or mannitol, both of which are synthesized by NADPH as a cofactor. Therefore, the level of NADPH in cells may increase after glucose is converted into xylulose through pentose phosphate pathway, while the synthesis of xylitol taking NADH as a cofactor, thus NADPH transhydrogenase is introduced into Yarrowia lipolytica in order to achieve the balance between NADPH and NADH, then when NADPH is excessive, NADPH is transformed to NADH to provide enough cofactors for the synthesis of xylitol. The NADPH transhydrogenase gene can be optimally synthesized according to Yarrowia lipolytica codon biasis, is cloned from but not limited to the transhydrogenase gene of the following microorganisms: Azotobacter vinelandii (SEQ ID NO. 41), Escherichia coli str. K-12 (SEQ ID NO. 42), Aspergillus oryzae (SEQ ID NO. 43), Gluconobacter oxydans (SEQ ID NO. 44) and Bifidobacterium breve (SEQ ID NO. 45). Preferably, use the transhydrogenase gene of Aspergillus oryza or Bifidobacterium breve. Most preferably, use the transhydrogenase gene of Aspergillus oryzae.

The above genes related to xylitol synthesis are overexpressed in Yarrowia lipolytica in the following ways, which are only examples of how the target gene is integrated into Yarrowia lipolytica cells and are not a limitation to the present invention.

(1) Optimum Synthesis and Cloning of Gene.

Optimally synthesize the aforesaid genes that require enhanced expression and clone into integrative expression plasmid vector according to the codon bias of Yarrowia lipolytica. The integrative expression vector contains necessary DNA elements such as homologous integrative sequence (including left and right segments), promoter sequence, terminator sequence, autonomously repliacting sequence and selective marker sequence. There are multiclonal enzyme cutting sites between promoter and terminator sequences, which can clone the synthesized gene between promoter and terminator. The homologous integrative sequence in the present invention is a DNA sequence cloned from the genome of Yarrowia lipolytica, which can insert the DNA sequences between the left and right homologous arms into the homologous DNA sequences in the genome through the method of double crossover homologous recombination. The promoter is a sequence of DNA capable of inducing the transcription of its downstream genes. This sequence can be a synthetic promoter sequence such as UAS1B8, UAS1B16, hp4d, etc. (Blazeck et al. 2013. Generalizing a hybrid synthetic promoter approach in Yarrowia lipolytica. Appl Microbiol Biotechnol, 97:3037-3052.), or the gene sequences from Yarrowia lipolytica itself, such as promoter sequence of erythritose reductase gene and promoter sequence of 3-p glycerol dehydrogenase gene The terminator is a sequence of DNA capable of terminating its upstream genes for further transcription. The autonomously repliacting sequence in the present invention refers to the DNA sequence that can be replicated in the cell of prokaryotic bacteria such as Escherichia coli or eukaryotic fungi such as Yarrowia lipolytica. The inclusion of this sequence enables the integrative expression plasmid vector to replicate and amplify autonomously in both prokaryotic bacteria such as Escherichia coli and eukaryotic fungi such as Yarrowia lipolytica. The selective marker sequence refers to the antibiotics resistance genes such as ampicillin resistance genes, or nutrition selective genes such as sucrase gene (Suc2, the encoding product enables Yarrowia lipolytica to use sucrose), xylitol dehydrogenase (XDH, the encoding product enables Yarrowia lipolytica to use xylitol), uracil monophosphate synthetase gene 3 (URA3, the encoding product enables URA3 defect Yarrowia lipolytica to grow on uracil free minimal medium). etc. Typical integrative expression plasmid vectors are shown in FIG. 2: The plasmid contains necessary DNA elements such as left and right homologous integrative sequence, promoter sequence, target gene sequence, terminator sequence, selective marker sequence of Yarrowia lipolytica, autonomously repliacting sequence of Yarrowia lipolytica (e.g. ARS18, etc.), replication origin sequence of bacteria (e.g. ori sequence) and selective marker sequence of bacteria. For the aforesaid necessary DNA elements, besides the above target gene sequences (e.g. xylitol dehydrogenase gene, 5-p xylulose phosphatase gene, etc.) used in the present invention, the rest can be obtained in public databases (such as database: https://www.ncbi.nlm.nih.gov/).

(2) Transformation of Integrated Expression Vector Containing Target Gene.

Linearize the integrated expression vector containing target gene with the restriction enzyme (e.g. NotI, EcoRI, etc.), transform to Yarrowia lipolytica (For the specific transformation method, please refer to the paper published by the inventor Cheng Hairong: Journal of Functional Foods, 2017, 32:208 ˜217), then screen in medium containing selective markers. If the integrated expression vector contains the sucrase selective markers, the yeast should be spread on YNB minimal medium containing sucrose for screening after transformation (6 g/L of yeast nitrogen base, 5 g/L of ammonium sulfate, 10 g/L of sucrose, 15 g/L of agar powder, pH 6.0). If the integrated expression vector contains the hygromycin resistance gene selective markers, the yeast should be spread on YPD culture medium for screening after transformation (10 g/L of glucose, 10 g/L of yeast cell powder, 5 g/L of peptone, 15 g/L of agar, 300 μg/ml of hygromycin, pH 6.0). Extract the genome of the transformant, use a pair of primers on the target gene for amplification. If the corresponding size of the band can be amplified and the sequencing is correct, it indicates that the target gene has been integrated into the genome of Yarrowia lipolytica. Then, use the Cre/1oxP system to recover the selective markers in the transformant (reference: J. Microbiol. Methods, 2003, 55, 727-737). For specific recovery and screening methods, please refer to the embodiments. After the first target gene is integrated into the genome, the engineered strain obtained after the recovery of selective markers can be used as the host to continue the transformation of the second target gene. After the verification of the integration of the second target gene and the recovery of the selective marker, the new engineered strain obtained therein can be used as the host for the transformation of other target genes, operate successively until all the target genes are integrated into the genome and the selective marker genes are removed. And finally obtain the Yarrowia lipolytica ery959 containing the abovementioned genes related to xylitol synthesis, including (1) xylitol dehydrogenase gene, (2) 5-p xylitol dehydrogenase gene, (3) 5-p xylulose phosphatase gene, (4) xylitol transporter gene and (5) NADP transhydrogenase gene.

Afterwards, process further modification to Yarrowia lipolytica by means of metabolic engineering, genetic engineering and synthetic biology to enable the recombinant Yarrowia lipolytica capable of synthesizing xylitol from glucose. In addition to expressing the genes related to xylitol synthesis in Yarrowia lipolytica, the following genes are also knocked out or down-regulated to block or reduce by-product synthesis, making the effect of xylitol synthesis more significant.

(1) Knocking out of Mannitol Dehydrogenase Gene(YlMDH)

Two mannitol dehydrogenase genes, YlMDH1 (SEQ ID NO. 70) and YlMDH2 (SEQ ID NO. 71), are identified from the genome of Yarrowia lipolytica by comparing their protein sequences. After the determination of their activity by prokaryotic protein expression, both of these two mannitol dehydrogenases can synthesize mannitol with fructose as substrate, while mannitol synthesis competes substrate glucose with that of xylitol synthesis. Therefore, knocking out the mannitol dehydrogenase gene can theoretically improve the yield of xylitol synthesis.

(2) Knocking out of Arabitol Dehydrogenase Gene(YlArDH)

Two arabitol dehydrogenase genes, YlArDH1 (SEQ ID NO. 72) and YlArDH2 (SEQ ID NO. 73), are identified from the genome of Yarrowia lipolytica by comparing their protein sequences with other arabitol dehyfrogenase genes sequences. After identification of their activity by prokaryotic protein expression, both of these two dehydrogenases can synthesize arabitol with xylulose as substrate. Since both arabitol and xylitol are synthesized from glucose, knockout arabitol dehydrogenase gene can theoretically improve the yield of xylitol synthesis.

(3) Knocking out or Weak Expression (Down-Regulation of Gene Function) of Transketolase Gene(YlTKL)

Through genome function mining, the inventor found that Yarrowia lipolytica contains two transketolase genes, one of which is responsible for converting 5-p ribose and 5-p xylulose to 3-p glyceraldehyde and 7-p sedoheptulose with transketolase. The enzyme is transketolase 1 (encoded by YlTKL1 gene, SEQ ID NO. 74). Another one is responsible for converting 3-p glyceraldehyde and 6-p fructose to 5-p xylulose and 4-p erythrose with transketolase. The enzyme is transketolase 2 (encoded by YlTKL2 gene, SEQ ID NO. 75). Therefore, in order to eliminate or reduce the synthesis of erythritol, it is necessary to block or weaken the ketotransferase reaction, knock out or weaken the function of the two transketolase genes.

(4) Knocking out of Xylulokinase Gene(XKS1)

Through genome function mining, the inventor found that Yarrowia lipolytica contains the xylulose kinase gene (SEQ ID NO. 76), the encoding product xylulose kinase can phosphorylate xylulose to 5-p xylulose, and consume ATP at the same time. Since the xylulose is the immediate precursor to synthesize xylitol, phosphorylation of xylulose reduces the content of substrate xylulose, thus reducing the efficiency of xylitol synthesis and consuming ATP. Therefore, knocking out of XKS1 gene can theoretically improve the efficiency of xylitol synthesis and reduce the consumption of ATP.

(5) Knocking out of 5-p Ribulose Isomerase Gene(RPI Gene)

Through genome function mining, the inventor found that Yarrowia lipolytica contains the 5-p ribulose isomerase gene(RPI gene, SEQ ID NO. 77), the encoding product 5-p ribulose isomerase can isomerize the 5-p ribulose to 5-p ribose. Since the substrate of 5-p ribulose isomerase and 5-p ribulose epimerase (RPE) are 5-p ribulose, knocking out the 5-p ribulose isomerase gene can theoretically increase the flow of 5-p ribulose to 5-p xylulose, yet the 5-p ribulose is converted to xylulose under the catalysis of 5-p xylulose phosphorylase, then xylitol is synthsized under the catalysis of xylitol dehydrogenase. Therefore, knocking out the 5-p ribulose isomerase gene can theoretically increase xylitol synthesis.

In the second place, the present invention also involves a recombinant Yarrowia lipolytica stain which can synthesize xylitol from glucose and other carbon sources by using the construction method of recombinant Yarrowia lipolytica strain capable of synthesizing xylitol that is mentioned above.

A series of mutant strains of Yarrowia lipolytica are obtained through the above molecular biological operation, including strains overexpressing xylitol dehydrogenase gene (also known as xylulose reductase gene), 5-p xylitol dehydrogenase gene (also known as xylulose reductase gene), 5-p xylulose phosphatase gene, xylitol transporter gene and NADP transhydrogenase gene, and in the meanwhile, knocking out of mannitol dehydrogenase gene and arabinitol dehydrogenase gene, knocking out or weakening expression of transketolase gene, knocking out of xylulose kinase gene and 5-p ribulose isomerase gene. Test the strains obtained for xylitol synthesis by fermentation, select the representative strains with the best synthetic yield for deposition, and deposited as Yarrowia lipolytica CGMCC No. 18479.

7. Therefore, the recombinant Yarrowia lipolytica strain capable of synthesizing xylitol contructed by the present invention is preferably Yarrowia lipolytica ery959 ΔTKLΔMDHΔArDHΔRPIΔXKS1 CGMCC No. 18479. The strain is a Yarrowia lipolytica strain with the highest yield of xylitol synthesis obtained by fermentation, optimization and screening of all different recombinant strains constructed by the method of the present invention.

In the third place, the present invention also involves the method of fermentation for xylitol synthesis using a recombinant Yarrowia lipolytica strain capable of synthesizing xylitol; the said method includes the following steps:

S1. Culture the Yarrowia lipolytica strains in medium containing carbon, nitrogen, inorganic salts, amino acid and water, and oscillating or stirring, fermentation and culture at initial pH value of 3.0 ˜7.0 and temperature of 25 ˜35° C., then isolate the strains from the broth after fermentation to obtain xylitol-containing fermentation broth and yeast cells;

S2. Proceed isolation and purification of the xylitol-containing fermentation broth and yeast cells to obtain xylitol.

In the above step S1, during fermentation culture, take samples at intervals to detect the residual amount of substrate carbon source and the production amount of xylitol, and terminate fermentation when the substrate carbon source is used up.

In the above step S1, the carbon source in the said medium can be one or more of glucose, fructose, glycerol, starch, and the dosage of carbon source is 50-350 g/L.

In the above step S1, the nitrogen source in the said medium can be one of or a mixture of any combination of peptone, yeast cell powder, yeast extract, steep powder, diammonium phosphate, ammonium citrate, and amino acids. The nitrogen source content in the said medium can be 5 ˜20 g/L.

In the above step S1, the inorganic salt in the said medium is one or more of magnesium sulfate, manganese chloride, copper chloride, and zinc chloride. The inorganic salt content in the said medium can be 0 ˜0.44 g/L. The preferred dosage is 0.01 ˜0.44 g/L.

In the above step S2, the isolation and purification includes: the isolation of strains from the broth to obtain the clear fermentation broth containing xylitol, the concentration to obtain the concentrated solution rich in xylitol, the primary crystallization to obtain crude products of xylitol, which would obtain the refined products of xylitol through redissolution, ion exchange removal of ions, decolorization, concentration and secondary crystallization to the crude products, as well as the drying procedure.

The isolation of strains from the broth is: centrifugation or membrane filtration of the fermentation broth to separate and remove the yeast cells, washing cells twice to fully recover the xylitol, thus obtain the clear fermentation broth containing xylitol.

To sum up, in order to further optimize the pathway of synthesizing xylitol from glucose, the present invention selects Yarrowia lipolytica with high efficiency of pentose phosphate pathway as the original strain. The present invention provides a method of using a recombinant strain to synthesize and purify xylitol by fermentation from glucose and other carbon sources. The recombinant strain is Yarrowia lipolytica with the maximum production and the highest yield of xylitol, which is obtained through metabolic engineering, genetic engineering and synthetic biology to knock out or weaken express the genes associated with byproducts synthesis, and introduce the genes related to xylitol synthesis, develop the method to construct the recombinant Yarrowia lipolytica capable of synthesizing xylitol by direct fermentation from glucose and other carbon sources, as well as screening and optimization.

Yarrowia lipolytica ery929 of the present invention has been submitted for deposition at China General Microbiological Culture Collection Center (CGMCC) on Sep. 10, 2019, with the deposit number of CGMCC No. 18478, at the Institute of Microbiology, Chinese Academy of Sciences, addressed at No. 1, Beichen West Road, Chaoyang District, Beijing.

Yarrowia lipolytica ery959 ΔTKLΔMDHΔArDHΔRPIΔXKS1 of the present invention has been submitted for deposition at China General Microbiological Culture Collection Center (CGMCC) on Sep. 10, 2019, with the deposit number of CGMCC No. 18479, at the Institute of Microbiology, Chinese Academy of Sciences, addressed at No. 1, Beichen West Road, Chaoyang District, Beijing.

Compared with the prior art, the present invention has the following beneficial effects:

1) Xylitol is synthesized by direct fermentation from cheap and readily available carbon sources such as glucose, fructose and starch, avoiding the complicated steps of chemical synthesis of xylitol; Chemical synthesis requires the use of biomass such as corn cob for acid hydrolysis and chemical hydrogenation, which requires harsh conditions of high temperature and high pressure and the use of dangerous flammable and explosive hydrogen. On the contrary, the method of the present invention is green and safe by fermentation and synthesis under normal temperature and pressure.

2) The recombinant strain constructed by using the method designed by the present invention can directly synthesize xylitol from glucose, and the highest conversion rate is 50.7%, which basically has application value.

### BRIEF DESCRIPTION OF THE DRAWINGS

Other features, objectives and advantages of the present invention will become more apparent by reading the detailed description of non-restrictive embodiments with reference to the following attached drawings:

FIG. 1 is the schematic diagram of the polyol synthesized by yeast identified and screened by HPLC and GC-MS as erythritol. Among them, A: Identified by HPLC, the peak times of the two are consistent (in A of FIG. 1, 1 is the standard peak of erythritol, and 2 is the peak of the fermentation broth of the selected yeast). B: Standard mass spectrogram of erythritol; C: Mass spectrometry of polyols produced by fermentation of selected yeast; D: Combined comparison of B and C;

FIG. 2 shows a typical integrative expression plasmid of Yarrowia lipolytica;

FIG. 3 shows the integratived expression vector of xylitol dehydrogenase gene;

FIG. 4 shows the amplification curves of three of the five exogenous genes in the recombinant strain ery929. Where, A is the amplification curve of xylitol dehydrogenase gene; B is the amplification curve of 5-p xylulose reductase gene; C is the amplification curve of 5-p xylulose phosphatase gene;

FIG. 5 shows the amplification curves of two of the five exogenous genes in the recombinant strain ery929. Where, A is the amplification curve of xylitol transporter gene; B is the amplification curve of NADP transhydrogenase gene;

FIG. 6 shows electrophoresis verification after transketolase genes 1 and 2 are knocked out. Where, M: DNA molecular weight standards; lane 1: electrophoresis verification of YlTKL1 gene contrast to ery929 strain; lane 2: electrophoresis verification of YlTKL2 gene contrast to ery929 strain; lane 3: electrophoresis verification of YlTKL1 gene after YlTKL1 gene is knocked out in the mutant; lane 4: electrophoresis verification of YlTKL2 gene after YlTKL2 gene is knocked out in the mutant;

FIG. 7 shows electrophoresis verification after mannitol dehydrogenase genes 1 and 2 are knocked out. Where, lane 1: electrophoresis verification of YlMDH1 gene after YlMDH1 gene is knocked out in the mutant 1; lane 2: electrophoresis verification of YlMDH2 gene after YlMDH2 gene is knocked out in the mutant 1; lane 3: electrophoresis verification of YlMDH1 gene after YlMDH1 gene is knocked out in the mutant 2; lane 4: electrophoresis verification of YlMDH2 gene after YlMDH2 gene is knocked out in the mutant 2; M: DNA molecular weight standards; lane 5: electrophoresis verification of YlMDH1 gene contrast to ery929 strain; lane 6: electrophoresis verification of YlMDH2 gene contrast to ery929 strain;

FIG. 8 shows electrophoresis verification after arabinitol dehydrogenase genes 1 and 2 are knocked out. Where, M: DNA molecular weight standards; lane 1: electrophoresis verification of YlArDH1 gene contrast to ery929 strain; lane 2: electrophoresis verification of YlArDH2 gene contrast to ery929 strain; lane 3: electrophoresis verification of YlArDH1 gene after YlArDH1 gene is knocked out in the mutant; lane 4: electrophoresis verification of YlArDH2 gene after YlArDH2 gene is knocked out in the mutant;

FIG. 9 shows electrophoresis verification after 5-p ribulose isomerase gene (RPI) is knocked out; where, M: DNA molecular weight standards; lane 1-2: electrophoresis verification of RPI gene after RPI gene is knocked out in the mutant; lane 3: electrophoresis verification of RPI gene contrast to ery929 strain;

FIG. 10 shows electrophoresis verification after xylulose kinase gene (XKS1) is knocked out; where, M: DNA molecular weight standards; lane 1: electrophoresis verification of YlXKS1 gene contrast to YlXKS1 strain; lane 2: electrophoresis verification of YlXKS1 gene after YlXKS1 gene is knocked out in the mutant;

FIG. 11 shows the ion fragment peak of xylitol and standard xylitol synthesized by strain CGMCC 18479 of the present invention from glucose through fermentation and the comparison between the two. Among them, A: the ion fragment peak of xylitol synthesized by strain CGMCC 18479 from glucose through fermentation; B: ion fragment peak of standard xylitol; C: the comparison between the two.

### DETAILED DESCRIPTION

The present invention is hereby described in detail in combination with exemplary embodiments below. The following exemplary embodiments will help those skilled in the art to further understand the present invention, but shall not limit the present invention in any way. It should be noted that a number of adjustments and improvements can be made for those of ordinary skilled in the art without deviating from the concept of the present invention. These belong to the scope of protection of the present invention.

### Embodiment 1 Acquisition of Yarrowia lipolytica ey929 (CGMCC No. 18478)

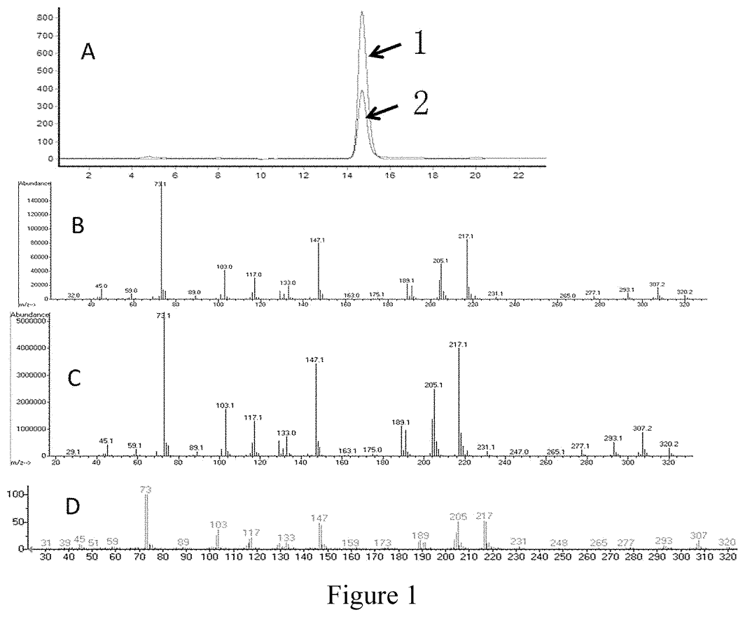

Take fresh bee hives from different sources and divide into several portions with 5 grams each. Use sterilized scissors to cut small pieces of each hive with the length less than 5 mm, soak them in 20 ml of sterile water containing 0.05% Tween 40, stir for 1 hour, centrifuge at 5000 rpm for 10 minutes, discard supernatant and hive fragments, suspend the precipitate with 1 ml of sterile water, and spread in sterilized hyperosmotic solid medium (composition: 400 g/L of anhydrous glucose, 12 g/L of yeast cell powder, 5 g/L of ammonium citrate, 3 g/L of peptone, 15 g/L of agar, pH5.5), spread 200 μl on each plate, then culture at 30° C. for 7 days. Select the yeast-like colony for pure culture, then take the pure cultured yeast colony and conduct the test of erythritol synthesis by fermentation. Liquid medium composition: 300 g/L of anhydrous glucose, 8 g/L of yeast cell powder, 5 g/L of ammonium citrate, 3 g/L of peptone, 0.02 g/L of copper chloride, 0.02 g/L of manganese chloride, 0.05 g/L of vitamin B1, initial pH5.5. After fermentation for 5 days in a 30° C. incubator shaker, use the HPLC to detect the fermentation broth and compare it with the standard erythritol. If the peak time is completely consistent with the standard erythritol, use the GC-MS to further detect the fermentation broth. HPLC and GC-MS results (FIG. 1) show that there is one strain produces the polyol of erythritol. After fermentation test, it can completely consume glucose from 300 g/L of glucose within 115 hours, and synthesize 162 g/L of erythritol, which is the wild-type yeast with the highest ability of erythritol synthesis among all the selected yeasts. Select the strain with the highest capacity to produce erythritol for 26S rDNA molecular identification. Extract the genome and use a pair of primers of 26S rDNA for PCR molecular identification. The pair of primers used for molecular identification are as follows:

P_{26srDNA-F}:
(SEQ ID NO. 1)
5′-tagtgcagatcttggtggtagtagc-3′
P_{26srDNA-R}:
(SEQ ID NO. 2)
5′-ctgcttcggtatgataggaagagc-3′

The amplification conditions are as follows:

(1) Initial denaturation at 95° C. for 5 minutes

(2) Denaturation at 94° C. for 30 seconds

(3) Annealing at 55° C. for 30 seconds

(4) Elongation at 72° C. for 90 seconds

(5) Final elongation at 72° C. for 10 minutes

Step (2) to step (4) shall perform 30 cycles.

According to the above conditions, take the genome of the yeast with the highest erythritol production as template for PCR, 1.4 kb DNA could be amplified, then proceed sequencing, and coded as SEQ ID No.3 (part of 26S rDNA sequence).

Input the above sequences into NCBI database for sequence comparison, and the results show that it is 98% or higher identity with the 26S rDNA sequence of Yarrowia lipolytica E122, and 98% or higher identity with the 26S rDNA sequence of Yarrowia lipolytica W29 (CLIB89). Therefore, it can be determined that the yeast that can synthesize erythritol screened in the present invention is Yarrowia lipolytica or Candida lipolytica.

The inventor induce mutagenesis to the yeast with compound chemical reagents and in combination with adaptive evolution, raise the fermentation temperature from 30° C. to 35° C. The methods adopted are as follows:

Suspend the fresh yeast with 1.5% ethyl methyl sulfonate (EMS) and 0.5% diethyl sulfate (DES) for 1-10 hours, spread separately in hypertonic YPD culture (300 g/L of anhydrous glucose, 10 g/L of yeast cell powder, 5 g/L of ammonium citrate, 3 g/L of peptone, 15 g/L of agar, PH5.5), and culture at 35° C. for 10 days. Proceed pure culture to the newly grown colonies, and adaptive evolution at 35° C. After 180 days of high-temperature adaptive evolution, select a single colony with vigorous growth for testing of erythritol synthesis by fermentation at 35° C., composition of fermentation medium: 300 g/L of anhydrous glucose, 8 g/L of yeast cell powder, 5 g/L of ammonium citrate, 3 g/L of peptone, 0.02 g/L of copper chloride, 0.02 g/L of manganese chloride, 0.05 g/L of vitamin B1, initial pH5.5. Through fermentation test, it is found that one strain still keep the same efficiency of synthesizing erythritol with its wild-type at 35° C., and most of the other strains could grow at 35° C., but synthesize more mannitol. Name the new strain which can grow well at 35° C. and synthesize erythritol efficiently as ery929. The yield of erythritol synthesized from 300 g/L glucose have reached 174 g/L. The strain ery929 is now preserved at China General Microbiological Culture Collection Center (CGMCC), with the deposit number of CGMCC No. 18478.

### Embodiment 2 Construct the Recombinant Yarrowia lipolytica Strain Capable of Synthesizing Xylitol

(1) Overexpress the Xylitol Dehydrogenase Gene in Yarrowia lipolytica.

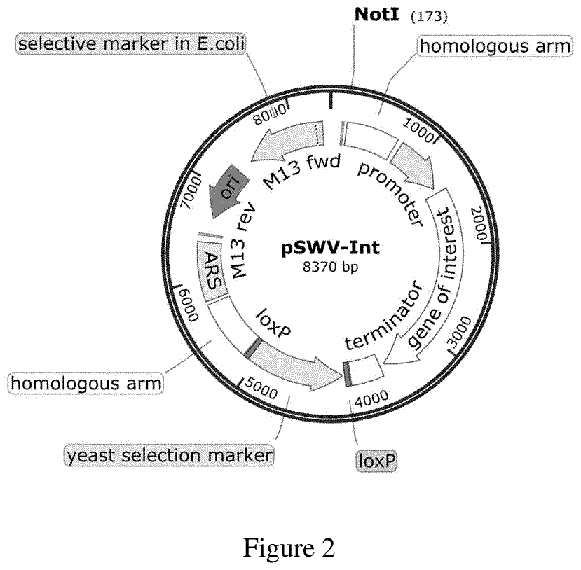

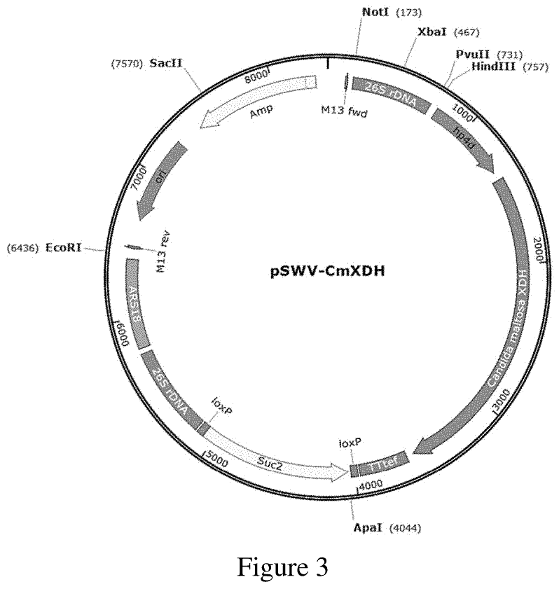

Clone the xylitol dehydrogenase gene of the optimally synthsized Candida maltosa (SEQ ID NO. 9) to the integrative expression plasmid vector pSWV-Int (FIG. 2). The vector is based on the common cloning vector pUC series, adding common DNA element sequence, such as 26S rDNA left and right homologous arm sequence, synthetic promoter hp4d sequence, terminator TT_{TEF} sequence of transcriptional extension factor gene, sucrase selective marker gene sequence Suc2, Escherichia coli plasmid replication origin sequence, DNA element of ampicillin resistance gene sequence, these are basic DNA elements and those skilled in the art can check it up from NCBI database (https://www.ncbi.nlm.nih.gov/). The constructed integrated expression vector containing xylitol dehydrogenase gene is shown in FIG. 3, in which the xylitol dehydrogenase gene can also be replaced by other xylitol dehydrogenase genes (e.g., xylitol dehydrogenase gene of Gluconobacter oxydans, etc.), and the selective marker Suc2 can be replaced by hytromycin resistance genes, while other DNA elements remain unchanged. Use NotI and EcoRI to linearize the vector, and convert the Yarrowia lipolytica ery929 that synthesize erythritol or other Yarrowia lipolytica that do not synthesize erythritol such as CLIB122, then screen on minimal medium containing sucrose. Composition of the medium used for screening: 6 g/L of yeast nitrogen base, 5 g/L of ammonium sulfate, 10 g/L of saccharose, 15 g/L of agar powder, pH 6.0. Since Yarrowia lipolytica ery929 strain cannot utilize sucrose, transformants that can grow in minimal medium containing sucrose do contain sucrase, which hydrolyzes sucrose into glucose and fructose, and also contain xylitol dehydrogenase gene, which can reduce xylulose to xylitol.

Then, transform the plasmid pUB4-CRE containing Cre recombinase into mutants expressing xylitol dehydrogenase, and screen in YPD agar medium containing hygromycin as selective marker (10 g/L of glucose, 10 g/L of yeast cell powder, 5 g/L of peptone, 15 g/L of agar, 300 μg/ml of hygromycin, pH 6.0). Transfer the resulting transformants to the minimal medium containing sucrose (6 g/L of yeast nitrogen base, 5 g/L of ammonium sulfate, 10 g/L of sucrose, 15 g/L of agar powder, pH 6.0), and select mutants with sucrase gene loss (i.e., no reuse of sucrose). Then, culture the mutants that can not use sucrose in hygromycin free liquid YPD, and then apply gradient dilution and spread on the hygromycin free solid YPD. Select the mutants that can not resist hygromycin from the resulting transformants and transfer to YPD containing hygromycin, that is, the overexpression of xylitol dehydrogenase gene. In the meanwhile, screen mutants with loss of marker sucrase gene (Suc2), which can be used in hosts that overexpress other genes. Proceed total RNA isolation to the mutant and perform reverse transcription, and use reverse transcription products as templates for fluorescence quantitative PCR (RT-qPCR) to detect the expression level of xylitol dehydrogenase gene. Compared with the control strain ery929, it is found that the xylitol dehydrogenase gene of the mutant strain has obvious amplification curve, while the control strain has no amplification curve, indicating that xylitol dehydrogenase gene get expressed in the mutant strain.

Inoculate the mutants that overexpressed xylitol dehydrogenase gene and lost sucrase gene (Suc2) in fermentation medium for xylitol synthesis test. Composition of fermentation medium: 200 g/L of glucose, 8 g/L yeast cell powder, 5 g/L of peptone, 3 g/L of ammonium citrate, 0.05 g/L of zinc chloride, 0.01 g/L of manganese chloride, 0.05 g/L of vitamin B1, pH6.0. Take samples periodically for detection, 85 hours to run out of glucose, and the content of xylitol, erythritol and mannitol are 0.2 g/L, 96.4 g/L and 12 g/L, respectively. If the xylitol dehydrogenase gene of the above mentioned Candida maltosa are replaced by the xylitol dehydrogenase gene of Gluconobacter oxydans, and transformed into strain ery929, the fermentation test results show that the content of xylitol, erythritol and mannitol are 0.3 g/L, 90.2 g/L and 11 g/L, respectively. If the xylitol dehydrogenase gene of the above mentioned Candida maltosa are substituted by the xylitol dehydrogenase gene of Debaryomyces hansenii, and transformed into strain ery929, the fermentation test results show that the content of xylitol, erythritol and mannitol are 0.2 g/L, 98.6 g/L and 13 g/L, respectively. From the results of fermentation, it can be seen that the yield of synthesis of xylitol by Yarrowia lipolytica is very low if only xylitol dehydrogenase gene is overexpressed.

When the above expression vector is used to transform the Yarrowia lipolytica CLIB122, which can not produce erythritol, under the same conditions for 90 hours, it is found that neither xylitol nor erythritol can be detected, but 6 g/L of mannitol and a large amount of glucose (153 g/L) is detected.

(2) Overexpress the 5-p Xylitol Dehydrogenase Gene (also known as 5-p Xylulose Reductase) in Yarrowia lipolytica.

Respectively replace the xylitol dehydrogenase gene of the integrated expression vector pSWV-CmXDH in step (1) with the 5-p xylulose reductase genes of Clostridioides difficile, Lactobacillus rhamnosus, and Lactobacillus plantarum (SEQ ID NO. 14, SEQ ID NO. 15, SEQ ID NO. 17, SEQ ID NO. 20 et al. in the sequence table) to obtain the integrated expression vector containing 5-p xylulose reductase gene. Transform the Yarrowia lipolytica ery929 to obtain the transformant containing 5-p xylulose reductase gene. Fermentation under the same conditions as in step (1), the test results show that the contents of xylitol, erythritol and mannitol are 0.3-0.7 g/L, 92-98 g/L and 10-12 g/L, respectively. The results show that the yield of xylitol from glucose by Yarrowia lipolytica is still very low if only 5-p xylulose reductase gene is contained. In order to testify that the gene is expressed in the cell, perform total RNA isolation to the transformant and reverse transcription, and use reverse transcription products as templates for fluorescence quantitative PCR to detect the expression level of 5-p xylulose reductase gene. Compared with the control strain ery929, it is found that the 5-p xylulose reductase gene of the mutant strain has obvious amplification curve, while the control strain has no amplification curve, indicating that 5-p xylulose reductase gene get expressed in the transformant.

(3) Overexpress the 5-p Xylulose Phosphatase Gene in Yarrowia lipolytica.

Respectively replace the xylitol dehydrogenase gene of the integrated expression vector in step (1) with the genes that containing the activity 5-p xylulose reductase from Kluyveromyces marxianus, Saccharomyces cerevisiae, Komagataella phaffii, Lactobacillus plantarum and Bacillus subtilis (SEQ ID NO. 21, SEQ ID NO. 22, SEQ ID NO. 24 et al. in the sequence table) to obtain the integrated expression vector containing 5-p xylulose phosphatase gene. Transform the Yarrowia lipolytica ery929 to obtain the transformant contaning 5-p xylulose phosphatase gene. Fermentation under the same conditions as in step (1), the test results show that no xylitol is detected by liquid chromatography, and the contents of erythritol and mannitol are 95-102 g/L and 10-12 g/L, respectively. The results show that Yarrowia lipolytica can not synthesize xylitol from glucose if only 5-p xylulose phosphatase gene is contained. In order to testify that the 5-p xylulose phosphatase gene is expressed in the transformant, the inventor performed quantitative PCR analysis. The specific operations are as follows: perform total RNA isolation to the transformant (use the Trizol for extraction), then reverse transcription (use the commercial reverse transcription kit), and take 2 microliter reverse transcription products for fluorescence quantitative PCR (use the 2 microliter reverse transcription products), operate in fluorescence quantitative PCR instrument as a 20 microliter reaction system. After the reaction, it is found that the transformant has amplification curve and the gene is amplified, while the control strain has no amplification, indicating that the gene get expressed in the transformant.

(4) Overexpress the Xylitol Transporter Gene or NADP Transhydrogenase Gene in Yarrowia lipolytica.

Respectively replace the xylitol dehydrogenase gene of the integrated expression vector in step (1) with the xylitol transporter gene or NADP transhydrogenase gene, to obtain the integrated expression vector containing xylitol transporter gene or NADP transhydrogenase gene. Transform the Yarrowia lipolytica ery929 to obtain the transformant contaning xylitol transporter gene or NADP transhydrogenase gene. Fermentation under the same conditions as in step (1), the test results show that no xylitol is detected, and the contents of erythritol and mannitol are 96-104 g/L and 9-12 g/L, respectively. The results show that Yarrowia lipolytica can not synthesize xylitol from glucose if only xylitol transporter gene or NADP transhydrogenase gene is contained. Fluorescence quantitative PCR detection shows that the transformant has amplification curve and the gene is amplified, while the control strain has no amplification, indicating that the NADP transhydrogenase gene get expressed in the transformant.

The above-mentioned results indicate that recombinant Yarrowia lipolytica can only produce a small amount of xylitol if it contains only xylitol dehydrogenase or 5-p xylulose reductase gene, however, xylitol synthesis can not be detected if the recombinant strain contains only 5-p xylulose phosphatase gene, xylitol transporter or NADP transhydrogenase gene. In order to verify the synergistic effect of these five genes, transfer the five genes into Yarrowia lipolytica to test whether the synthesis efficiency of xylitol is improved.

(5) Acquisition of Yarrowia lipolytica ery959 that can Simultaneously Express Five Genes: Xylitol Dehydrogenase Gene, 5-p Xylulose Reductase Gene, 5-p Xylulose Phosphatase Gene, Xylitol Transporter Gene and NADP Transhydrogenase Gene.

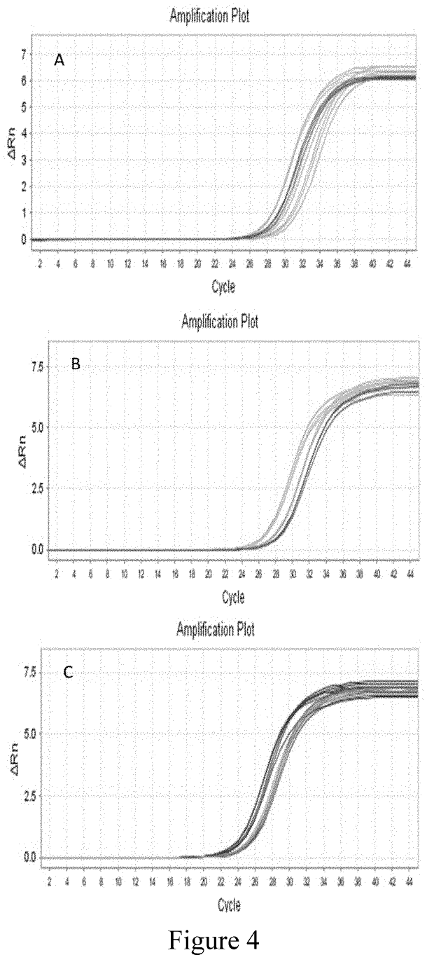

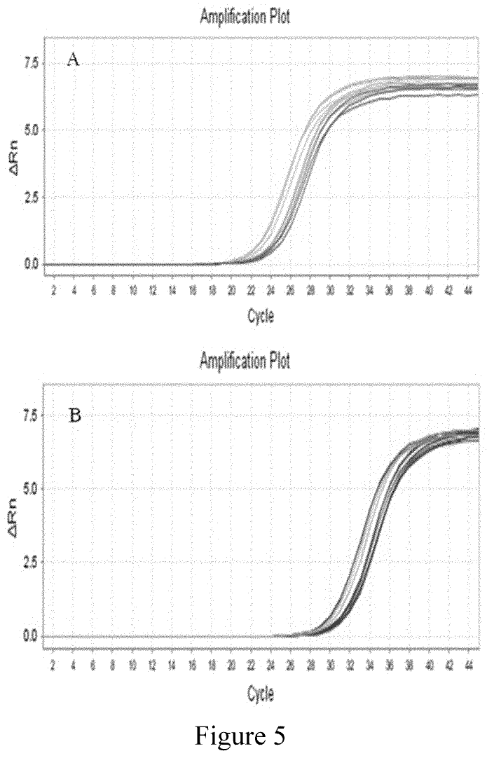

Using the recombinant yeast that overexpressed xylitol dehydrogenase gene of Gluconobacter oxidans in step (1) and recovered the sucrase maker (Suc2) as the host, transfer respectively the 5-p xylitol dehydrogenase gene (SEQ ID NO. 14), 5-p xylulose phosphatase gene (SEQ ID NO. 31), xylitol transporter gene (SEQ ID NO. 32) and NADP transhydrogenase gene (SEQ ID NO. 44) into Yarrowia lipolytica for expression. Refer to step (1) for the methods of transformation and recovery of selective markers. Obtain the recombinant Yarrowia lipolytica ery959 that can express the above five genes simultaneously. In order to verify that the five genes in ery959 are expressed, the inventor processed the total RNA isolaton, reverse transcription and fluorescence quantitative detection, and found that the five genes had typical amplification curves, indicating that the five introduced exogenous genes got expressed. The amplification curves are shown in FIGS. 4 and 5 (A-C in FIG. 4 are the amplification curves of xylitol dehydrogenase gene, 5-p xylulose reductase gene and 5-p xylulose phosphatase gene respectively; A-B in FIG. 5 are the amplification curves of xylitol transporter gene and NADP hydrogenase gene respectively). The method of xylitol synthesis by fermentation of the recombinant yeast is the same as step (1). After 98 hours of fermentation, glucose are exhausted, and result in that the content of xylitol, erythritol and mannitol are 3.6 g/L, 82.5 g/L and 7.2 g/L respectively, pH3.2 at the end of fermentation.

According to the above results, xylitol production cannot be greatly improved by expressing genes related to xylitol synthesis in Yarrowia lipolytica, and erythritol is still synthesized in large quantities. The reason may be that 5-p xylulose, the precursor of xylitol synthesis, still flows into the pathway of erythritol synthesis mainly through ketotransferase. Therefore, it is possible to significantly improve the synthesis of xylitol by further knocking out the transketolase gene and blocking the pathway of 5-p xylulose into the synthesis of erythritol.

(6) Knocking out the Transketolase Gene on the Basis of ery959 to Obtain the Mutant ery959 ΔTKL12.

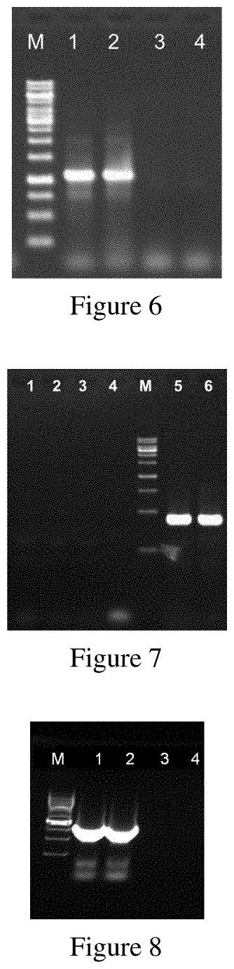

Construct and synthesize respectively the gene disruption cassettes of transketolase gene 1 (YlTKL1) and transketolase gene 2 (YlTKL2), and transform the Yarrowia lipolytica strain obtained in step (5), then knock out the two transketolase genes. Gene disruption cassettes successively contains 1 kb-1.5 kb bases upstream of the transketolase gene, retrievable selective markers (sucrase gene, with 1oxP sites at both ends of the gene, facilitating the recovery of selective markers), and 1 kb-1.5 kb bases downstream of the transketolase gene. After synthesis, the transketolase gene disruption cassettes are used to transform the Yarrowia lipolytica obtained in step (5), and screen in minimal medium supplemented with sucrose and ammonium sulfate (6 g/L of yeast nitrogen base, 5 g/L of ammonium sulfate, 10 g/L of sucrose, 0.05 g/L each of phenylalanine, tyrosine and tryptophan, 15 g/L of agar powder, pH 6.0). Since the Yarrowia lipolytica strain obtained in step (5) cannot utilize sucrose, transformants that can grow in minimal medium containing sucrose do contain sucrose gene (Suc2), which hydrolyzes sucrose into glucose and fructose, and thus can grow. Extract the genome of the mutant and amplify by PCR with primers of P_{TKL1-F}/P_{TKL1-R} and P_{TKL2-F}/P_{TKL2-R} (primer sequences: SEQ ID NO. 46-49). The results show that both transketolase gene fragments of the control strain can be amplified (about 1100 bp DNA fragment), while the mutant can not, indicating that the two transketolase gene are knocked out (FIG. 6, in which, YlTKL1 gene of the control strain ery929 can be amplified; The YlTKL2 gene of control strain ery929 can be amplified. The YlTKL1 gene can not be amplified after being knocked out from the mutant. The YlTKL2 gene can not be amplified after being knocked out from the mutant).

Primer sequences used to amplify YlTKL1 gene fragment:

P_{TKL1-F}:
(SEQ ID NO. 46)
5′-tgaataggagacttgacagtctggc-3′
P_{TKL1-R}:
(SEQ ID NO. 47)
5′-ctctgagatcatccgagcattcaag-3

Primer sequences used to amplify YlTKL2 gene fragment:

P_{TKL2-F}:
(SEQ ID NO. 48)
5′-atgccccctttcaccctggcagacac-3′
P_{TKL2-R}:
(SEQ ID NO. 49)
5′-ctataacccggcacagagccttggcg-3′

Then, transform the plasmid pUB4-CRE containing Cre recombinase into mutant with both YlArDH1 and YlArDH2 knocked out, and screen in YPD agar medium containing hygromycin as selective marker (10 g/L of glucose, 10 g/L of yeast cell powder, 5 g/L of peptone, 0.05 g/L each of phenylalanine, tyrosine and tryptophan, 15 g/L of agar, 300 μg/ml of hygromycin, pH 6.0). Transfer the resulting transformants to the minimal medium containing sucrose (6 g/L of yeast nitrogen base, 5 g/L of ammonium sulfate, 10 g/L of sucrose, 0.05 g/L each of phenylalanine, tyrosine and tryptophan, 15 g/L of agar powder, pH 6.0), and select mutants with sucrase gene loss (i.e., no reuse of sucrose). Then, culture the mutants that can not use sucrose in hygromycin free liquid YPD, and then apply gradient dilution and spread on the hygromycin free solid YPD. Select the mutants that can not resist hygromycin from the resulting transformants and transfer to YPD containing hygromycin, that is, the mutant with the transketolase gene being knocked out and the sucrase gene has lost. The mutant can simultaneously express xylitol dehydrogenase gene, 5-p xylulose reductase gene, 5-p xylulose phosphatase gene, xylitol transporter gene and NADP transhydrogenase gene, in the meanwhile, the transketolase gene is knocked out. It can be used as host for other gene knockout. The sequence codes of gene disruption cassettes of transketolase genes 1 and 2 are SEQ ID NO. 50 and SEQ ID NO. 51, respectively.

Conduct the test of xylitol synthesis from glucose by fermentation with mutant ery959ΔTKL12 in the fermentation medium same to the fermentation medium in step (1), and supplemented with 0.05 g/L each of phenylalanine, tyrosine and tryptophan. Periodically take samples to detect glucose and product production, and it is found that the glucose utilization rate decreased significantly, the control strain ery959 can run out of glucose within 90 hours, and the cell OD₆₀₀ is 22.5, while the mutant strain ery959ΔTKL12 still can not use up glucose at 220 hours (sterile water is added during the period to compensate for volatile water). The contents of xylitol, mannitol, arabitol, ribitol and residual glucose are 23 g/L, 36 g/L, 3 g/L, 3 g/L and 84 g/L, respectively. The OD₆₀₀ is 22.5, and no erythritol are detected, indicating that the knocking out of transketolase gene plays a very important role in the synthesis of xylitol and erythritol. It also indicates that knocking out of TKL gene can inhibit cell growth, and the addition of three aromatic amino acids (phenylalanine, tyrosine, and tryptophan) could not completely restore the density of control strain ery929. The known literature also demonstrates that, transketolase is a key enzyme in the synthesis of erythritol, and its activity is very high (Sawada et al. 2009. Key roles in transketolase activity in erythritol production by Trichosporonoides megachiliensis SN-G42. Journal of Bioscience and Bioengineering, 108: 385-390) Since cell growth is inhibited after the transketolase gene is knocked out and glucose use is significantly slower, therefore, in order to increase the cell growth and glucose utilization rate appropriately, transfer in the transketolase gene YlTKL1 with weakened promoter on the basis of strain ery959ΔTKL12 whose transketolase gene is knocked out, to partially restore the expression of transketolase gene 1. Conduct gene fusion between the weak promoter sequence (SEQ ID NO. 78) and the 5′ end of the transketolase YlTKL1 gene (SEQ ID NO. 74), to form a new sequence coded as SEQ ID NO. 79, and transform to ery959ΔTKL12, then screen on minimal medium (composition: 6 g/L of yeast nitrogen base, 10 g/L of glucose, 5 g/L of ammonium sulfate, 15 g/L of agar powder, pH6.5, without phenylalanine, tyrosine and tryptophan). Since the ery959ΔTKL12 cannot grow on the minimal medium without phenylalanine, tyrosine, and tryptophan, hence the newly grown transformant contains SEQ ID NO. 79 (down-regulate the transketolase gene), which is named ery959ΔTKL.

Conduct the test of xylitol synthesis from glucose by fermentation with mutant ery959 ΔTKL in the fermentation medium same to the fermentation medium in step (1) without phenylalanine, tyrosine and tryptophan. Periodically take samples to detect the composition of fermentation broth, and it is found that the glucose utilization rate becomes significantly faster, and the chromatographic analysis shows that the contents of xylitol, mannitol, arabitol, ribitol and erythritol are 58 g/L, 23 g/L, 3 g/L, 3 g/L and 5 g/L, respectively, and the cell OD₆₀₀ is 18.4.

Although knocking out or down-regulating the expression of transketolase gene can result in a significant decrease in the content of erythritol, more mannitol and arabitol are synthesized. Therefore, further knockout of mannitol dehydrogenase and arabinol dehydrogenase genes can theoretically reduce or block the synthesis of mannitol and arabinol.

(7) Knock out the Mannitol Dehydrogenase Gene of Mutant ery959ΔTKL to Obtain the Strain ery959ΔTKLΔMDH with the mannitol dehydrogenase gene knocked out.

Construct and synthesize respectively the gene disruption cassettes of mannitol dehydrogenase gene 1 (YlMDH1) and mannitol dehydrogenase gene 2 (YlMDH2), and transform the Yarrowia lipolytica strain ery959ΔTKL, then knock out the two mannitol dehydrogenase genes. Gene disruption cassettes successively contains 1 kb-1.5 kb bases upstream of the gene, retrievable selective markers (such as aminocyclitol phoshotransferase gene, sucrase gene, with 1oxP sites at both ends of the gene, facilitating the recovery of selective markers), and 1 kb-1.5 kb bases downstream of the gene. After synthesis, it is used to transform Yarrowia lipolytica ery959ΔTKL, then screen in the minimal medium supplemented with sucrose and ammonium sulfate (6 g/L of yeast nitrogen base, 5 g/L of ammonium sulfate, 10 g/L of sucrose, 15 g/L of agar powder, pH 6.0). Since the Yarrowia lipolytica ery959ΔTKL cannot utilize sucrose, transformants that can grow in minimal medium containing sucrose do contain sucrase, which hydrolyzes sucrose into glucose and fructose, and thus can grow. Since the sucrase gene is located in the middle of the upstream and downstream homologous sequence of the mannitol dehydrogenase gene in the gene disruption cassette, there are mutants with mannitol dehydrogenase gene knocked out in the transformants, and the mannitol dehydrogenase gene is replaced by sucrase gene in the mutant. Extract the genome of the mutant and perform PCR with the primers of the two mannitol dehydrogenase genes (sequences of peimers are SEQ ID NO. 52, SEQ ID NO. 53, SEQ ID NO. 54, SEQ ID NO. 55). The mannitol dehydrogenase gene of the control strain can be amplified (about 900 bp target DNA fragment), while that of the mutant can not, indicating that the mannitol dehydrogenase gene is indeed knocked out (FIG. 7, where, lane 1: the YlMDH1 gene fragment can not be amplified after the YlMDH1 gene is knocked out from mutant 1; lane 2: the YlMDH2 gene fragment can not be amplified after the YlMDH2 gene is knocked out from mutant 1; lane 3: the YlMDH1 gene fragment can not be amplified after the YlMDH1 gene is knocked out from mutant 2; lane 4: the YlMDH2 gene fragment can not be amplified after the YlMDH2 gene is knocked out from mutant 2; M: DNA molecular weight standards; lane 5: the YlMDH1 gene fragment of control strain ery929 can be amplified (900 bp); lane 6: the YlMDH2 gene fragment of control strain ery929 can be amplified (900 bp)).

Primer sequences used to amplify YlMDH1 gene fragment:

P_{MDH1-F}:
(SEQ ID NO. 52)
5′-ctatctccacaacaatgcctgcaccag-3′
P_{MDH1-R}:
(SEQ ID NO. 53)
5′-ccggttacacatgactgtaggaaac-3

Primer sequences used to amplify YlMDH2 gene fragment:

P_{MDH2-F}:
(SEQ ID NO. 54)
5′-ccatacacagcaccacctcaatc-3′
P_{MDH2-R}:
(SEQ ID NO. 55)
5′-tctatatacatcctctaaggagc-3′

Then, transform the plasmid containing Cre recombinase (pUB4-CRE, from the following references: Fickers et al. 2003. New disruption cassettes for rapid gene disruption and marker rescue in the yeast Yarrowia Methods, 55, 727-737) into mutants that YlMDH1 and YlMDH2 have been knockout, and recover sucrase selective markers. Screen in YPD agar medium containing hygromycin as selective marker (10 g/L of glucose, 10 g/L of yeast cell powder, 5 g/L of peptone, 15 g/L of agar, 300 μg/ml of hygromycin, pH 6.0). Transfer the resulting transformants to the minimal medium containing sucrose (6 g/L of yeast nitrogen base, 5 g/L of ammonium sulfate, 10 g/L of saccharose, 15 g/L of agar powder, pH 6.0), and select mutants with sucrase gene loss (i.e., no reuse of sucrose). Then, culture the mutants that can not use sucrose in hygromycin free liquid YPD, and then apply gradient dilution and spread on the hygromycin free solid YPD. Select the mutants that can not resist hygromycin from the resulting transformants and transfer to YPD containing hygromycin, that is, the knocking out of mannitol dehydrogenase gene. In the meanwhile, mutants with loss of sucrase gene, can be used as host for other gene knockout. The sequence codes of gene disruption cassettes of mannitol dehydrogenase genes 1 and 2 are shown in SEQ ID NO. 56 and SEQ ID NO. 57, respectively.

Conduct the test of xylitol synthesis from glucose by fermentation with mutant ery959 ΔTKLΔMDH in the fermentation medium same to the fermentation medium in step (1). Take samples periodically for detection, 104 hours to run out of glucose, and the content of xylitol, erythritol and ribitol are 86 g/L, 5 g/L and 3 g/L, respectively, as well as no mannitol and arabitol are detected. It can be seen that knockout of the mannitol dehydrogenase gene can eliminate both mannitol and arabitol, the by products, but ribitol is still produced. In order to eliminate ribitol, the inventor carried out an experiment to knock out the arabinol dehydrogenase gene.

(8) Knock out the Arabinitol Dehydrogenase Gene of Mutant ery959ΔTKLΔMDH to Obtain the Strain ery959ΔTKLΔMDHΔArDH with the Arabinitol Dehydrogenase Gene Knocked out.

Construct and synthesize respectively the gene disruption cassettes of arabinitol dehydrogenase gene 1 (YlArDH1) and arabinitol dehydrogenase gene 2 (YlArDH2), and transform the Yarrowia lipolytica strain ery959ΔTKLΔMDH, then knock out the two arabinitol dehydrogenase genes. Gene disruption cassettes successively contains 1 kb-1.5 kb bases upstream of the gene, retrievable selective markers (sucrase gene, with 1oxP sites at both ends of the gene, facilitating the recovery of selective markers), and 1 kb-1.5 kb bases downstream of the gene. After synthesis, the arabinitol gene disruption cassettes are used to transform the Yarrowia lipolytica ery959ΔTKLΔMDH with the mannitol dehydrogenase gene knocked out, and screen in minimal medium supplemented with sucrose and ammonium sulfate (6 g/L of yeast nitrogen base, 5 g/L of ammonium sulfate, 10 g/L of sucrose, 15 g/L of agar powder, pH 6.0). Since the Yarrowia lipolytica strain with the arabinitol dehydrogenase gene knocked out cannot utilize sucrose, transformants that can grow in minimal medium containing sucrose do contain sucrase, which hydrolyzes sucrose into glucose and fructose, and thus can grow. Extract the genome of the transformant in the mutant and amplify by PCR with primers of P_{ArDH1-F}/P_{ArDH1-R} and P_{ARDH2-F}/P_{ArDH2-R} (primer sequences: SEQ ID NO. 58, SEQ ID NO. 59, SEQ ID NO. 60, SEQ ID NO. 61). The results show that the arabinitol dehydrogenase gene of the control strain can be amplified (about 900 bp DNA fragment), while the mutant can not, indicating that the two arabinitol dehydrogenase genes are knocked out (FIG. 8, where, lane 1: the YlArDH1 gene of the control strain ery929 can be amplified; lane 2: the YlArDH2 gene of control strain ery929 can be amplified; lane 3: the YlArDH1 gene can not be amplified after being knocked out from the mutant; lane 4: the YlArDH2 gene can not be amplified after being knocked out from the mutant).

Primer sequences used to amplify YlArDH1 gene fragment:

P_{ArDH1-F}:
(SEQ ID NO. 58)
5′- accagatggtgtaacctccatcgac-3′
P_{ArDH1-R}:
(SEQ ID NO. 59)
5′-ggaagtggtggtctgggtatcgcag-3

Primer sequences used to amplify YlArDH2 gene fragment:

P_{ArDH2-F}:
(SEQ ID NO. 60)
5′-cacatacaccacaacacacacaaaatc-3′
P_{ArDH2-R}:
(SEQ ID NO. 61)
5′-ttcctctgagacaatcgcgtcggatc-3′

Then, transform the plasmid pUB4-CRE containing Cre recombinase into mutant with both YlArDH1 and YlArDH2 knocked out, to recover sucrase selective markers. Screen in YPD agar medium containing hygromycin as selective marker (10 g/L of glucose, 10 g/L of yeast cell powder, 5 g/L of peptone, 15 g/L of agar, 300 μg/ml of hygromycin, pH 6.0). Transfer the resulting transformants to the minimal medium containing sucrose (6 g/L of yeast nitrogen base, 5 g/L of ammonium sulfate, 10 g/L of sucrose, 15 g/L of agar powder, pH 6.0), and select mutants with sucrase gene loss (i.e., no reuse of sucrose). Then, culture the mutants that can not use sucrose in hygromycin free liquid YPD, and then apply gradient dilution and spread on the hygromycin free solid YPD. Select the mutant ery959ΔTKLΔMDHΔArDH that can not resist hygromycin from the resulting transformants and transfer to YPD containing hygromycin, that is, the knocking out of arabinitol dehydrogenase gene. In the meanwhile, mutants with loss of sucrase gene, can be used as host for other gene knockout. The sequence codes of gene disruption cassettes of arabinitol dehydrogenase genes 1 and 2 are SEQ ID NO. 62 and SEQ ID NO. 63.

Conduct the test of xylitol synthesis from glucose by fermentation with mutant ery959ΔTKLΔMDHΔArDH in the fermentation medium same to the fermentation medium in step (1). Take samples periodically for detection, 106 hours to run out of glucose, and the content of xylitol and erythritol are 87 g/L and 6 g/L respectively, and no mannitol, arabitol and ribitol are detected.

(9) Knock out the 5-p Ribulose Isomerase Gene of the Mutant ery959ΔTKLΔMDHΔArDH to Obtain the Yarrowia lipolytica ery959ΔTKLΔMDHΔArDHΔRPI with the 5-p Ribulose Isomerase Gene Knocked out

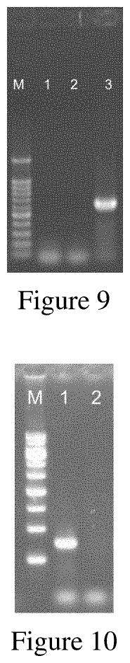

Construct and synthesize the gene disruption cassette of 5-p ribulose isomerase gene (RPI), and transform the Yarrowia lipolytica strain ery959ΔTKLΔMDHΔArDH, then knock out the RPI. Gene disruption cassettes successively contains 1 kb-1.5 kb bases upstream of the 5-p ribulose isomerase gene, retrievable selective markers (sucrase gene, with 1oxP sites at both ends of the gene, facilitating the recovery of selective markers), and 1 kb-1.5 kb bases downstream of the 5-p ribulose isomerase gene. After synthesis, the 5-p ribulose isomerase gene disruption cassette is used to transform the Yarrowia lipolytica ery959ΔTKLΔMDHΔArDH, and screen in minimal medium supplemented with sucrose and ammonium sulfate (6 g/L of yeast nitrogen base, 5 g/L of ammonium sulfate, 10 g/L of sucrose, 15 g/L of agar powder, pH 6.0). Since the above-mentioned Yarrowia lipolytica strain with the transketolase gene, mannitol dehydrogenase gen, arabinitol dehydrogenase gene knocked out cannot utilize sucrose, hence transformants that can grow in minimal medium containing sucrose do contain sucrase, which hydrolyzes sucrose into glucose and fructose, and thus can grow. Extract the genome of the mutant and amplify by PCR with primer of P_{RPI-F}/P_{RPI-R} (primer sequence: SEQ ID NO. 64-65). The results show that the 5-p ribulose isomerase gene fragments of the control strain can be amplified (about 600 bp DNA fragment), while the mutant can not, indicating that the 5-p ribulose isomerase gene is knocked out (FIG. 9, where, lane 1-2: the RPI gene cannot be amplified after RPI gene is knocked out from the mutant; lane 3: the RPI gene of control strain can be amplified).

Primer sequences used to amplify YlRPI gene fragment:

P_{RPI-F}:
(SEQ ID NO. 64)
5′-aactgcctcctcttgagcaggccaag-3′
P_{RPI-R}:
(SEQ ID NO. 65)
5′-ggaacagcagcttgatcttgatgtgc-3

Transform the plasmid pUB4-CRE containing Cre recombinase into the mutant with RPI gene knocked out. Refer to the method described above or retrieving sucrase selective markers. Sequence of 5-p ribulose isomerase gene disruption cassette is SEQ ID NO. 66.

Conduct the test of xylitol synthesis from glucose by fermentation with mutant ery959 ΔTKLΔMDHΔArDHΔRPI in the fermentation medium same to the fermentation medium in step (1). Take samples periodically for detection, 102 hours to run out of glucose, and the content of xylitol and erythritol are 92.3 g/L and 6.4 g/L respectively, and no mannitol, arabitol and ribitol are detected.

(10) Knock out the Xylulose Kinase Gene of the Mutant ery959ΔTKLΔMDHΔArDHΔRPI to obtain the Yarrowia lipolytica ery959 ΔTKLΔMDHΔArDHΔRPIΔXKS1 with the Xylulose Kinase Gene Knocked out.

Construct and synthesize the gene disruption cassette of xylulose kinase gene (Y/XKS1), and transform the Yarrowia lipolytica strain ery959ΔTKLΔMDHΔArDHΔRPI, then knock out the YlXKS1. Gene disruption cassette successively contains 1 kb-1.5 kb bases upstream of the xylulose kinase gene, retrievable selective markers (sucrase gene, with 1oxP sites at both ends of the gene, facilitating the recovery of selective markers), and 1 kb-1.5 kb bases downstream of the xylulose kinase gene. After synthesis, the xylulose kinase gene disruption cassette is used to transform the Yarrowia lipolytica ery959ΔTKLΔMDHΔArDHΔRPI, and screen in minimal medium supplemented with sucrose and ammonium sulfate (6 g/L of yeast nitrogen base, 5 g/L of ammonium sulfate, 10 g/L of sucrose, 15 g/L of agar powder, pH 6.0). Since the Yarrowia lipolytica strain with the transketolase gene knocked out cannot utilize sucrose, transformants that can grow in minimal medium containing sucrose do contain sucrase, which hydrolyzes sucrose into glucose and fructose, and thus can grow. Extract the genome of the mutant and amplify by PCR with primer of P_{XKS1-F}/P_{XKS1-R} (primer sequence: SEQ ID NO. 67-68). The results show that the xylulose kinase gene fragments of the control strain can be amplified (about 800 bp DNA fragment), while the mutant can not, indicating that the xylulose kinase gene is knocked out (FIG. 10, where, lane 1: the YlXKS1 gene of control strain ery929 can be amplified; lane 2: the YlXKS1 gene cannot be amplified after YlXKS1 gene is knocked out from the mutant).

Primer sequences used to amplify YlXKS1 gene fragment (the amplified product is 0.8 kb):

P_{XKS1-F}:
(SEQ ID NO. 67)
5′-gactggatctttcgactcaacagctc-3′
P_{XKS1-R}:
(SEQ ID NO. 68)
5′-ccaaagacacaatcacgtcattggcc-3

Then, transform the plasmid pUB4-CRE containing Cre recombinase into the mutant with YlXKS1 gene knocked out, and screen in YPD agar medium containing hygromycin as selective marker (10 g/L of glucose, 10 g/L of yeast cell powder, 5 g/L of peptone, 15 g/L of agar, 300 μg/ml of hygromycin, pH 6.0). Transfer the resulting transformants to the minimal medium containing sucrose (6 g/L of yeast nitrogen base, 5 g/L of ammonium sulfate, 10 g/L of sucrose, 15 g/L of agar powder, pH 6.0), and select mutants with sucrase gene loss (i.e., no reuse of sucrose). Then, culture the mutants that can not utilize sucrose in hygromycin free liquid YPD, and then apply gradient dilution and spread on the hygromycin free solid YPD. Select the mutants that can not resist hygromycin from the resulting transformants and transfer to YPD containing hygromycin, that is, the mutant ery959ΔTKLΔMDHΔArDHΔRPIΔXKS1 with the xylulose kinase gene knocked out, and in the meanwhile, with loss of sucrase gene. Sequence of xylulose kinase gene disruption cassette is SEQ ID NO. 69.

Conduct the test of xylitol synthesis from glucose by fermentation with mutant ery959ΔTKLΔMDHΔArDHΔRPIΔXKS1 in the fermentation medium same to the fermentation medium in step (1). Take samples periodically for detection, 104 hours to run out of glucose, and the content of xylitol and erythritol are 98 g/L and 6.5 g/L, respectively.

As can be seen from the results of the above ten embodiments, the mutant ery959ΔTKLΔMDHΔArDHΔRPIΔXKS1 that overexpress five enzyme genes (xylitol dehydrogenase gene, 5-p xylulose reductase gene, 5-p xylulose phosphatase gene, xylitol transporter gene and NADP transhydrogenase gene), simultaneously with five enzyme genes (transketolase gene, mannitol dehydrogenase gene, arabinitol dehydrogenase gene, 5-p ribulose isomerase gene and xylulose kinase gene) knocked out and weak express transketolase gene 1 have the best effect on the synthesis of xylitol by fermentation. After 104 hours of fermentation from 200 g of anhydrous glucose, the fermentation broth contains 98 g of xylitol. Deposit this representative strain with the deposit number of CGMCC No.18479. The following steps are optimization tests that using the representative strain as an example to synthesize xylitol by fermentation.

(11) Synthesis of Xylitol using Yeast Strain CGMCC No.18479 at 25° C. and 50 g/L of Glucose.

Inoculate the recombinant yeast strains CGMCC No.18479 in a 2 L flask (using baffled flask to increase the effect of stirring dissolved oxygen) containing 500 ml of fermentation medium with an initial opitical density (OD₆₀₀) of 0.8. Fermentation medium components: 50 g/L of glucose, 2 g/L of yeast cell powder, 3 g/L of peptone, 1 g/L of hydrogen diamine phosphate, initial pH5.5, fermentation at 25° C. and 250 rpm. Take samples periodically to determine the content of glucose and xylitol. After 75 hours of fermentation, glucose is completely consumed, the content of xylitol is determined to be 12 g/L, and the conversion rate is 24%.

(12) Synthesis of Xylitol using Yeast Strain CGMCC No.18479 at 25° C. and 200 g/L of Glucose.

Inoculate the strains of recombinant yeast CGMCC No.18479 in a 2 L baffled flask containing 500 ml of fermentation medium with an initial opitical density (OD₆₀₀) of 0.8. Fermentation medium components: 200 g/L of glucose, 5 g/L of yeast cell powder, 5 g/L of peptone, 3 g/L of hydrogen diamine phosphate, 0.01 g/L of manganese chloride, 0.01 g/L of copper chloride, 0.01 g/L of zinc chloride, 0.02 g/L of magnesium sulfate, initial pH5.5, fermentation at 25° C. and 250 rpm. Take samples periodically to determine the content of glucose and xylitol. After 115 hours of fermentation, glucose is completely consumed, the content of xylitol is determined to be 96 g/L, and the conversion rate is 48%.

(13) Synthesis of Xylitol using Yeast Strain CGMCC No.18479 at 28° C. and 300 g/L of Glucose.

Inoculate the strains of recombinant yeast CGMCC No.18479 in a 2 L baffled flask containing 500 ml of fermentation medium with an initial opitical density (OD₆₀₀) of 0.8. Fermentation medium components: 300 g/L of glucose, 10 g/L of yeast cell powder, 5 g/L of peptone, 3 g/L of ammonium citrate, 0.02 g/L of manganese chloride, 0.01 g/L of copper chloride, 0.01 g/L of zinc chloride, 0.02 g/L of magnesium sulfate, initial pH5.5, fermentation at 28° C. at 250 rpm. Take samples periodically to determine the content of glucose and xylitol. After 140 hours of fermentation, glucose is completely consumed, the content of xylitol is determined to be 145 g/L, and the conversion rate is 48.3%.

(14) Synthesis of Xylitol using Yeast Strain CGMCC No.18479 at 30° C. and 300 g/L of Glucose.

Inoculate the strains of recombinant yeast CGMCC No.18479 in a 2 L baffled flask containing 500 ml of fermentation medium with an initial opitical density (OD₆₀₀) of 0.8. Fermentation medium components: 300 g/L of glucose, 10 g/L of yeast cell powder, 5 g/L of peptone, 3 g/L of ammonium citrate, 0.02 g/L of manganese chloride, 0.01 g/L of copper chloride, 0.01 g/L of zinc chloride, 0.02 g/L of magnesium sulfate, initial pH5.5, fermentation at 30° C. and 250 rpm. Take samples periodically to determine the content of glucose and xylitol. After 110 hours of fermentation, glucose is completely consumed, the content of xylitol is determined to be 148 g/L, and the conversion rate is 49.3%.

(15) Synthesis of Xylitol using Yeast Strain CGMCC No.18479 at 30° C. and 350 g/L of Glucose.

Inoculate the strains of recombinant yeast CGMCC No.18479 in a 2 L baffled flask containing 500 ml of fermentation medium with an initial opitical density (OD₆₀₀) of 0.8. Fermentation medium components: 350 g/L of glucose, 12 g/L of yeast cell powder, 5 g/L of peptone, 3 g/L of ammonium citrate, 0.02 g/L of manganese chloride, 0.02 g/L of copper chloride, 0.04 g/L of magnesium sulfate, initial pH5.5, fermentation at 30° C. and 250 rpm. Take samples periodically to determine the content of glucose and xylitol. After 138 hours of fermentation, glucose is completely consumed, the content of xylitol is determined to be 158 g/L, and the conversion rate is 45.1%.

(16) Synthesis of Xylitol using Yeast Strain CGMCC No.18479 at 35° C. and 300 g/L of Glucose.

Inoculate the strains of recombinant yeast CGMCC No.18479 in a 2 L baffled flask containing 500 ml of fermentation medium with an initial opitical density (OD₆₀₀) of 0.8. Fermentation medium components: 300 g/L of glucose, 10 g/L of yeast cell powder, 5 g/L of peptone, 3 g/L of ammonium citrate, 0.02 g/L of manganese chloride, 0.01 g/L of copper chloride, 0.02 g/L of magnesium sulfate, initial pH5.5, fermentation at 35° C. and 250 rpm. Take samples periodically to determine the content of glucose and xylitol. After 135 hours of fermentation, glucose is completely consumed, the content of xylitol is determined to be 122 g/L, and the conversion rate is 40.7%.

(17) Synthesis of Xylitol using Yeast Strain CGMCC No.18479 at 32° C. and 300 g/L of Glucose at Initial pH3.0.

Inoculate the strains of recombinant yeast CGMCC No.18479 in a 2 L baffled flask containing 500 ml of fermentation medium with an initial opitical density (OD₆₀₀) of 0.8. Fermentation medium components: 300 g/L of glucose, 10 g/L of yeast cell powder, 5 g/L of peptone, 3 g/L of ammonium citrate, 0.02 g/L of manganese chloride, 0.01 g/L of copper chloride, 0.02 g/L of magnesium sulfate, prepare the initial pH3.0 with citric acid, fermentation at 32° C. and 250 rpm. Take samples periodically to determine the content of glucose and xylitol. After 115 hours of fermentation, glucose is completely consumed, the content of xylitol is determined to be 142 g/L, and the conversion rate is 47.3%.

(18) Synthesis of Xylitol using Yeast Strain CGMCC No.18479 at 33° C. and 250 g/L of Glucose.

Inoculate the strains of recombinant yeast CGMCC No.18479 in a 2 L baffled flask containing 500 ml of fermentation medium with an initial opitical density (OD₆₀₀) of 0.8. Fermentation medium components: 250 g/L of glucose, 10 g/L of yeast extract, 5 g/L of steep powder, 3 g/L of ammonium citrate, 0.02 g/L of manganese chloride, 0.01 g/L of copper chloride, 0.01 g/L of zinc chloride, 0.02 g/L of magnesium sulfate, initial pH5.5, fermentation at 33° C. and 250 rpm. Take samples periodically to determine the content of glucose and xylitol. After 108 hours of fermentation, glucose is completely consumed, the content of xylitol is determined to be 121 g/L, and the conversion rate is 48.4%.

(19) Synthesis of Xylitol using Yeast Strain CGMCC No.18479 at 30° C. and 300 g/L of Glucose at Initial pH7.0.

Inoculate the strains of recombinant yeast CGMCC No.18479 in a 2 L baffled flask containing 500 ml of fermentation medium with an initial opitical density (OD₆₀₀) of 0.8. Fermentation medium components: 300 g/L of glucose, 10 g/L of yeast cell powder, 5 g/L of peptone, 3 g/L of ammonium citrate, 0.02 g/L of manganese chloride, 0.01 g/L of copper chloride, 0.02 g/L of magnesium sulfate, prepare the initial pH7.0 with sodium hydroxide, fermentation at 30° C. and 250 rpm. Take samples periodically to determine the content of glucose and xylitol. After 112 hours of fermentation, glucose is completely consumed, the content of xylitol is determined to be 132 g/L, and the conversion rate is 44%.

(20) Synthesis of Xylitol using Yeast Strain CGMCC No.18479 at 30° C. and 100 g/L of Fructose.

Inoculate the strains of recombinant yeast CGMCC No.18479 in a 2 L baffled flask containing 500 ml of fermentation medium with an initial opitical density (OD₆₀₀) of 0.8. Fermentation medium components: 100 g/L of fructose, 10 g/L of yeast cell powder, 5 g/L of peptone, 3 g/L of ammonium citrate, 0.02 g/L of manganese chloride, 0.01 g/L of copper chloride, 0.02 g/L of magnesium sulfate, initial pH5.5 with citric acid, fermentation at 30° C. and 250 rpm. Take samples periodically to determine the content of fructose and xylitol. After 120 hours of fermentation, fructose is still not completely consumed, the content of xylitol is determined to be 13 g/L, and the conversion rate is 13%.

(21) Synthesis of Xylitol using Yeast Strain CGMCC No.18479 at 30° C., 200 g/L of Glucose and 100 g/L of Fructose.

Inoculate the strains of recombinant yeast CGMCC No.18479 in a 2 L baffled flask containing 500 ml of fermentation medium with an initial opitical density (OD₆₀₀) of 0.8. Fermentation medium components: 200 g/L of glucose, 100 g/L of fructose, 10 g/L of yeast cell powder, 5 g/L of peptone, 3 g/L of ammonium citrate, 0.02 g/L of manganese chloride, 0.01 g/L of copper chloride, 0.02 g/L of magnesium sulfate, initial pH6.5, fermentation at 30° C. and 250 rpm. Take samples periodically to determine the content of glucose, fructose and xylitol. After 125 hours of fermentation, both glucose and fructose are completely consumed, the content of xylitol is determined to be 126.6 g/L, and the conversion rate of carbon sources, 300 g/L of glucose and fructose, is 42.2%.

(22) Synthesis of Xylitol using Yeast Strain CGMCC No.18479 at 30° C. and 100 g/L Glycerol.

Inoculate the strains of recombinant yeast CGMCC No.18479 in a 2 L baffled flask containing 50 ml of fermentation medium with an initial opitical density (OD₆₀₀) of 0.8. Fermentation medium components: 100 g/L of glycerol, 5 g/L of yeast cell powder, 3 g/L of peptone, 2 g/L of ammonium citrate, 0.02 g/L of manganese chloride, 0.01 g/L of copper chloride, 0.02 g/L of magnesium sulfate, initial pH5.5 with citric acid, fermentation at 30° C. and 250 rpm. Take samples periodically to determine the content of glycerol and xylitol. After 130 hours of fermentation, glycerol is still not completely consumed, the content of xylitol is determined to be 4.5 g/L, which may be due to that the transketolase gene is down-regulated by weak expression, and glycerol utilization efficiency is slowed down. In addition, the strain ery959ΔTKL12, whose transketolase gene is completely knocked out, can not synthesize xylitol from glycerol. Due to lack of transketone, the strain could not synthesize 5-p xylulose from glycerol, which is a precursor of xylitol, and thus no xylitol synthesized.

(23) Synthesis of Xylitol using Yeast Strain CGMCC No.18479 Utilizing Starch as Carbon Source.

Add 100 g of starch (from corncob) to 350 mL of cold water while agitating the water until it becomes starch milk, thus obtain 415 mL of starch milk (mass volume percentage of starch is 24%, i.e. 240 g/L). Heat to 90° C., then add 0.2 grams of thermoresistant α-amylase and stir until the starch is liquefied and clear. After cooling to 60 degrees, add 0.2 grams of medium temperature β-amylase and 0.1 grams of Pullulanase for saccharification, hold for 5 hours, then use for fermentation materials, add 3.5 grams of yeast cell powder, 2 grams of steep powder, 1.5 grams of ammonium citrate, 0.1 grams of magnesium sulfate, sterilize at 108° C. for 30 minutes, then allow to cool. Inoculate the strains of recombinant yeast CGMCC 18479 in this medium with an initial opitical density (OD₆₀₀) of 0.8, initial pH5.5, fermentation at 30° C. and 250 rpm. Take samples periodically to determine the content of glucose and xylitol. After 106 hours of fermentation, glucose in the fermentation medium is completely consumed, and the content of xylitol is determined to be 86 g/L, equivalent to the conversion rate of xylitol synthesized from starch is 35.8%.

In the above embodiments of xylitol synthesis by fermentation, the evaporated water must be replenished regularly to the initial weight during the fermentation process. Note down the weight of the fermentation bottle containing the fermentation liquid at the beginning of fermentation, and note down the weight every time you take a sample, and replenish the water with sterile water to the initial weight. The sample volume taken each time is 0.2 ml and diluted tenfold for HPLC determination of the content of carbon source materials (e.g. glucose, glycerol, fructose, etc.) and xylitol. The analytical column is SP0810 HPLC column of Shodex, refractive differential detector, pure water as mobile phase, flow rate is 1 mL/min, column temperature is 70 degrees.

(24) Synthesis of Xylitol using Yeast Strain CGMCC No.18479 in Fermenter.

Inoculate the strains of recombinant yeast CGMCC No.18479 in a 5 L-fermenter containing 3500 ml of fermentation medium with an initial opitical density (OD₆₀₀) of 0.8. Fermentation medium components: 300 g/L of glucose, 10 g/L of yeast cell powder, 5 g/L of peptone, 3 g/L of ammonium citrate, 0.01 g/L of manganese chloride, 0.01 g/L of copper chloride, 0.01 g/L of magnesium sulfate, 0.02 g/L of zinc chloride, initial pH6.5, fermentation at 30° C. with the initial speed of agitation is 300 rpm, and increase to 450 rpm when the cells growth reach to OD₆₀₀ above 3.0, and to 550 rpm when the OD₆₀₀ surpass 10.0. Take samples periodically to determine the content of glucose and xylitol. Steriled water should be added to compensate for water evaporated during fermentation. After 110 hours of fermentation, glucose is completely consumed, the content of xylitol is determined to be 152 g/L, and the conversion rate is 50.7%.

In each of the above steps, the fermentation medium is sterilized and cooled to room temperature before inoculating yeast strains.

(25) Purification of Xylitol from Fermentation Broth.

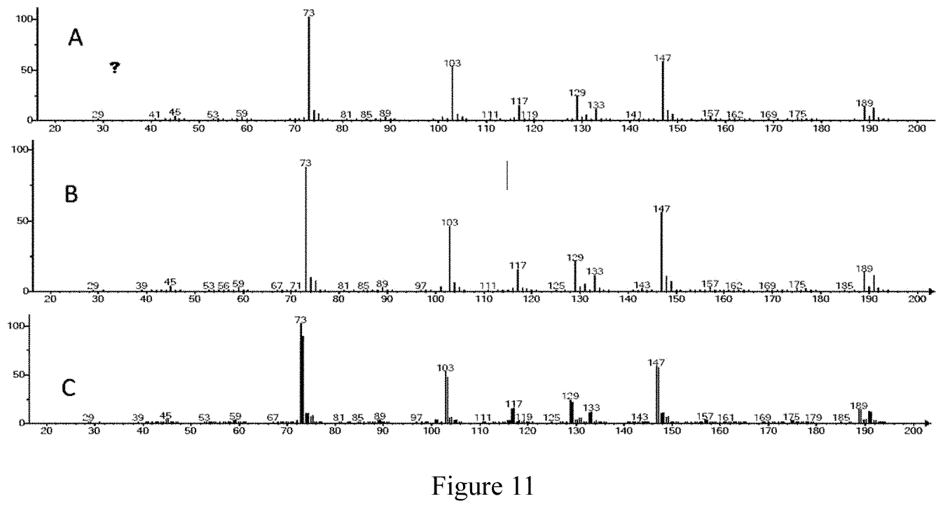

After fermentation, place the fermentation broth into a 500 ml-centrifuge tube and centrifuge at 6000×g for 20 minutes to obtain the clear xylitol-containing supernatant. Use 200 ml of pure water to suspend and wash the precipitated yeast cells to release the xylitol in the cells, and obtain the supernatant by centrifugation again. Collection of all fermentation supernatant, then transfer to a rotatory evaporating flask for evaporation and concentration. Measure the refractive index during this period, and stop evaporating when refractive index (soluble solid content) reached 68. Transfer the concentrated solution into a spherical flask and stir slowly with a magnetic agitator in a gradient cooler at 50 rpm. When the temperature decreased to 22° C., fine granular crystals began to appear. With the gradual decrease of temperature, the amount of crystallization gradually increased. At this time, increase the stirring speed to 80 rpm. Stop stirring when the amount of crystallization does not increase, and separate the crystals by centrifugation to obtain the crude products of xylitol. Redissolve in distilled water until refractive index is 45, then process successively the steps of ion exchange, decolorization, removal of ions and pigments, reconcentration, crystallization, centrifugation and drying, to obtain the refined products of xylitol. Analysis through GC-Mass to identify the xylitol isolated and purified from fermentation broth and the standard xylitol. FIG. 11 is the ion fragment peaks of xylitol synthesized by CGMCC No.18479 from glucose by fermentation in the present invention and standard xylitol, as well as the comparison between them. It is found that the ion fragments of the two are completely consistent, indicating that the product synthesized by the strain CGMCC No.18479 constructed by the method described in the present invention from glucose by fermentation is authentic xylitol.

The above description has described in detail certain exemplary embodiments. It is to be understood that the embodiments of the present invention are not to be limited to the above specific exemplary embodiments, that those skilled in the art, upon attaining an understanding of the foregoing, may readily conceive of alterations to, variations of, and equivalents to these embodiments within the scope of the claim, which does not affect the substance of the invention.

What is claimed is:

1. A construction method of recombinant Yarrowia lipolytica strain capable of synthesizing xylitol, comprising adopting the Yarrowia lipolytica strain capable of synthesizing erythritol as the host microorganism, by means of metabolic engineering or genetic engineering, to construct the recombinant Yarrowia lipolytica strain that synthesizes xylitol by fermentation with one carbon source or more carbon sources, including glucose, fructose, glycerol, or starch as carbon sources; the metabolic engineering or genetic engineering strategies include the expression of a gene encoding xylitol dehydrogenase and a gene encoding 5-p xylitol dehydrogenase in the cell of Yarrowia lipolytica from the host microorganisms, and the knockout or down-regulation of the transketolase gene in Yarrowia lipolytica.

2. The construction method as set forth in claim 1, wherein the host microorganism is the Yarrowia lipolytica strain whose genome contains DNA sequences with 97% or above identity with SEQ ID NO.3 sequence.

3. The construction method as set forth in claim 2, wherein the host microorganism is the Yarrowia lipolytica ery929 CGMCC No. 18478 that can synthesize erythritol.

4. The construction method as set forth in claim 1, wherein the expression of one or more of the following genes in the yeast Yarrowia lipolytica:
(1) the gene encoding 5-p xylulose phosphatase;
(2) the gene encoding xylitol transporter; and
(3) the gene encoding NADP transhydrogenase.

5. The construction method as set forth in claim 4, wherein the knockout or down-regulation of one or more of the following genes in the yeast Yarrowia lipolytica:
(1) mannitol dehydrogenase gene;
(2) arabinitol dehydrogenase gene;
(3) xylulose kinase gene; and
(4) 5-p ribulose isomerase gene.

6. A recombinant Yarrowia lipolytica strain capable of synthesizing xylitol obtained by using the construction method of claim 1 for construction of a recombinant Yarrowia lipolytica strain capable of synthesizing xylitol.

7. The recombinant Yarrowia lipolytica strain capable of synthesizing xylitol as set forth in claim 6, wherein the strain is Yarrowia lipolytica ery959ΔTKLΔMDHΔArDHΔRPIΔXKS1 CGMCC No. 18479.

8. A method of fermentation for xylitol synthesis using a recombinant Yarrowia lipolytica strain capable of synthesizing xylitol as set forth in claim 6, comprising the following steps:
S1. culturing the Yarrowia lipolytica strains in medium containing carbon, nitrogen, inorganic salts and water, and shaking or stirring, fermentation and culture at initial pH value of 3.0 ˜7.0 and temperature of 25 ˜35° C., then isolating the strains from the broth after fermentation to obtain xylitol-containing fermentation broth and yeast cells; and
S2. proceeding isolation and purification to the xylitol-containing fermentation broth and yeast cells to obtain xylitol.

9. The method of synthesizing xylitol by fermentation with the recombinant Yarrowia lipolytica strains capable of synthesizing xylitol as set forth in claim 8, wherein, in step S1, the carbon source in the medium is one or a mixture of glucose, fructose, glycerol and starch, and the carbon source concentration in the medium is 50-350 g/L; the nitrogen source in the medium is one or a mixture of peptone, yeast cell powder, yeast extract, corn steep powder, diammonium hydrogen phosphate, ammonium citrate and amino acids; the inorganic salt in the medium is one or a mixture of magnesium sulfate, manganese chloride, copper chloride and zinc chloride.

10. The method of synthesizing xylitol by fermentation with the recombinant Yarrowia lipolytica strains capable of synthesizing xylitol as set forth in claim 8, wherein the isolation and purification mentioned in step S2 include the isolation of yeast cells from the broth to obtain the clear fermentation broth containing xylitol, the concentration to obtain the concentrated solution rich in xylitol, the primary crystallization to obtain crude products of xylitol, which would obtain the refined products of xylitol through redissolution, ion exchange removal of ions, decolorization, concentration and secondary crystallization to the crude products, as well as the drying procedure.


SEQUENCE LISTING (79 sequences)

SEQ ID NO: 1 - DNA, 25 bp, Artificial Sequence
  Synthetic
tagtgcagatcttggtggtagtagc

SEQ ID NO: 2 - DNA, 24 bp, Artificial Sequence
  Synthetic
ctgcttcggtatgataggaagagc

SEQ ID NO: 3 - DNA, 1402 bp, Yarrowia lipolytica
tagtgcagatcttggtggtagtagcaaatattcaaatgagaactttgaagactgaagtggggaaaggttccgtgtgaacagcagttggacacgggtaagtcgatcctaaggggtggcataactgtcgcgtacggcccgataagggccttctccaaaagggaagccggttgaaattccggcacttggatgtggattctccacggcaacttaactgaatgtggggacggtggcacaagtcttggaaggagttatcttttctttttaacggagtcaacaccctggaattagtttgtctagagatagggtatcgttcaggaagaggggggcagctttgtcccctccgatgcacttgtgacgccccttgaaaacccgcaggaaggaatagttttcacgccaagtcgcactgataaccgcagcaggtctccaaggtgaacagcctctagttgatagaataatgtagataagggaagtcggcaaaatagatccgtaacttcgggataaggattggctctgggggttggtggatggaagcgtgggagaccccaagggactggcagctgggcaactggcagccggacccgcggcagacactgcgtcgctccgtccacatcatcaaccgccccagaactggtacggacaaggggaatctgactgtctaattaaaacatagctttgcgatggttctaaaacaatgttgacgcaaagtgatttctgcccagtgctctgaatgtcaaagtgaagaaattcaaccaagcgcgcgggtaaacggcgggagtaactatgctctcttaaggtagccaaatgcctcctcatctaattagtgacgcgcatgaatggattaacgagattcccactgtccctatctactatgtagcgaaaccacagccaagggaacgggcttggcagaatcagcggggaaagaagaccctgttgagcttgactctagtttgacattgtgaagagacatagggggtgtagaataagtgggagcttcggcgccggtgaaataccactacccttatcgtttctttacttatttagaaagtggaagtggtttaacaaccattttctagcattcctttccaggctgaagacattgtcaggtggggagtttggctggggcggcacatctgttaaaagataacgcagatgtcctaagggggactcaatgagaccagaaatctcatgtagaacaaaagggtaaaagtccccttgattatgattttcagtgtgaatacaaaccatgaaagtgtggcctatcgatcctttagttgttcggagtttgaacctagaggtgccagaaaagttaccacagggataactggcttgtggcagtcaagcgttcatagcgacatagctttttgatccttcgatgtcggctcttcctatcataacgaagcag

SEQ ID NO: 4 - DNA, 1092 bp, Scheffersomyces stipitis
atgactgctaacccatctttggttttgaacaagatcgacgacatctctttcgaaacttacgacgctccagaaatctctgaaccaactgacgttttggttcaagttaagaagactggtatctgtggttctgacatccacttctacgctcacggtagaatcggtaacttcgttttgactaagccaatggttttgggtcacgaatctgctggtactgttgttcaagttggtaagggtgttacttctttgaaggttggtgacaacgttgctatcgaaccaggtatcccatctagattctctgacgaatacaagtctggtcactacaacttgtgtccacacatggctttcgctgctactccaaactctaaggaaggtgaaccaaacccaccaggtactttgtgtaagtacttcaagtctccagaagacttcttggttaagttgccagaccacgtttctttggaattgggtgctttggttgaaccattgtctgttggtgttcacgcttctaagttgggttctgttgctttcggtgactacgttgctgttttcggtgctggtccagttggtttgttggctgctgctgttgctaagactttcggtgctaagggtgttatcgttgttgacatcttcgacaacaagttgaagatggctaaggacatcggtgctgctactcacactttcaactctaagactggtggttctgaagaattgatcaaggctttcggtggtaacgttccaaacgttgttttggaatgtactggtgctgaaccatgtatcaagttgggtgttgacgctatcgctccaggtggtagattcgttcaagttggtaacgctgctggtccagtttctttcccaatcactgttttcgctatgaaggaattgactttgttcggttctttcagatacggtttcaacgactacaagactgctgttggtatcttcgacactaactaccaaaacggtagagaaaacgctccaatcgacttcgaacaattgatcactcacagatacaagttcaaggacgctatcgaagcttacgacttggttagagctggtaagggtgctgttaagtgtttgatcgacggtccagaataa

SEQ ID NO: 5 - DNA, 1062 bp, Debaryomyces hansenii
atggctactaagcaaaacatcggtgttttcactaacccaaagcacgacttgtacgtttctgaaatcgaaactccagacgttggtgacttgtctgaagaagaagttttggttcacgttagatctactggtatctgtggttctgacgttcacttccaaaagcacggttgtatcggtccaactatggttgttgaagacgaacacatcttgggtcacgaatctgctggtgaagttttggctgttggtaacaaggttaagatcttgaaggttggtgacaaggttgctttggaaccaggtgttccatgtcacacttgtaagccatgtttgactggtaagtacaacggttgtgaaaacgttgaattctactctactccaccagttcacggtttcttgagaagatacatcaagcacccagctgctttctgtcacaagatcaacttgttgacttacgctcaaggtgctttgttggaaccattgtctgttgttttctgtggtatcagacacatcaacttgatcttgggtcaatctgttttggttttcggtgctggtccaatcggtttcgctactgctaaggctgctgaagctgctggtgcttacccaatcatggttactgacatcgaacaatctaagttggacttcatcaagaaggaaatcccatctgctatcactgctttggttaacggttctttgaaggaaaacgttgctaaggttactgacgacggtatcaacaacttcgacgttgctatcgaatgtactggtgttgaacaatctttggaattggctactcacgctttggacttcggtgctaagttgcacatcatcggtgttggtaaggaccaccaaaagttcccattcatgttgttgtctgttaaggaaatcaacatcactttccaatacagatacgctaacacttggccaactatcatcaagttggttgaagctggtatcatcaagttggacaacttggttactcacagattcaagttggaagacgctgttgacgctttcaagttggctggtaacccaaagtctggtgctatgaagatcttgatcgaagactcttaa

SEQ ID NO: 6 - DNA, 1038 bp, Agrobacterium sp.
atgaaggctttggttttggaagaaaagggtaagttgtctttgagagaattcgacatcccaggtaagttgggtccaaaggacgttagaatcagaactcacactgttggtatctgtggttctgacgttcactactacactcacggtaagatcggtcacttcgttgttcacgctccaatggttttgggtcacgaagcttctggtactgttatcgaaactggtgctgaagttgctcacttgaagccaggtgacagagtttgtatggaaccaggtatcccagacccaacttctagagcttctaagttgggtatctacaacgttgacccagctgtttctttctgggctactccaccaatccacggttgtttgactccagaagttatccacccagctgctttcacttacaagttgccagacaacgtttctttcgctgaaggtgctatggttgaaccattcgctatcggtatgcaagctgctttgagagctagaatccaaccaggtgacgttgctatcgttactggtgctggtccaatcggtatgatggttgctttggctgctttggctggtggttgtgctaaggttatcgttgctgacttggctcaaccaaagttggacatcatcgctgcttacgacggtatcgaaactgttaacatcagagaaagagacttgtctcaagctgttgctgacgctactgacggttggggttgtgacgttgttttcgaatgttctggtgctgctccagctgttttgggtatggctaagttggctagaccaggtggtgctatcgttttggttggtatgccagttgacccagttccagttgacatcgttggtttgcaagctaaggaattgagagttgaaactgttttcagatacgctaacgtttacgacagagctgttgctttgatcgcttctggtaaggttgacttgaagccattgatctctgctactatcccattcgaagactctatcgctggtttcgacagagctgttgaagctagagaaactgacgttaagttgcaaatcttgatgccacaataa

SEQ ID NO: 7 - DNA, 1044 bp, Gluconobacter oxydans
atggctcaagctttggttttggaaagaaagggtgaattgtctttgagagaaatcgacgttccagacgttttgggtccagacgacgttagagttgctatccacactgttggtatctgtggttctgacgttcactactacactcacggtagaatcggtcacttcatcgttgacgctccaatggttttgggtcacgaagcttctggtactgttactgaagttggttctagagttacttctttgcaagttggtgacagagtttgtatggaaccaggtatcccagacccaacttctagagcttctaagatgggtatctacaacgttgacccagctgttactttctgggctactccaccaatccacggttgtttgactccatctgttgttcacccagctgctttcacttacagattgccagaaaacgtttctttcgctgaaggtgctatggttgaaccattcgctatcggtgttcaagctgctgttaaggctgctttgaagccaggtgacacttgtttggttactggttgtggtccaatcggtttgatgactgctttggctgctttggcttctggtgctggtactgttttcatctctgacatcgctgctccaaagttgcaaatcgctggtcaatacaagggtttggttccattgaacgctaaggaagttagaccaagagacgctgtttctcaacaatgtggtgctgactggggtgttgacgttgttttcgaagcttctggtttcccaggtgcttacgacgacgttttctcttgtgttagaccaggtggtactgttgttttcgttggtatgccagttgaaaaggttccattcgacttggttgctgctcaagctaaggaaatcagaatggaaactgttttcagatacgctaacgtttacgaaagagctatcgctttgatctcttctggtaaggttgacttgaagccattgatctctgaaactttcccattcgctgaaggtatcgctgctttcgaaagagctgcttctgctagaccaactgacgttaagttgcaaatcaagttgccaggttaa

SEQ ID NO: 8 - DNA, 789 bp, Gluconobacter oxydans
atgtctaagaagttcaacggtaaggtttgtttggttactggtgctggtggtaacatcggtttggctactgctttgagattggctgaagaaggtactgctatcgctttgttggacatgaacagagaagctttggaaaaggctgaagcttctgttagagaaaagggtgttgaagctagatcttacgtttgtgacgttacttctgaagaagctgttatcggtactgttgactctgttgttagagacttcggtaagatcgacttcttgttcaacaacgctggttaccaaggtgctttcgctccagttcaagactacccatctgacgacttcgctagagttttgactatcaacgttactggtgctttccacgttttgaaggctgtttctagacaaatgatcactcaaaactacggtagaatcgttaacactgcttctatggctggtgttaagggtccaccaaacatggctgcttacggtgcttctaagggtgctatcatcgctttgactgaaactgctgctttggacttggctccatacaacatcagagttaacgctatctctccaggttacatgggtccaggtttcatgtgggaaagacaagttgaattgcaagctaaggttggttctcaatacttctctactgacccaaaggttgttgctcaacaaatgatcggttctgttccaatgagaagatacggtgacatcaacgaaatcccaggtgttgttgctttcttgttgggtgacgactcttctttcatgactggtgttaacttgccaatcgctggtggttaa

SEQ ID NO: 9 - DNA, 1095 bp, Candida maltosa
atgactccaaacccatctttggttttgaacaagatcgacgacatcactttcgaaaactacgacgctccagaaatcacttctccaagagacgttatcgttgaagttaagaagactggtatctgtggttctgacatccactactacgctcacggttctatcggtccattcgttttgagacaaccaatggttttgggtcacgaatctgctggtgttgttactgctgttggtaaggacgttactaacttgaaggttggtgacagagttgctatcgaaccaggtgttccatctagattctctgacgaaactaagtctggtcactaccacttgtgtccacacatggctttcgctgctactccaccagttaacccagacgaaccaaacccaccaggtactttgtgtaagtactacaaggctccagttgacttcttgttcaagttgccagaccacgtttctttggaattgggtgctatggttgaaccattgactgttggtgttcacggttgtaagttggctaacttgaagttcggtgaagacgttgttgttttcggtgctggtccagttggtttgttgactgctgctgttgctaagactatcggtgctaagagagttatggttgttgacatcttcgacaacaagttggaaatggctaaggaaatgggtgctgctactcacgttttcaactctaagactgacggtgactacgaagctttgatcaagaagttcgacggtgttcaaccatctgttgttttggaatgttctggtgctcaaccatgtatctacatgggtgttaagatcttgaaggctggtggtagattcgttcaaatcggtaacgctggtggtgacgttaagttcccaatctctgacttctctactagagaattgtctttgtacggttctttcagatacggttacggtgactaccaaacttctatcgacatcttggacaagaactacttgaacggtaaggaaaaggctccaatcaacttcgaattgttgatcactcacagattcaagttcaaggacgctatcaaggcttacgacttggttagagctggtaacggtgctgttaagtgtttgatcgacggtccagaataa

SEQ ID NO: 10 - DNA, 1089 bp, Trichoderma reesei
atggctactcaaactatcaacaaggacgctatctctacttctccatcttcttctacttctccagctacttaaccattgagatctggtagatctagaccatctagaactccaactacttcttcttctccatctactactagagcttctgctgctccaacttgtactactggttgtactgctccatctggtacttcttcttctagaactagatggtgttgggctacttctagaccagctccatcttctagatctgctagaccatctagagcttcttctccagctactgcttctccatcttctccagctactagagctggtggtgctccatctgctgctccagctaacactacttgtgctagaacttggtcttctccaccaagaagaagaactactgctccataaagagcttgtggtagaagaccaccaacttctgctacttcttgtagaactgcttgtagatgtagaagagctagataatcttctagatggccatggccatctactttgtcttctagaccagcttcttctagagcttctccatcttcttcttgggctccagctccatctgcttgttgtgctccaccatggccaagaagaactgctccaccaccattgtctgcttctacttcttgttctccatcttctactttgagagctgcttctgctagaagaactagaacttctagatctgcttctagattgagaactactcaaagaccatctagatcttggagagcttgtccagctgctccaacttcttaattgactccagctgctagatctagaagatctagaagagctttcacttcttctgcttgggctgctagaacttctagagctgcttgggctagagctacttctagatctccatcttggccatgtgcttctagaagataaagatctggtgctagatctgctactgctccagctactacttcttggagatcttcttggtctggtagaggtggttggacttctagatcttaattgagagctccatctgcttcttctagaagaagaagaagatctaagagatcttctttgggtagaccatctagattctaattgccaggtccaactagaagatgt

SEQ ID NO: 11 - DNA, 1152 bp, Neurospora crassa
atggctactgacggtaagtctaacttgtctttcgttttgaacaagccattggacgtttgtttccaagacaagccagttccaaagatcaactctccacacgacgttttggttgctgttaactacactggtatctgtggttctgacgttcactactggttgcacggtgctatcggtcacttcgttgttaaggacccaatggttttgggtcacgaatctgctggtactatcgttgctgttggtgacgctgttaagactttgtctgttggtgacagagttgctttggaaccaggttacccatgtagaagatgtgttcactgtttgtctggtcactacaacttgtgtccagaaatgagattcgctgctactccaccatacgacggtactttgactggtttctggactgctccagctgacttctgttacaagttgccagaaactgtttctttgcaagaaggtgctttgatcgaaccattggctgttgctgttcacatcactaagcaagctaagatccaaccaggtcaaactgttgttgttatgggtgctggtccagttggtttgttgtgtgctgctgttgctaaggcttacggtgcttctaaggttgtttctgttgacatcgttccatctaagttggaattcgctaagtctttcgctgctactcacacttacttgtctcaaagagtttctccagaagaaaacgctagaaacatcatcgctgctgctgacttgggtgaaggtgctgacgctgttatcgacgcttctggtgctgaaccatctatccaagctgctttgcacgttgttagacaaggtggtcactacgttcaaggtggtatgggtaaggacaacatcactttcccaatcatggctttgtgtatcaaggaagttactgcttctggttctttcagatacggttctggtgactacagattggctatccaattggttgaacaaggtaaggttgacgttaagaagttggttaacggtgttgttccattcaagaacgctgaagaagctttcaagaaggttaaggaaggtgaagttatcaagatcttgatcgctggtccaaacgaagacgttgaaggttctttggacactactgttgacgaaaagaagttgaacgaagctaaggcttgtggtggttctggttgttgttaa

SEQ ID NO: 12 - DNA, 1071 bp, Saccharomyces cerevisiae
atgactgacttgactactcaagaagctatcgttttggaaagaccaggtaagatcactttgactaacgtttctatcccaaagatctctgacccaaacgaagttatcatccaaatcaaggctactggtatctgtggttctgacatccactactacactcacggtagaatcgctaactacgttgttgaatctccaatggttttgggtcacgaatcttctggtatcgttgctttgatcggtgaaaacgttaagactttgaaggttggtgacagagttgctttggaaccaggtatcccagacagattctctccagaaatgaaggaaggtagatacaacttggacccaaacttgaagttcgctgctactccaccattcgacggtactttgactaagtactacaagactatgaaggacttcgtttacaagttgccagacgacgtttctttcgaagaaggtgctttgatcgaaccattgtctgttgctatccacgctaacaagttggctaagatcaagttcggtgctagatgtgttgttttcggtgctggtccaatcggtttgttggctggtaaggttgcttctgttttcggtgctgctgacgttgttttcgttgacttgttggaaaacaagttggaaactgctagacaattcggtgctactcacatcgttaactctggtgacttgccacacggtgttactgttgactctgttatcaagaaggctatcggtaagaagggtgctgacgttgttttcgaatgttctggtgctgaaccatgtgttagagctggtatcgaagtttgtaaggctggtggtactatcgttcaagttggtatgggtcaagaagaaatccaattcccaatctctatcatcccaactaaggaattgactttccaaggttgtttcagatactgtcaaggtgactactctgactctatcgaattggtttcttctagaaagttgtctttgaagccattcatcactcacagatactctttcaaggacgctgttgaagctttcgaagaaacttctcaccacccattgaacaacatcaagactatcatcgaaggtccagaataa

SEQ ID NO: 13 - DNA, 1074 bp, Yarrowia lipolytica
atgtcttctaacccatctttcgttttgagaaagccattggacttggttttcgaagacagaccagacccaaagatccaagacccacactctgttaaggttgctgttaagaagactggtgtttgtggttctgacgttcactactacttgcacggtggtatcggtgacttcatcgttaaggctccaatggttttgggtcacgaatctgctggtgaagttgttgaagttggtccagaagttaaggacttgaaggttggtgacagagttgctttggaaccaggtgttccatctagattgtctcaagaatacaaggaaggtagatacaacttgtgtccatgtatggttttcgctgctactccaccatacgacggtactttgtgtagacactacatcatcccagaagacttctgtgttaagttgccagaccacgtttctttggaagaaggtgctttggttgaaccattgtctgttgctgttcactgtaacaagttggctaagactactgctcaagacgttgttatcgttttcggtgctggtccagttggtttgttggctgttggtgttgctaacgctttcggttcttctactatcgtttgtgttgacttggttccagaaaagttggaattggctaagaagttcggtgctactcacactttcgttccaactaagggtgactctccaaacgaatctgctgacaagatcagagctttgatcaagggtgctggtttgtctgactctccaaacgttgctttggaatgtactggtgctgaaccatctatccaaactgctgtttctgttttggctacttctggtagattggttcaagttggtatgggtaaggacgacgttaacttcccaatcactaagtgtatcgttaaggaaatcactgttttgggttctttcagatactgtcacggtgactacccattggctgttcaattggttgcttctggtaagatcgacgttaagaagttggttactaacagattcactttcaaggaagctgaacaagcttacaagactgctgctgaaggtaaggctatcaagatcatcatcgacggtccagaagaagaataa

SEQ ID NO: 14 - DNA, 1053 bp, Clostridioides difficile
atgaaggctgctgttttgcacggtactaacgacatgagattcgaagacatcgaaatcaagccatgtgaatctgacgaagttaagatcaaggttatggctgctggtatctgtggttctgacccaccaagagttttgaagcactggaagtacccagttccagctatcccaggtcacgaattctctggtgttatcgctgaagttggtaaggacgttaagaacgttaaggttggtgacagagttgttgctatcccattcatcccatgtaacgaatgtgaatactgtaagagaggtttgttctctttgtgtgacgaccacggtatgttgggtgctaagtctttcggtgctttcgctgaatacgttaacatcaaggctactaacgttttgccaatcggtgacatggacttcgaagacgctgctatgatcgaaccattggctgttgctatgcacggtgttttgaacatcggtgttcaagttggtgacactgttgctgttatgggttctggtactatgggtcaattggttatccaaggtttgaagatcgctggtgctggtactatcatcgctgttgacatctctgacaacaagttgagagaatctaaggaattgggtgctgacatcatcatcaacgctaaggacatcaacccagttgaaaagatcaaggaattgactggtggtaagggtgttgacatcgctttggaatgtgctggttctaagatcactcaagaacaatgtttgttgatcactaagaagaagtctaagatcggtttcttgggtatcgcttactctgacatcactttgtctgaagaagctttcgaaaacatcttcagaaaggaattggaattgaagggtttctggaactcttactctgctccattcccaggtcaagaatggactaagggtatcaacttggttaacgaaggtaagatcaagttgaaggaaatggtttctcacagattctctttggaagacacttacaaggctttcgaaatgatcagagacagaaaggaagaattcaacaagatcttgatcttgccacaaggtgttgaaaagtaa

SEQ ID NO: 15 - DNA, 1053 bp, Clostridioides difficile
atgaagtctgttagattctacggtatcagagacactagagttgaagacgttgacgttccaaagatcttggaaaaggacgacgttatcatcaaggttaaggttgctggtatctgtggttctgacatctctaagtactctaagactggtccacacatggttggtgaaatcttgggtcacgaattctctggtgaagttgctcaagttggtaaggaagttagatctttcaagatcggtgacagagttgctgtttgtccagctatgccatgtttcgaatgtgacgaatgtaagaagggtttgtactctagatgtaacaacgttgctatcatcggtaacaaggaattgggtggttgtttcgctgaatacactaaggttaaggaaagaaacttgatcaagatcccagacgaaatctcttacgaaactgctgctgctttggaaccagtttgtatcgctggtcacggtttgttcagatctgaagctaaggttggtgacactgttgttgttttgggtactggtccaatcggtttgttctctatccaatgggctaagatcttcggttctactaagatcatcgctgttgacgttttcgacgaaaagttggacttggctaaggaattgggtgctgacatctgtatcaacgctaaggaaaagaacatcgttgaagaaatcaagagattgactgacggtgacggtgctgacatcgttatcgaatctgctggtactccattgacttgtggtcaagttttgttgttggctaagaagggtggtactgttttgtacgctggtgttccatacggtgacgttgctttgactagagaacaattcgaaaagatcgttagatctgaattgactgttaagggtacttggttcggtaactctttcccattcccaggtaaggaatggtctgctggtttgtaccacatgcaaaagggtgacatgaacgttgaaaagttggttactcacagaatcaacttggaagaagctccagcttacttcgaaaaggtttacaagagagacatcttcttcggtaagatcatgatcaacatcgacaactaa

SEQ ID NO: 16 - DNA, 1059 bp, Clostridioides difficile
atgggtaacaagatgagagcttctgttttgtacaacgttggtgacgttagatacgaaatggttgacatcccagaaatcactgacactcaagttttggttaacgttaagtacgttggtatctgtggttctgacttgccaagatctatggtttctggtttgtctggtaacactaagtacccattgatcttgggtcacgaattctctggtgaagttgttaagatcggtgaaaaggttaagcacatcaacgttggtgacagagttgctgttgctccattggttccatgtggtaagtgtgactactgtaacgaaggtaacttcggtttgtgtgacgactacaacatcatcggtactagagttaacggtgctttcgctgaatacgttagagttccagaagaacacatcttgaagttgccagacactttggactacgaaactgctgctggtatcgaaccagctactatcgcttaccacggtatctctaagtctaacatcagagttggtgactctgttgttgttttgggttgtggtccaatcggtcaattcgttatccaatgggctaaggttttcggtgcttctaagatcatcgctgttgacatcttcgacgaaaagttggaattgtctaagttgttgggtgctaactacatcttgaactctaaggaagttaacgttatcaaggaaatcaagaagatcactaacggtggtgctgacgttgttatcgaaactgctggttctagattcactcaagaacaatctttgttcgttgctaagaagagaggtaacatcgttttcgttggtatctctcacactgaattgccattgtctgctgacgctactgaatgtatcttgagaggtgaattgactttgaagggttcttggaactcttacacttctccatacccaggtagagcttggactgctactttggacttcatggaaaagggtgacatcatcttcaagccaatgatctctgacaagatcggtttgaacgaagttggtgacttcttgtctaagatgtctaagagagaaatcaacttcaacaagatcttggttgaaatctaa

SEQ ID NO: 17 - DNA, 1050 bp, Lactobacillus rhamnosus
atgaaggcttctatgttggaagacttgaacaagttctctgttaaggaaatcgacatcccatctccaaagaaggacgaagttgttgttaaggttatggctgctggtacttgtggttctgactctcacaagatgatctctggttggaagtacggttacccagctgttatgggtcacgaattctctggtatcgttactcaattgggtgaaaacgtttctaacgtttctgttggtcaacacgttgctgttgctccattcatcccatgtttcaagtgtcactactgtcaaatcggtttgttccaaatgtgtgaaaactactctatgttgggtcaacaaaagttcggtggtttcgaacaatacgtttctgttccagctagaaacgttttggacatcggtaagatgtctttcgaagaaggtgctttgatcgaaccaatggctgttgctgctcacgctgttatgggtatcaagccagaattgggtgacactgttgctgttttcggtttgggtactgttggtgacttggttgttagattgttgatctcttctggtgctactaacgttatcggtatcgacatcgacgaccaaaagttggaaaagggtttggacgaaggttgtactcacgttatcaactctgctaaggaatctttggaagaaaagatcatggaatacactgacggtttgggtgttgacatctctatggaatgtgctggttctaagatcactgaagaacaaactttgttggttactaagagaagaggtaaggttggtttcgttggtatcgcttactctgacgttttgttgcaccaaaaggctttcgaaaacatcttcagacacgaattgactgttactggtttctggaactcttactctgctccattcccaggtagagaatggactaactctatccaattggttaacagaggtagaatcaagatcaaggacttgatcactcacagattcgaattggaagacatgcaaaaggctttcaacatgatcactactagatctgaatctttcaacaaggttatgttcttcccaaacggtatcaactaa

SEQ ID NO: 18 - DNA, 753 bp, Lactobacillus paracasei
atgccagacaactactctactgactctgttttcttgtcttctggttacgacggtatcgctcaatctgacttggttcaaccagacagagctgttgttgctttgccagaaaacatcccagacgaaatcgctatcttgactgaagtttctactgttggttaccacgcttcttctcacgttgctgacactttggctaagccaggttgtagagttgctttgttcggtgacggtccagttggttacatggctgctgctgttttgcactacatcagaggtatcgacaaggaccacttgactgttttcggtgctatcccagacagattgaacgaattcgacttcgctaacaaggaattggttactgaatacgacttcgaccacgctggtgaacaattcgacgttatcttcgaagctactggtggtaacttctcttcttctgctatcaacgaaggtatcaaggttatctctagaactggtaagttcgttttgatgggtgtttctgaagacttggttccaatcgacactagagacatcttggaaaagggtttgactttctacggtacttctagatctactactccagacttcgaagctgttgttaaggctatgtctcaatctcaagactaccaagacactttgagaaagttgttgccaaagaacgaaactgttatcaagaacgcttctgacttgaacaaggctttcgaagctatcgttgcttctaaggcttggtacaaggctgttttgaagttcgaatggtaa

SEQ ID NO: 19 - DNA, 1152 bp, Lactobacillus casei
atgatgagagctatcatctgtaacgttaagactgttactaagaacactgaagctgacatcatgttgaagaacccagacgtttctagaaagatcgcttctaagtcttacagattgatcaagccaggtgacatcgaagaagttaacttgcaacacgaattgagaccaggtttggttgacatccaaccattgatggcttctgtttgtcacgctgacgacagatacttcgctggtaagagaagaccagaagctttggctaagaagttgccaatggctttgttgcacgaaggtatcggtactatcaaggaatctatgtctgacaagttcaaggttggtcaaagagttgttatcgttccaaacgttccaggttacatgttgagaggtgaaaagaagactgacactgttccagacaactactctactgactctgttttcttgtcttctggttacgacggtatcgctcaatctgacttggttcaaccagacagagctgttgttgctttgccagaaaacatcccagacgaaatcgctatcttgactgaagtttctactgttggttaccacgcttcttctcacgttgctgacgctttggctaagccaggttgtagagttgctttgttcggtgacggtccagttggttacatggctgctgctgttttgcactacatcagaggtatcgacaaggaccacttgactgttttcggtgctatcccagacagattgaacgaattcgacttcgctaacaaggaattggttactgaatacgacttcgaccacgctggtgaacaattcgacgttatcttcgaagctactggtggtaacttctcttcttctgctatcaacgaaggtatcaaggttatctctagaactggtaagttcgttttgatgggtgtttctgaagacttggttccaatcgacactagagacatcttggaaaagggtttgactttctacggtacttctagatctactactccagacttcgaagctgttgttaaggctatgtctcaatctcaaggttaccaagacactttgagaaagttgttgccaaagaacgaaactgttatcaagaacgcttctgacttgaacaaggctttcgaagctatcgttgcttctaaggcttggtacaaggctgttttgaagttcgaatggtaa

SEQ ID NO: 20 - DNA, 1026 bp, Lactobacillus plantarum
atgttgaaccaagtttacagattggttgacccaagacaattcgaagttcaaactgttgctgaagaaatcactaacaacgacatcatcgttagaccaagattcttgtctgtttgtcacgctgacactagatacttcactggtcaaagaccacaagctactttgagacaaaagttgccaatggctttgatccacgaaggtgttggtgaagttgttaaggacccacaagacaagttcaagccaggtactttggttgctatggttccaaacactccattcgaaactgacccaatcatcaaggaaaactacttgccatcttctaagttcagatcttctggttacgacggtttcatgcaagaatacgtttctttgcacagagacagagctatcgttgttccagacaacttcgaccaccaaatgtctgctttcatcgaaatggtttctgttggtgttcacgctttgactcaattggaaggtgttatggacgctgacagaaaggttatcggtatctggggtgacggtaacttgggtttcatcactgctactttggttaagcaaatcttcccagactctcaattgatgatcttcggtagacaccaatctaagttggactacttctctttcgctgacaagacttacttggttgacgacatcccaaacgacttgaaggtttctcaagctttggaatgtactggtggtagaggttctgaatctgctatcgctcaaatcatccaacacatcagaccaatgggtactgctatcttgatgggtgtttctgaagacccagttggtatcgacactagatctgttttggctgaaggtttgactttgagaggtgtttctagatctggtagagctgacttccaaagagctgttgacatcttgactgactctccagttactagagaaagattgcaaaacttggttggtttcactagaaaggtttctactatccaagacatcactgacttcttcgaaggtgctttgactaactactggggtaaggctgttatggaatgggacgtttaa

SEQ ID NO: 21 - DNA, 747 bp, Kluyveromyces marxianus
atgccattgatcactgttaactacttgttgttcgacttggacggtactttggtttcttctactgacgctgctgaccaaacttggaaggactactgtgaaaagcacggtgtttcttacgaagaattgtctaagactgttcacggtactagaactgctgaaactttggctaagtacttcccaaacgttgacaacactgacaacaaggctgttaaggaattggaatgttctatcgctaacaactacaaggaattggtttctttggttccaggtgcttctgacttgttgatctctttggacagaccaactggttctttgccaggtgaagttttcaagcacagaaagtgggctatcgttacttctggtactccatgggttgctgacgcttggttcgaccacatcttgaagtctgttggtaagccagaagttttgatcactgctaacgacgttacttctggtaagccagctccagacggttacttgttggctgctcaaagattgaaggaaaagtggcaagacgacagaaaggacttgagaactgttgttttcgaagacgctccagttggtgttagagctggtaaggcttctggttctatcgttgttgctttgactactacttacgacaaggaatctttgttcgaagctggtgctgactacgttgttgaagacttgactcaagtttgtgttagatctaacactactgcttctactgttttgatcatcactgacccaatggaaagagacgaataa

SEQ ID NO: 22 - DNA, 741 bp, Saccharomyces cerevisiae
atggctgaattctctgctgacttgtgtttgttcgacttggacggtactatcgtttctactactgttgctgctgaaaaggcttggactaagttgtgttacgaatacggtgttgacccatctgaattgttcaagcactctcacggtgctagaactcaagaagttttgagaagattcttcccaaagttggacgacactgacaacaagggtgttttggctttggaaaaggacatcgctcactcttacttggacactgtttctttgatcccaggtgctgaaaacttgttgttgtctttggacgttgacactgaaactcaaaagaagttgccagaaagaaagtgggctatcgttacttctggttctccatacttggctttctcttggttcgaaactatcttgaagaacgttggtaagccaaaggttttcatcactggtttcgacgttaagaacggtaagccagacccagaaggttactctagagctagagacttgttgagacaagacttgcaattgactggtaagcaagacttgaagtacgttgttttcgaagacgctccagttggtatcaaggctggtaaggctatgggtgctatcactgttggtatcacttcttcttacgacaagtctgttttgttcgacgctggtgctgactacgttgtttgtgacttgactcaagtttctgttgttaagaacaacgaaaacggtatcgttatccaagttaacaacccattgactagagcttaa

SEQ ID NO: 23 - DNA, 741 bp, Saccharomyces cerevisiae
atgccacaattctctgttgacttgtgtttgttcgacttggacggtactatcgtttctactactactgctgctgaatctgcttggaagaagttgtgtagacaacacggtgttgacccagttgaattgttcaagcactctcacggtgctagatctcaagaaatgatgaagaagttcttcccaaagttggacaacactgacaacaagggtgttttggctttggaaaaggacatggctgacaactacttggacactgtttctttgatcccaggtgctgaaaacttgttgttgtctttggacgttgacactgaaactcaaaagaagttgccagaaagaaagtgggctatcgttacttctggttctccatacttggctttctcttggttcgaaactatcttgaagaacgttggtaagccaaaggttttcatcactggtttcgacgttaagaacggtaagccagacccagaaggttactctagagctagagacttgttgagacaagacttgcaattgactggtaagcaagacttgaagtacgttgttttcgaagacgctccagttggtatcaaggctggtaaggctatgggtgctatcactgttggtatcacttcttcttacgacaagtctgttttgttcgacgctggtgctgactacgttgtttgtgacttgactcaagtttctgttgttaagaacaacgaaaacggtatcgttatccaagttaacaacccattgactagagactaa

SEQ ID NO: 24 - DNA, 687 bp, Komagataella phaffii
atggtttctatcccatgtgacttgtgtttgttcgacttggacggtactttggttttgtctactaaggctatcgaaaagggttgggaatctgttttctctgaatacaacgttaactacaacatggaagaattcttgcaaaacaaccacggtgttagaactggtgactctttcgacagatggttgccacaaatcgacaacactaactctaaggctggtgacgaattcgaaaagagaatctctatcgaatacgctgacttggctgaaccagttccaggtgctccacaattgttgaactctatcccaaaggaccactggttggttgttacttctggtactccattgttggctaacggttggttctctaaggttttggctaagttcggtgttactaagccagaaatcttcgttactggtcaatctgtttctaacggtaagccacacccagaaccatacttgaagggtttggctttgtggactgaaaagtacggtaagaagccagctcacccaatcgttttcgaagacgctccaaacggtatcaaggctggtactgcttctggttgtactgttatcggtatcgcttcttctttcggtaaggaagttttgcaagctgctggtgctacttacgttgttcaagacttgtctcacgttaagttccacgacaacactttggacatcgacaacttgtaa

SEQ ID NO: 25 - DNA, 813 bp, Lactobacillus kunkeei
atgtctgaaatcaagttgatcgctatcgacatcgacggtactttgttgaacgaagaaaacatcttggctcaagaaactatcgacgctgttactgaagctagaaacaacggtatcaaggttgttttgtgtactggtagaccattgactggtgttaagccatacttgaagaagttgaacatctctggtaacgacgaatacgctatcactttcaacggtgctcaagttcaagacgctgacgctaacatcatcgaaaagttcgacttggactacaacgacttcgttggtttggaaaagttgtctcacaagttgaacactaacttccaaatcgaaactactgactacatctacactactaacagagacttgtctccatactctgttgctgaatcttacttggttagaatgccaatcagagttagagaaccacaagaaatcactgacgaaactgaaatcgttaaggctatgttgatcgctgacccagacatcatcgacaaggctatcccaaacatcccagaaaacttcttggaccacttgactatggttagatctgaaccagttttcttggaattcgttaaccaaaaggcttctaagggtgctgctttgtctaagttggctactagattgggtttcaacgctgaaaacgttatggctatcggtgaccaaggtaacgacatctctatggttacttacgctggtactggtgttgctatgggtaacgctgctgacgacttgaagaaggttgctaacaaggttactaagactaacaaggaaaacggtgttgcttacgctatcagaaacttcgctttgaagtaa

SEQ ID NO: 26 - DNA, 777 bp, Lactobacillus paracasei
atgaagtacaagggttacatgatcgacttggacggtactatctacagaggtaaggaaagaatcccagctgctaaggacttcgttgaaagattgcaagctgctcaaatcccattcttgttcttgactaacaacactactaagtctccagaagacgttgttaagaacttggctgaaaaccacgacatccacgttcaaccagctcaagtttacactccagctttggctactgctgcttacttgactgacttgaaccacggtgacgttactggtaagtctatctacatcatcggtgaattgggtttgaagcaagctgttttggacactggtttgagattgaacgaagttgacccagactacgttgttgttggtttggactacgacgttacttaccacaagttcgaattggctactttggctatcaagagaggtgctaagttcatcggtactaacgctgacactaacttgccaaacgaaagaggtttggttccaggtgctggttctttgatcgctttggttgaaagatctactcaacaaagagctttctacatcggtaagccagaaccaactatcatggaaaaggctttgaagaagatgggtttgccaaaggaagctgttgctatggttggtgacaactacaacactgacatcaaggctggtttgaacgctggtatcgacactatcttggtttacactggtgtttctactagagactacgtttctaagcaagttcaccaaccaactcaccaaatcgacgctttgactgactgggaagtttaa

SEQ ID NO: 27 - DNA, 816 bp, Lactobacillus plantarum
atggaaaacatcaagatgatcgctatcgacatcgacggtactttggttaactctaagaagcaagttactttgagagttaagcaagctatcaagatggctaagaagaagaagatcaaggttgttatctgtactggtagaccattgactggtgttaaggctttgttgcaagaattggaattggacgctcaagacgaccaatacgttgtttgtttcggtggtgctgctacttacactacttctggtgaattgatcgacgaaagaccaatctcttacgaagactacatcgacttggaagctttggctagaaagttgagattgcacttccacactgtttctgaagacagattgtacactgctgacagaaacatcggtgactacactttgtacgaagctaacttggtttctatgggtatctcttacagaactccagaagaaatgagaaacatcaagttgatcaagtctatgtacgttgacgaaccagaagttttggacgctgctatcaagcaacaaaagttgttcgaaccattgaagaagcaagttactttcactaagtctgctccattctactacgaagctaacgctaacggtgtttctaagggtaacgctttgcaagttttgtgtgaaaagttgtctttgactgctgctaacgttatggctatcggtgacgaagctaacgacttgtctatgatcaagttcgctggtcacggtgttgctatgggtaacgctatcccagaagttaagcaagttgctgacgaaatcactgttgacaacgaacacgacggtgttgctaaggctatcgaagctatcactagataa

SEQ ID NO: 28 - DNA, 822 bp, Lactobacillus fermentum
atgtctatcaagttgatcgctatcgacatcgacggtactttgatcaacgaccaattggaaatcactgaaaagactaaggaaactttgcaaaaggctactgctcaaggtatcaaggttgttttgtgtactggtagaccaatgactggtgttcacaagtacttggaccaattgggtatcaacaacttggctgaccaatacgttatctctttcaacggtgctttggctcaaactacttctggtcaagttatctctcaattcactttgccattcgaaaagttggttgacttgtctgctgttgctttgaaggctgacgttcacttgttggctgaaactgctgacgctatgtacgttttgaaccaagacatctcttcttacgctgtttacgaatcttctttggtttctttgccaatcacttacaagtctatcgaccaattgaacactatcaagaacgacttggttatctctaagttgatgatcactgacgaaccagctgctatcgacggtttctctgctaagttgactgctccaatcaagagagctttcaacatcgttagatctgaaccatactacttggaattcgttaacccatctgcttctaagggtgctgctttggcttctttgggtcaagaattgggtgttgctagaactgaaatgatggctatcggtaacgctcaaaacgacgaatctatgatcacttacgctggtatcggtgttgctatgtctaactctatcccatctactatccaattggctgacgaattggttgctgacaacaaccacgacggtgttgctgaagctgttgaaaagttcgctttggcttaa

SEQ ID NO: 29 - DNA, 891 bp, Aspergillus niger
atgtctccagaaactcaagaatctggtccattcgcttctcacatcttcgctggtgttttgttggacttcgacggtactatcatcgactctactgaaggtgaatctactatcccaatccaaaacccaaagagaatctaacacttgccatctgacaacagagaattggaaaaggtttacccacaccaacacatcttgcaagctactttgttgactactactgaatctgctatgaactaagcttctactatcatgaagtcttctgctccaagaactggtgacggtgcttctatgtcttacaacaactaaatcccacaaagaccaactggtaacgtttgtagattgactttgccatctatctctcacccaaagtcttaattgacttaatctttgtctttggacgtttctgaaatggaatctcaaatcccaactttgtctaagactccagctgttgaaatcccaggtgctagaaacttgttggaatctttggctaagttccacatcccacacgctatcgttacttctggtactaaggctttgttgtctggttggttgaacgttttgcaattgccacaaccacaacacgttactgttgctgaagacgttactttgggtaagccagacccagaaggttacagaaagggtaaggaaaagatcttggctggtagagttaacgacgacaacggttctaaggacgttttggttgttgaagacgctccagctggtatcagagctggtaaggctgctaactgtaaggttttggctgttgctactactcactctgttgacgttttgaagagagctggtgctgactgggttgttagagacttgagattcgttggtgttgaaagatgtgaaagaggtttcgaagttcacttctctggtttgttgtaa

SEQ ID NO: 30 - DNA, 702 bp, Aspergillus japonicus
atggctcaatctttccaatctgctggtagaactgaaagacacgctttcgctggtgttttgttggacttcgacggtactatcatcgactctactgaagctatcgttgaaaactggactagagttgctgctgaattgggtttggaccacagagacatcttgagagcttctcacggtagaagatctatcgacgttttgaaggaattggacccaactaaggctaactgggaatacgtttctgctatggaagctagaatcccattgttgtcttctactccagctgttgaaatcgctggtgctagaagattgttggaacaattgaaccactactctatcccacacgctatcgttacttctggttctaaggctttgttggacgcttggttgtctatcttgcaattgccaagagctatgaaggctactactgctgaagacgttaagatcggtaagccagacccagaaggttacagaatggctaagaagaagttgttgcaacacagatctggtgaaggtgaagttttggttatggaagacgctccagctggtatcgttgctggtaaggctgctggttgtaaggttttggctgttactactactcacactgttcaacaattgaaggaagctggtgctgactgggttgttagagaccacagattcgttgacttcgaagctccagctggttctggtgacatggttttcagataa

SEQ ID NO: 31 - DNA, 819 bp, Bacillus subtilis
atgagaatcatggcttctcacgacactccagtttctccagctggtatcttgatcgacttggacggtactgttttcagaggtaacgaattgatcgaaggtgctagagaagctatcaagactttgagaagaatgggtaagaagatcgttttcttgtctaacagaggtaacatctctagagctatgtgtagaaagaagttgttgggtgctggtatcgaaactgacgttaacgacatcgttttgtcttcttctgttactgctgctttcttgaagaagcactacagattctctaaggtttgggttttgggtgaacaaggtttggttgacgaattgagattggctggtgttcaaaacgcttctgaaccaaaggaagctgactggttggttatctctttgcacgaaactttgacttacgacgacttgaaccaagctttccaagctgctgctggtggtgctagaatcatcgctactaacaaggacagatctttcccaaacgaagacggtaacgctatcgacgttgctggtatgatcggtgctatcgaaacttctgctcaagctaagactgaattggttgttggtaagccatcttggttgatggctgaagctgcttgtactgctatgggtttgtctgctcacgaatgtatgatcatcggtgactctatcgaatctgacatcgctatgggtaagttgtacggtatgaagtctgctttggttttgactggttctgctaagcaaggtgaacaaagattgtacactccagactacgttttggactctatcaaggacgttactaagttggctgaagaaggtatcttgatctaa

SEQ ID NO: 32 - DNA, 2010 bp, Saccharomyces cerevisiae
atgtctaacccacaaaaggctttgaacgacttcttgtcttctgaatctgttcacactcacgactcttctagaaagcaatctaacaagcaatcttctgacgaaggtagatcttcttctcaaccatctcaccaccactctggtggtactaacaacaacaacaacaacaacaacaacaacaacaactctaacaacaacaacaacggtaacgacggtggtaacgacgacgactacgactacgaaatgcaagactacagaccatctccacaatctgctagaccaactccaacttacgttccacaatactctgttgaatctggtactgctttcccaatccaagaagttatcccatctgcttacatcaacactcaagacatcaaccacaaggacaacggtccaccatctgcttcttctaacagagctttcagaccaagaggtcaaactactgtttctgctaacgttttgaacatcgaagacttctacaagaacgctgacgacgctcacactatcccagaatctcacttgtctagaagaagatctagatctagagctacttctaacgctggtcactctgctaacactggtgctactaacggtagaactactggtgctcaaactaacatggaatctaacgaatctccaagaaacgttccaatcatggttaagccaaagactttgtaccaaaacccacaaactccaactgttttgccatctacttaccacccaatcaacaagtggtcttctgttaagaacacttacttgaaggaattcttggctgaattcatgggtactatggttatgatcatcttcggttctgctgttgtttgtcaagttaacgttgctggtaagatccaacaagacaacttcaacgttgctttggacaacttgaacgttactggttcttctgctgaaactatcgacgctatgaagtctttgacttctttggtttcttctgttgctggtggtactttcgacgacgttgctttgggttgggctgctgctgttgttatgggttacttctgtgctggtggttctgctatctctggtgctcacttgaacccatctatcactttggctaacttggtttacagaggtttcccattgaagaaggttccatactacttcgctggtcaattgatcggtgctttcactggtgctttgatcttgttcatctggtacaagagagttttgcaagaagcttactctgactggtggatgaacgaatctgttgctggtatgttctgtgttttcccaaagccatacttgtcttctggtagacaattcttctctgaattcttgtgtggtgctatgttgcaagctggtactttcgctttgactgacccatacacttgtttgtcttctgacgttttcccattgatgatgttcatcttgatcttcatcatcaacgcttctatggcttaccaaactggtactgctatgaacttggctagagacttgggtccaagattggctttgtacgctgttggtttcgaccacaagatgttgtgggttcaccaccaccacttcttctgggttccaatggttggtccattcatcggtgctttgatgggtggtttggtttacgacgtttgtatctaccaaggtcacgaatctccagttaactggtctttgccagtttacaaggaaatgatcatgagagcttggttcagaagaccaggttggaagaagagaaacagagctagaagaacttctgacttgtctgacttctcttacaacaacgacgacgacgaagaattcggtgaaagaatggctttgcaaaagactaagactaagtcttctatctctgacaacgaaaacgaagctggtgaaaagaaggttcaattcaagtctgttcaaagaggtaagagaactttcggtggtatcccaactatcttggaagaagaagactctatcgaaactgcttctttgggtgctactactactgactctatcggtttgtctgacacttcttctgaagactctcactacggtaacgctaagaaggttacttaa

SEQ ID NO: 33 - DNA, 1719 bp, Kluyveromyces marxianus
atgtctgaaaacactcaatacgacaacactagagactctggtggtcaatctccagttaacaacaacaacgcttggcaagaatctggtttcgctcacactagaccaagaagatacactactagatcttctgtttctgaaagacaatctggtttgtctggtttggaagaagaagactctgacatcgacgaccaaactactgttccagttactgcttacgttcaacaatacttggacgaaggttcttacttcccagttcaagaagttgttccaaacacttctttgaacatgaacatgtacagaagaaagagaggtaacactgttacttctaacgttatcgcttctagaccaatggaagctaactacactggttctgtttcttctccagacccagctttgcaaaaccaaaacaacgaaggtggtgttccagctaacgacccaaacgacccaaacaacgttaacaacgctatcactatgatggttaagccaaagactttgtaccaaaacccacaaactccaactgttttgccatctacttactacccaatcaacaagtggtcttctttcaagtaccaacacatgaaggaattcttcggtgaattcttgggtactatgatcatgatgatgttcggtactgctgttgtttgtcaatctaagttgtctgaacaagacaagatcaaccaattcaaccaaatcttggctatgaaccacaagtctaacgacgacatctctatgttgcaatacatcgctactccaaacgttgctggtaacttcgtttctatcgctttcggttgggctggtgctgttgttatgggttacttcgctgctggtggttctgctatctctggtgctcacttgaacccagctatcactgtttctaacttcatctacagaggtttcccattcagaaagttgggtttctacttcatgggtcaatacgttggttcttacttgggttctttgttgatgatctggtactaccacaaggttatcgctcacgtttacccaaactggccacaagaagaatctgttgttgctatgttctctgttgttccattggactacttgtctactccaagacaaatcatcgctgaattcgttatcggtgctatgttgcaatgtggtatcttctctttgactgacccatacacttgtttgtctactgacttgttcccagttatgttgttcatcttgatcttctctttgaacgcttctggtgcttaccaaactggtgctgttttgaacccagctagagacatgggtccaagattggctttgtggactatcggtatggacaaggacgttatcttcaactctcaccaccacttcttctgggttccaatggttgttccattcttgggttctttcgctggtggtttggtttacgacttctgtatctaccaaggtcacgaatctccattgaacttgccattgtctgcttacactgactggttcagaagacaatgggacactatcaagttgaagacttcttctggtttgaagggtactgacttggaaactatcgactctggtcacactttgtctcacatcgaatctcacagatctcaattgtctgaaaacaagcaagttcacttcaagtctgttttgagaaactctaagttgagaaacccatctactggtgttccaactatcttcgaatctgaagaaactacttactctagaccaaacttcgaacaaaagacttctaacggttctatctaa

SEQ ID NO: 34 - DNA, 1932 bp, Torulaspora delbrueckii
atgaacgacttcttggctgacccagaatctagaccagttgacatgaagaagtctactgacgacccacacttcactgaacaaagatctagatcttctactaaccactctcacaagtcttctaactctaacgacgaatacgactacgaaatgcaatcttactggccaagaccatctttctctagaggtcaatctagaacttcttacatcccacaatactctacttacaacggtggtcaattcccaatgcaccacgttgttccaaacactcaaatggctatgtcttcttctgctgacacttctggtggtgctaacggtcacttgccaggtactgaaaactctaagtctgaattgagaccaagagctactactgtttcttctaacttcgttaacttgggtgaattcttcagaaacaacgacgacatggaatctcaccacacttctgaccacagatctcacagaggttctacttctaacaagtctaacactgaccactcttactctaacgaagacaacgaatctgacccaagaaacgttccaatgatggttagaccaaagactttgtaccaaaacccacaaactccaactgttttgccatctacttaccacccaatcaacaagtggtctactgttaagcactcttacttgaaggaattcttggctgaattcatgggtactatgatcatggttggtttcggttctgctgtttgttgtcaagttttcgctgctggtaagatccaacaaaaccaattcgacgacgctttgtctttgttgactaacgcttctggtgaattggttgaaactgctaagactttcaagtacttggttacttctgttaacggtggtactttcgacgacgttgctttcggttgggctggtgctgttgttatgggttacttctgtgctggtggttctgctatctctggtgctcacttgaacccatctatcactgttgctaactacatcttcagaggtttcccatctaagaagatcccatactacatcgctggtcaattgactggtggtttcgttggtgctttgatcatcttcatcttctacaagaaggttttgcaagaagcttacactgaatggtggacttctgaatctgttgcttctatgttctgtgttttcccaaagccatacttgtcttctactagacaattcgtttctgaattcgtttgtactgctatgttgcaagcttctactttcgctttgactgacccatacacttgtttgtcttctgacatcttcccattgttgttgttcgttcaaatctacgttatcaacgcttctttgtcttaccaaactggttctgctatgaacatggctagagacttgggtccaagattggctttgtacgctgttggtttcgaaagatctgttttgtggtcttctcacaagcacttcttctgggttccaatcgttgctccattggttggttctatgactggtgctttggtttacgacgtttgtatctaccaaggtcacgaatctccagttaactggccattgtctgtttacaaggacatgatcttgagagcttggatcagaagaccaggttggaagaagagaaacagaggtagagctacttctgacttgtctgacttctcttacgacgacgacgacgacgacgacaacaacaacgaccaagacaacgactctcaaaacagattggctccaccaagaactagaactagatctactggttctgacactgaagaccaaccaagacaaaagggtgttcaattcaagtctatgcaaggtagagctaagagattctacggtggtgttccaactatcttggaagacgaagactctatcgctactgcttctttgggtggtgctgcttctgaatctgttgctttgtctgacgaatcttctactgaaggtaacaacgaagttgacgacggtgaacacgacagaaagactagataa

SEQ ID NO: 35 - DNA, 1959 bp, Candida glabrata
atgtctcaccaacaaggtggttctaagaacccaaacgctatgtctgacttgaacgaatacttgtctaacgaagacagaaactctcaagaaagaaacgacagagacgacttcgacgttgaaatgcaagaatacactccacaaccattcaagagaccaactgcttcttacatcccagaatacatcggtacttctaaccaattcccaatccaagaagttgttccaaacactaacatcccaatccaccaattgactgaaaaccactctgctgctcaatctccaaacagaccatcttctccagttaacaactctaacatgaacaacttgtctggtgacatgtctactgctgctgctacttctgttaacgttaacactacttctaacactgaccacttgagagctagagaccacactactgtttctgctaacgttttgaacttgggtcacttgtacaagaacaactacggtaacaacaacgacgacggtgaccacatctctgttcaagaaggtgttactaacgactctgaatctaagcaatacggtactagaagagacagagctacttctttcatctctagattggctggtggtggtgacgacggttctccaaacgacccaaacccaggtaacgcttctgttccaatcatcgttaagccaaagactttgtaccaaaacccacaaactccaactgttttgccatctacttaccacccaatcaacagatggtctttggttaagtctggtgttttgaaggaattcttggctgaattcatgggtactatggttatgatcatcttcggttctgctgttgttatccaagttttgtctggtggtaaggctcaacaagactcttacttggctgctatggacgctttgtctcaatctgacttgtctgctggtgaaaagatggctttcgaaaacttgactaagttggtttcttctgtttcttctggtactttcgacgacatcgctttgggttgggctgctgctgttgttatgggttacttctgtgctggtggttctgctatctctggtggtcacttgaacccaatcatcactttggctaacttcgtttacagaggtttcccagctaagaagatcccattctacttcttcggtcaattgttcggtgcttacgttggtggtttgatcgcttacggttactacaagaaggttatctctgaaactttcccagaccacttcaactctgaaactgttgtttctatgttctgtgttgttccaaagccatacttgtcttctgctagacaattcgtttctgaattcttgtgtggtgctatgttggttgcttgtactttcgctttgactgacccatacacttctttgtctggtgacgttttcccattgatgttgttcttgttgatcttcatgtgtaactctggtttgggttaccaaactggtactgctatgaacatggctagagacttgggtccaagaatggctttgtacactgttggtttctctagaaagttgttgtggacttctcaccaccacttcttctgggttccaatctgtgctccattcatcggtgctttgactggtggtttggtttacgacatcttcatctaccaaggtcacgaatctccagttaactggccattctctttgtacaaggaaactttccaaagatggtggttcaagagaccaggttggcaaagaagaaacaaggctagaagaatgtctgacttgtctgaaatctcttacgctgaagacgaagacttggacaacacttacactggtactagattcccaagagttactaagactaagtcttaccactcttctcacaacactgacgaaaagaaggttcaattcaagtctgttcaaagagacaagccacacaaccaaaacatggctgctgttttggacgacgaatcttctttggaaactgcttctttgggtgactcttacatcgaacaatactcttctaagaactctaactaa

SEQ ID NO: 36 - DNA, 2061 bp, Zygosaccharomyces parabailii
atgccaccaacttctcaacaagctatgaacgaattcttgtctaacccagacgctgctccaccagctcaaactggttctactgctgcttctggtgctggttctgaagctggtgctgacgctagagctggtgctgctgaaggtactgaatctgaagttccagcttctgctactggtccaatcccagttccatctgctacttctggtcaccacggttcttctatctctggtgctccatctgctagaggttctgactacgactacgaaatgcaagactacagaccaactccattcactactagaacttctagagctagatctaacactccatacatcccacactacatgatggctcaaggtcaccaattcccagttcaagaagttgttccaaactctcaaatggctatcgctactggtgttgacccaaacgcttctagatctcacatggactctatgagaggtaacatgagatctagaactcaaactgttacttctaacgttttgaacccaggtgaagctagaccatctagatctactactagaccaggttctcacgtttctgacggtgctggttctaagcacggttctgaagaagacgtttctcacgaagaccacggtgactctgaagaacacgctttgaacgttccaatgatggttaagccaaagactttgtaccaaaacccacaaactccaactgttttgccatctacttaccacccaatcaacaagtggtcttctttgaagcacggttacttgaaggaattcttggctgaattcatgggtactatggttttgatcgttttcggtactgctgttacttgtcaagttaacactgctgctaagatccaacaagacggtttcgaccaagctttggctcaattgactaacactccaggtggtttggttcaaactgctgaaactttcaaggaattggtttcttctacttctggtggtactttcgacgacgttgctttgggttgggctgctgcttctgttatgggttacttcgctgctggtggttctgctatctctggtgctcacttgaacccatctatgactgtttctaacttcatcttcagaggtttcccattcaagaagatcgttaactacatcgctggtcaattgttgggtgctttcgctggtgctttgatcttgtacatcttctacaagagagttatcgaagaagctttcccacacgaatggtggaagactgaatctgttgcttctatgttctgtgttttcccaaaggcttacttgtctactgctagacaattcgtttctgaatacatctgtactgctatgttgcaagttggtatcttcgctttgactgacccatacacttgtttgtcttctgaattgttcccattgatgttgttcatcttgatctacatcgttaacgcttctatgtcttaccaaactggttgtgctatgaacatggctagagacttgggtccaagattggctttgtacgctgttggtttcaacagacacttgttgtggatcaagcacaagcacttcttctgggttccaatcgttgctccattcttgggttctatcactggtggtttgatctacgacatctgtatctaccaaggtcacgaatctccagttaactggccattggctacttacagagacatcatctactctatgtggttgaagagaccagactggtctagaagaccatggagaagatctaacgaaaaggactctggttctgacttcaactctttctcttacgacgaagacgaagacgaaccagctccataccaacaagaaaacttgccaaagttgtctatgtctgactctggtaacccagaattgcaagaaagaccacaatctgttcaattcaagtctgttcaatctagaactaagagacacttcggtggtatcccaccaatcactgaagaagacccatctttggacggtgcttctttggctggtacttctatctctttggttggttctgctaacgacttcgacacttctactgctgctgctccaggttctcaaccaactgaagacttgttctctccaggtgctactcaaaagaagcaagaataa

SEQ ID NO: 37 - DNA, 2079 bp, Zygosaccharomyces rouxii
atgccagacactcaagacggtaagtctaactctcaaaagttgttgaacgactacttgtctaacccagacccaggtccatctccatctgctccacaaccaactactgctggtactggtgacacttctactactgctgctgacgttaacttgcaaggtaacaagaacttcgacaactctggtaactctggtatcgaatctagacacaagtcttcttctaactggaacgacgacggttacgactacgaaatgcaaaactactacccacaaccatacactcaaagaacttctagagctagatctaacactaactacatcccacactacatgactggtccagactctcaatacccaatccaagaagttgttccaaacactcaaatggctgttgctactggttctgacccagctgctaacagagctgacggtttcggtcactctgttagatctagagctccaactatcagatctaacgttccagacttccactctatcttgggttctactagatctaagccacactctcacaacggttctcacatcagatctaactcttctggtagatcttctgctgcttctgctgctgctgactctgaaaaccacggtaacgaaactggtgacggtgttaactctggttcttctaaccacgctttgtctactccattgatggttagaccaaagactttgcaccaaaacccacaaactccaactgttttgccatctgcttaccacccaatcaacaactggacttctttgaagcacggttacttgaaggaattcttggctgaattcgttggtactatggttatgatcatcttcggtaacgctgttaactgtcaagttaacgttgcttctaagatccaacaagaaaacttcgacaaggctttgcaaaaggtttctaactctccagaacaattgagagaaactgctgaagctttcaagaacttggtttcttctacttctggtggtactttcgacgaagttgctttgggttgggctgctgctactactatgggttacttcgctgctggtggttctgctatctctggtggtcacttgaacccatctatcactgttgttaacttcatcttcagaggtttcccattcaagaacgttttcatctacgttactggtcaattgttgggtgctttcgttggtgctttgatcttgttcatcttctacaagagagttatcgaagaaggtttcccaggtgaatggtggaagaacgaaactgttgctggtatcttctgtgttttcccaaagccatacttgtctacttctagacaattcgtttctgaatacatctgtactgctttgttgcaaatcggtactttcgctttgactgacccatacacttctttgtcttctgacttgttcccattgatgttgttcatcttgatgtacatcttgaacgcttctttgtcttaccaaactggttctgctatgaacatggctagagacttgggtccaagattggctttgtacgctgttggtttcaacagacacatgttgtgggttaaccaccaccacttcttctgggttccaatcgttgctccattcttgggttctatcactggtggtttgatctacgacgtttgtatctaccaaggtcacgaatctccagttaactggccaatcgctacttacaaggacatcttgagaagatcttggttgagaagaagacaatggaagggtagatctaactggccagttatcggtaagaagttgtctaacaacccagctccatctgaattctctgacttctcttacgaagacgacgacgaagacagaggtaacccattccaaaacccaaagactgacccatctaaggtttctttggttcaagaagctgacccaggtgctgacactagatctccagactctgttcacttcaagtctgttcaaggtgactctagaagattgcacggtgaaatcccaactatcatggaagaaaacccatctttggaaactgaatctttgggttctttgacttctttgtctatcaacaacaacgacaacaacggtccaacttctaacgacggtatcccatctttcccaactgttcaaaagaagcaagaataa

SEQ ID NO: 38 - DNA, 1692 bp, Kluyveromyces lactis
atgtctcaaactgctcaatacgaaccagttaaggacgctggtatctctaacggtgactggcaaaacgacgacttcgctaacgttaaccacagatacccaactggttctgttgacggtaacgaatctagaatctctggtgaaggtggttacgacgacgacaacgactctgctgacgacggtgctactgttccagttactgcttacgttcaacaatacttggacgaaggttcttacttcccagttcaagaagttgttccaaacacttctttgaacatgaacaactacagaagaatcagatctaacactgttacttctaacgttatgccaccaagaccaactgaaggtccaggttctgttatgtctagatctactactggtccaaaccaaaactctcaaactgctgctgacccaaacgacccatctaacgttaacggtgctgttactatgatggttaagccaaagactttgtaccaaaacccacaaactccaactgttttgccatctacttactacccaatcaacaagtggtcttctttcaagtaccaacacatgaaggaattcttcggtgaattcttgggtactatgatcatgatgatgttcggtactgctgttaactgtcaaagaaagttgtctcaacaaaaccaaatcaacaagttcaaccaaatcatccaattgaacaacatggaatctgaccaaatcgctatgttgcaatacttggctactccagacgttgctggtaacttcgctactgttgctttcggttgggctgctgctgttgttatgggttacttcgctgctggtggttctgctatctctggtgctcacttgaacccagctatcactgtttctaacttcgtttacagaggtttcccatggagaaagttgggtgtttacttcatgggtcaatacttgggttcttacatcggtactttgttgatcttgtggtactacagagaagttatcgaacacgtttacccaaactggcacttggaagaatctgttttggctatgttctctgttgttccattggactacttgtctacttctagacaaatcatcgctgaattcttgatcggtgctatgttgcaatgtggtatcttctctttgactgacccatacacttgtttgtctactgacttgttcccaatgatgttgttcatcttgatgttcatcttgaacgctgctggtgcttaccaaactggtgctgttttgaacccagctagagacatgggtccaagattggctttgttgactatcggtatggacaaggacgttatcttcaacactcaccaccacttcttctgggttccaatggttgttccattcgttggttctttcactggtggtttggtttacgacttctgtatctaccaaggtcacgaatctccattgaacttgccattgtctgcttacactgactggttcagaagacactgggaattgttgaaggttaagacttcttctggtttcgttggttctgacttggaaactatcggtactaacaacactacttctaacgttgaatctcacagatctcaaacttctgaaaacaagcaagttcacttcaagtctgttttgagaaactctaagactagaaacccatctactggtatcccaactatcttcgaatctgaagaaactacttactctagaccaaacttcatccaaaagcactctgacagatctgcttaa

SEQ ID NO: 39 - DNA, 1158 bp, Yarrowia lipolytica
atgctcaaccagccgaaaaaacccctgtggccgaaagtcagacacttcttgcgagaaccgtttgccgagttctggggctgcgtcattctcatcgttctgggagacggttctgttgcccaggtcactctctccaacggcgagaagggagactaccagtccatttcgtggggctggggtctgggagtaatgtttggcgtctacgtcagcggaggtatctctggaggccatctcaaccccgccgtcacgcttgcttcttgcgtctacagaggcttcccatggagaaaattccccggatacatgctggcccagaccctaggatgtatggtaggtgctgccatcatctacggaaactaccgatctgcaatcgatacgtttgagggctgcaagggctgtcgaactgtgtctggtcccaaatccacagccggagtattctgtacctaccctgctcccttcatgactcgaactggccagttcttttccgaaattgtagcctcggctgttctgcagtttatcattttcgctattaacgacaccaaaaacatacccgctggtcctctggctcccctggtactcttcttcctcattttcgctattggagcctgtcttggatgggagactggatatgccattaactttgcacgagactttggtccccgacttgtgactgccatgatcggatacggcagcgaagtctggagcgctggaggttactacttttgggtccccatcgttgcccccttcattgggtgtctgctgggcggctttctctacgactttttcatgtacaccggggacgaatcgcccatcaactggccctggatgggattcgaccgatttctcaacccccacaagcgaatcgaacatgatatgggaactgttcagcagaacgtggaggctcccatgcttgtggaagcccaccccaacatgggctcagtgcaggagaaccctctgtcgacgggaaccgacgaacccaaggtcgatatggaccctggcttctcgtcagatagccagactgtgcatctgggcagaaatatgcgagccgctgaccatgaacatgtcgagcaggctcacacgcctgagtcggccactcctccccagcctacaggcgccgcccagtttctggagttcgaaaacctcgacgactcggacagtagttag

SEQ ID NO: 40 - DNA, 969 bp, Yarrowia lipolytica
atgacagacgcaattgtacactcgcccgaaacccccctgtggccgcgaatccgacaccagctccgagaaccgtttgccgagttctggggatgtctgattctcattctgctgggagacggagtggtggcccaggtgaccctctccggcggcaaaaacggagactaccagtccatttcctggggctggggtctcggagtcatgtttggcgtctacgccgctggtggaatttccggcggccatctgaaccccgccgtgactctctgctcctgtatctaccgaggtttcccctggcgcaagtttcccatctatctggtggcccagttgctgggatgtatgaccggagctgctctggtctatggaaatcaccggtctgccattgacgtttttgagggcggcaagggcattcgaaccgtcggattgcccacttctaccgccggaatcttctgcacctaccccgccgagtttatgagcaccaccggccagtttttctccgaggtcattgcctcagccgtgctccagtttgccattttcgccatcaacgaccaaaagaacctcgccgccggccccctggcacccctgatcctctttttcctcattttcgccatcggcgcatgcctcggatgggaaaccggatatgccattaacctggcccgtgatttcggcccccgattggtcaccgcaatgatcggctacggctccaaggtctggaccacgggcaactactacttctgggtgcccatcatcgcgcccttcatcggcgctgccctcggcggctttttctacgacctgttcctctacaccggcgacgagtcgcccctcaactggccctacatgggattcgaccgaattttctacctgcttggaaagaaggaggctcccagaatcgaacatgatatgggtatggttgaggaggctcccagtaaggaggaaattgcccatttttccaattcccctaatgtttctagttag

SEQ ID NO: 41 - DNA, 1395 bp, Azotobacter vinelandii
atggctgtttacaactacgacgttgttgttatcggtactggtccagctggtgaaggtgctgctatgaacgctgttaaggctggtagaaaggttgctgttgttgacgacagaccacaagttggtggtaactgtactcacttgggtactatcccatctaaggctttgagacactctgttagacaaatcatgcaatacaacaacaacccattgttcagacaaatcggtgaaccaagatggttctctttcgctgacgttttgaagtctgctgaacaagttatcgctaagcaagtttcttctagaactggttactacgctagaaacagaatcgacactttcttcggtactgcttctttctgtgacgaacacactatcgaagttgttcacttgaacggtatggttgaaactttggttgctaagcaattcgttatcgctactggttctagaccatacagaccagctgacgttgacttcactcacccaagaatctacgactctgacactatcttgtctttgggtcacactccaagaagattgatcatctacggtgctggtgttatcggttgtgaatacgcttctatcttctctggtttgggtgttttggttgacttgatcgacaacagagaccaattgttgtctttcttggacgacgaaatctctgactctttgtcttaccacttgagaaacaacaacgttttgatcagacacaacgaagaatacgaaagagttgaaggtttggacaacggtgttatcttgcacttgaagtctggtaagaagatcaaggctgacgctttcttgtggtctaacggtagaactggtaacactgacaagttgggtttggaaaacatcggtttgaaggctaacggtagaggtcaaatccaagttgacgaacactacagaactgaagtttctaacatctacgctgctggtgacgttatcggttggccatctttggcttctgctgcttacgaccaaggtagatctgctgctggttctatcactgaaaacgactcttggagattcgttgacgacgttccaactggtatctacactatcccagaaatctcttctgttggtaagactgaaagagaattgactcaagctaaggttccatacgaagttggtaaggctttcttcaagggtatggctagagctcaaatcgctgttgaaaaggctggtatgttgaagatcttgttccacagagaaactttggaaatcttgggtgttcactgtttcggttaccaagcttctgaaatcgttcacatcggtcaagctatcatgaaccaaaagggtgaagctaacactttgaagtacttcatcaacactactttcaactacccaactatggctgaagcttacagagttgctgcttacgacggtttgaacagattgttctaa

SEQ ID NO: 42 - DNA, 1400 bp, Escherichia coli str. K-12
atgccacactcttacgactacgacgctatcgttatcggttctggtccaggtggtgaaggtgctgctatgggtttggttaagcaaggtgctagagttgctgttatcgaaagataccaaaacgttggtggtggttgtactcactggggtactatcccatctaaggctttgagacacgctgtttctagaatcatcgaattcaaccaaaacccattgtactctgaccactctagattgttgagatcttctttcgctgacatcttgaaccacgctgacaacgttatcaaccaacaaactagaatgagacaaggtttctacgaaagaaaccactgtgaaatcttgcaaggtaacgctagattcgttgacgaacacactttggctttggactgtccagacggttctgttgaaactttgactgctgaaaagttcgttatcgcttgtggttctagaccataccacccaactgacgttgacttcactcacccaagaatctacgactctgactctatcttgtctatgcaccacgaaccaagacacgttttgatctacggtgctggtgttatcggttgtgaatacgcttctatcttcagaggtatggacgttaaggttgacttgatcaacactagagacagattgttggctttcttggaccaagaaatgtctgactctttgtcttaccacttctggaactctggtgttgttatcagacacaacgaagaatacgaaaagatcgaaggttgtgacgacggtgttatcatgcacttgaagtctggtaagaagttgaaggctgactgtttgttgtacgctaacggtagaactggtaacactgactctttggctttgcaaaacatcggtttggaaactgactctagaggtcaattgaaggttaactctatgtaccaaactgctcaaccacacgtttacgctgttggtgacgttatcggttacccatctttggcttctgctgcttacgaccaaggtagaatcgctgctcaagctttggttaagggtgaagctactgctcacttgatcgaagacatcccaactggtatctacactatcccagaaatctcttctgttggtaagactgaacaacaattgactgctatgaaggttccatacgaagttggtagagctcaattcaagcacttggctagagctcaaatcgttggtatgaacgttggtactttgaagatcttgttccacagagaaactaaggaaatcttgggtatccactgtttcggtgaaagagctgctgaaatcatccacatcggtcaagctatcatggaacaaaagggtggtggtaacactatcgaatacttcgttaacactactttcaactacccaactatggctgaagcttacagagttgctgctttgaacggtttgaacagattgttcta

SEQ ID NO: 43 - DNA, 654 bp, Aspergillus oryzae
atgtctgtttctttgactttgagatctgctttgggtccatgtagagctgcttctatcgctagaccaggtaagtctttgttcgctttccaacactctgttatccaatctagaactccatacgttccataccaaagaaacgcttacgaaagaccatctgtttctagatggccacaagttgctagaagatctgcttcttcttcttcttctccatctccagttccagttgttccatactcttctttgactgttggtgttccaagagaaacttacccaaacgaaagaagagttgctatcactccacaaaacgttgctttgttgttgagaaagggtttctctagagttttgatcgaaagaggtgctggtgaagctgctgaattgttggaccaagcttacgaacaagctggtgctactttggttgacagagctactgtttggtctcaatctaacatcatcttgaaggttagaggtccacaaccaggtgacgaaatcgaagctttgcaacaaggttctactatcatctctttcttgtacccagctcaaaacaagcaattggttgaccaattggcttctagaagagttactgctttcgctatggacatggttccaagaatctctagagctcaaactttcgacgctttgagatgtgttgcttaa

SEQ ID NO: 44 - DNA, 1422 bp, Gluconobacter oxydans
atgatcttgttggcttacatcgttgctttgtctggtttgatcgcttctggtttgttggtttacggtttgaagagaatgtcttctccagttactgctgtttctggtatcgttactgctggttggggtatgttgttcgttgttgctgcttctttcttgcaaatcttctctgtttctgacgctgctcaaccacacatcgttgttaacgttatcttggctgttttggctttggttatcggtggtggttgggctggttggaagggtagatctgttgctatgactgctatgccacaaatggttgctttgttcaacggtatgggtggtggtgctgctgctgctgtttctgttatcgctttgactggtccaaaggacactggtgttggtcaattgttggttactgttgctggtggtttgatcggttctatgtctttgtctggttctttgatcgcttgggctaagttggacggtagaatgaacaagccaatcagattcggtggtcaaagaatcgttaacgctgctgttttcatgttgactatcttgttgggtgttatggctgttgctcaataccaccaaccaggtggtggtttgttcgctttgttgttcttcatcggtgctttgttgtgtggtatctgtatgactgttccaatcggtggtgctgacatgccagttgttatctctttgtacaacgctttcactggtttggctgttggtttggaaggttacgttatggctgacccagctttgatgatcgctggtatggttgttggttctgctggtactttgttgactgttatgatggctaagggtatgaacagatctatcactaacgttttgttctctaacttcggtgaagctactgctgctactgcttctggtccacaaaaggaagctaagtctgtttctgcttctgacgctgctactactatgagatacgcttctactgttatcatcgttccaggttacggtttggctgttgctcaagctcaaggtaagttgtacgaattcgttaagttgttgcaagctgctggtgttgacgttaagttcgctatccacccagttgctggtagaatgccaggtcacatgaacgttttgttggctgaagctggtgttccatacgacatgatctacgacatggacgacatcaacgactctttcgctgacactgacgttgctttggttatcggtgctaacgacgttgttaacccatctgctagaactgacaagtcttctccaatctacggtatgccaatcttgaacgctgacaaggctagacaagttttcgttatcaagagaggtatgggtatgggttactctgctgttcaaaacccattgttcttccaagacaactgtgctatggttttcggtgacgctcaagctgttttgtctaagatggttgaagctgttaagggtttgtctgcttcttaa

SEQ ID NO: 45 - DNA, 1425 bp, Bifidobacterium breve
atgacttctgctactgttggtgctatcgacatcgttgcttggttcgtttacttgttctctgctgttttgttcgttgttggtttgcacttcatgaactctccaaagactgctagaaagggtaaccaaatctctgctttcggtatggttgttgctgttttgatggctttcatcgttttgttcgctaagggtttcgttaacgttgttgctgttgttgttttggttgttggtatcttgatcggtgctgttgctggtgttgtttctgctaagaaggttaagatgactgacatgccacaattggtttctgttttcaacactgttggtggtggtgctgctgctttggttgctttgaacgacatcttgactaaggaaggtactccagacatcgttgttttgatcactgctggtttgggtatcttgatcggttctgttactttcactggttctttgatcgctgctggtaagttgcaaggtatcaagtgggttaagaagttgactatgccaggtaagggtgtttggaacatcttgttcatcgttttgactatcgcttctttcgttatgttgtgtgttcaaccagaaagaagattgttgtggtctatcttgactactgttttcgctttgtgttacggtttggttttcgttatcccaatcggtggtgctgacatgccagttgttatctctgttttgaacgcttgtactggtactgctgttgctatgtctggtttggctatcgacaacgttgctttgatcgttgctggtgctttggttggttctgctggtgttactttgtctatcttgatggctcaagctatgaacagaccattgttgtctgttttggctggtggtttcggtggtggttctgacgctgctgctgctggtgacggtccagaaggtactatgaaggaaactactgctgacgacttggctgttcaattggtttacgctcaaaaggttatcttcgttccaggtttcggtttggctcaagctcaagctcaaagagaattggctgacttgggtgaattgttgaagggtcacggtgttgaagtttcttacgctatccacccagttgctggtagaatgccaggtcacatgaacgttttgttggctgaagctaacgttccatacgaagaattggttgacttggacgaaatcaacccacaattcccacaagctaacgttgctttggttgttggtgctaacgacgttactaacccagctgctagaagaccaggtactccagtttctggtatgccaatcttggacgttgacaagtctcaaaacgttgttgttatgaagagaggtagaggtatgggttacgctggtatccaaaacgaattgtacttcgaaggtaacactcaaatgttgttcggtgacgctaaggcttctttgcaagctgttatcgctgctgttaaggaattgatctcttaa

SEQ ID NO: 46 - DNA, 25 bp, Artificial Sequence
  Synthetic
tgaataggagacttgacagtctggc

SEQ ID NO: 47 - DNA, 25 bp, Artificial Sequence
  Synthetic
ctctgagatcatccgagcattcaag

SEQ ID NO: 48 - DNA, 26 bp, Artificial Sequence
  Synthetic
atgccccctttcaccctggcagacac

SEQ ID NO: 49 - DNA, 26 bp, Artificial Sequence
  Synthetic
ctataacccggcacagagccttggcg

SEQ ID NO: 50 - DNA, 3707 bp, Artificial Sequence
  Synthetic
agccaccaacacggacgatttcctggttcgaatgatcaagggatgcatccagctgggtgagattcccaacatccacaactcggtcaacatggtgcccgtcgatcacgtggctcgggttgttactgccgcctctttctggcccaagcagccttccggcgtggttgtcgcccatgtgacttcccagcctcgaacacggttcaacgaattcctgcagaccctccagaagtacggttacaaagtttctgtcgaggactacgtcacctggcgtctggctctggagaagtttgttgttgaggactcgcaggactctgccctgtatcctctgctacactttgtgctcgatgaccttccccagtctaccaaggcccccgagctggatgactccaatgctcgatctgctctctctcgagacgctgagtggaccggagtcgatttgtccgctggtaagggcgttgacgaggctcaaatgggtatctacctggcttacctggtggctgtcggcttcctggatgccccccagtccaaggttgagcttgctttgccgaaggtggagctgtctgagcagacccttgataagctgaagagtgtcggtggacgtggtggtaacaagtaagcagtgccgtagggagtgccttgaccataaggcgatgcgaagcattgccattttgttatttgttaccgtgtaatggtgattattgcttgtctgtgagcagactatttttgtatgatttaattaattatgatatatatgactaaatgtgaggtgtcgcaataattacagcatttttcgtttgagatggtttgtattgtagccagtgctcaaaaattgagcgtaaatttgatagcgtttgctgatgagcaagtggaagcatgggaatctcatccccagaactcgtaatagttacatacggcaatacaactatcagtgacatcacatacatgccagttgtcacgcaaggttctacaaactcatggggttccatttaaatatactctatatcaatacttatatcagacgggataacttcgtataatgtatgctatacgaagttatatgcttttgcaagctttccttttccttttggctggttttgcagccaaaatatctgcatcaatgacaaacgaaactagcgatagacctttggtccacttcacacccaacaagggctggatgaatgacccaaatgggttgtggtacgatgaaaaagatgccaaatggcatctgtactttcaatacaacccaaatgacaccgtatggggtacgccattgttttggggccatgctacttccgatgatttgactcattgggaagatgaacccattgctatcgctcccaagcgtaacgattcaggtgctttctctggctccatggtggttgattacaacaacacgagtgggtttttcaatgatactattgatccaagacaaagatgcgttgcgatttggacttataacactcctgaaagtgaagagcaatacattagctattctcttgatggtggttacacttttactgaataccaaaagaaccctgttttagctgccaactccactcaattcagagatccaaaggtgttctggtatgaaccttctcaaaaatggattatgacggctgccaaatcacaagactacaaaattgaaatttactcctctgatgacttgaagtcctggaagctagaatctgcatttgctaatgaaggtttcttaggctaccaatatgaatgtccaggtttgattgaagtcccaactgagcaagatccttccaaatcctattgggtcatgtttatttctatcaatccaggtgcacctgctggcggttccttcaaccaatattttgttggatccttcaatggtactcattttgaagcgtttgacaatcaatctagagtggtagattttggtaaggactactatgccttgcaaactttcttcaacacagacccaacgtacggttcagcattaggtattgcctgggcttcaaactgggagtacagtgcctttgtcccaactaacccatggagatcatccatgtctttggtccgcaagttttctttgaacactgaatatcaagctaatccagagactgaattgatcaatttgaaagccgaaccaatattgaacattagtaatgctggtccctggtctcgttttgctactaacacaactctaactaaggccaattcttacaatgtcgatttgagcaactcgactggtaccctagagtttgagttggtttacgctgttaacaccacacaaaccatatccaaatccgtctttcccgacttatcactttggttcaagggtttagaagatcctgaagaatatttaagaatgggttttgaagccagtgcttcttccttctttttggaccgtggtaactctaaggtcaagtttgtcaaggagaacccatatttcacaaacagaatgtctgtcaacaaccaaccattcaagtctgagaacgacctaagttactataaagtgtacggcctactggatcaaaacatcttggaattgtacttcaacgatggagatgtggtttctacaaatacctacttcatgaccaccggtaacgctctaggatctgtgaacatgaccactggtgtcgataatttgttctacattgacaagttccaagtaagggaagtaaaatagataacttcgtataatgtatgctatacgaagttattagcgatattcaaaaatcgagacatatcatcacatgctatgactcagcccttggcgtcaaaaagcatgaattgcactgcagtgtgcagattcatgcaggacaaacaccagaccagtcaatgtaattggctgtggtgcacgtccaaatcaatggcacaattgcccgtgatgcatgctccttaggagacgctgtggggtttgtgcataggttacagcttccgggtcatgatcaacttccgcgcaacaattgtgtgctaaatcagggcagaagcaattgctcaattgatcacgggtagccaccgcaacattcgcactccttctcagttttcatactcacagccaaggttcggatggtgttgatggcagtctcgtcagtctttgaaaattggggagccattttgaaagttgtccgtctagcaagctaccagcagggtatatatagcggcagacccaaagttttcctagctcttccattcggttcatccatactttgtttcccagggtaactttcccaatccagtaactttgaggcttatacgactcaagcttttcgcaacctcagaccagagagagctacaacaaggagggtggtgacattttggaaggcgtgggaggtggtatcggggcggggagtgattgtagcgtgctcaattgggttgttaagggcttttagtcagccatataactgccttctaccatctattcgccctcttcagctattgctggatactaaggttgctgatgccttatcgtgtcagtcagcgacatcggacaacttccaccatatatgaaactacggacggatacacagtcagcgggtagtcatattcggaggggtcttcggagttcccaattggggttccgttggaagtcatctttgtgtggccaccgttttttcccgtcggaacaccatcttgaaaactccgccacttacacccgtcctacccactctccctcgcactactgtagctacgtacttttcctacttctcagacaccgtattccctcactcacccacctt

SEQ ID NO: 51 - DNA, 3767 bp, Artificial Sequence
  Synthetic
agaaacaaagggggggagttcatgatgtgctattgcaagaagaaaaacatataggatatttgtgtggaaaccccgcgcaaaaagcactccgatccacttcccagccacgtctcaaccctacacccccttttcgtcgaccatcgccgtcccctcctgcacagatcttcacctcccgggttattacagatccacactcctaaatttatgaaacacgccgatcagcgggaagagcccccgcccccaccatccattttgggcgtgcgctggagacctccaacgaggagggcacaaagacattgtggacaatgctcgtttaggcccttatacgagaaagtcgggggactagtgagcgatatacggcgaataggcgttttcccagatgggtgaagcgtagggttgtattacaggggtgggttaaggttcttcgaccgggagaggtcgtatggcctagatgagcatatgggtcgattcgtttcgccgcggagaactcgatgccgttttcagatttccattggcatctatagttgtcgagatatcccctctgaaaaagccgttgctatttagaccctgtttttcgtgcgtcagctatgcggtccaatttgaggcagtcagggtaagtgtttcaagtgggcgttggaaaatgacgaagtgaccgtctatagccgcggcgagagctgaaataaaccgccctgagtgatttatcacgtgatctgacaccagcggcgttcttcccccattcccatagggttcttagcgcatctcgaatattgtgcattcctctgtccaccaaattagctcatgcggggaaaaagcttactcgtaccttcttataatacaccctacatgtccataaagtggtggcactctaccaacatcacctgttataatgccccctttcaccctggcagacactacggcgatacaggtgctcatgtcggcgacacaggatacccgctcggaataacttcgtataatgtatgctatacgaagttatatgcttttgcaagctttccttttccttttggctggttttgcagccaaaatatctgcatcaatgacaaacgaaactagcgatagacctttggtccacttcacacccaacaagggctggatgaatgacccaaatgggttgtggtacgatgaaaaagatgccaaatggcatctgtactttcaatacaacccaaatgacaccgtatggggtacgccattgttttggggccatgctacttccgatgatttgactcattgggaagatgaacccattgctatcgctcccaagcgtaacgattcaggtgctttctctggctccatggtggttgattacaacaacacgagtgggtttttcaatgatactattgatccaagacaaagatgcgttgcgatttggacttataacactcctgaaagtgaagagcaatacattagctattctcttgatggtggttacacttttactgaataccaaaagaaccctgttttagctgccaactccactcaattcagagatccaaaggtgttctggtatgaaccttctcaaaaatggattatgacggctgccaaatcacaagactacaaaattgaaatttactcctctgatgacttgaagtcctggaagctagaatctgcatttgctaatgaaggtttcttaggctaccaatatgaatgtccaggtttgattgaagtcccaactgagcaagatccttccaaatcctattgggtcatgtttatttctatcaatccaggtgcacctgctggcggttccttcaaccaatattttgttggatccttcaatggtactcattttgaagcgtttgacaatcaatctagagtggtagattttggtaaggactactatgccttgcaaactttcttcaacacagacccaacgtacggttcagcattaggtattgcctgggcttcaaactgggagtacagtgcctttgtcccaactaacccatggagatcatccatgtctttggtccgcaagttttctttgaacactgaatatcaagctaatccagagactgaattgatcaatttgaaagccgaaccaatattgaacattagtaatgctggtccctggtctcgttttgctactaacacaactctaactaaggccaattcttacaatgtcgatttgagcaactcgactggtaccctagagtttgagttggtttacgctgttaacaccacacaaaccatatccaaatccgtctttcccgacttatcactttggttcaagggtttagaagatcctgaagaatatttaagaatgggttttgaagccagtgcttcttccttctttttggaccgtggtaactctaaggtcaagtttgtcaaggagaacccatatttcacaaacagaatgtctgtcaacaaccaaccattcaagtctgagaacgacctaagttactataaagtgtacggcctactggatcaaaacatcttggaattgtacttcaacgatggagatgtggtttctacaaatacctacttcatgaccaccggtaacgctctaggatctgtgaacatgaccactggtgtcgataatttgttctacattgacaagttccaagtaagggaagtaaaatagataacttcgtataatgtatgctatacgaagttatgacggtgcctctggagaggagtagtcggagtactcctagtaggacgccaaggctctgtgccgggttatagacaaccctgaaatgttaccaacattgaggaagtgtctgaagcaggctgaagcatcgtagaagccttgtcatggtcacgtcagacccagggaagactctggaagtgctcatgtctgttctagacgttctttttgcactttcgcaagacgtatccgcggagtggtgactaaaactaagaggaattcttaaaagctgggctttcgcggattttggcaatttcggtggacccagagatctgacgttgccaaggagacggttcagacggcctcagacctgcacgagtccccagagtctcatctctacccccatgatgcggtttcccacagtcgtttcaacaatcattcggctagcagcaacctattctcttcttcaaagggctattccagaccagcaaatcttcgcctttttggccgagtctcctcgttgacgaacctgctctggaaacaaccccttccaatccttctgtactctacttttactaaccggctctcctgagcccgagctttcttagccagctccaaaacgcttgcctgtcgctggtagacgggtgattcatagtactaatcaatgcatttaatcagagtgagtggtatggagaacgtcgcccgagaagctgcagtagggacatcagaagggacatatctgcctgaagacaccctcgcgttaatcatgagtgcatatctcgtgatcagacacacgatatactgcgcggcagtacttgcatcgtcaagtggttgatatcaagggtatggcaaggttccgggtatcttgggtgtgaactacaagtagttggtaccgtatgtactactgacttgtaccagatatgaaggcacattacggaaacccgactcattatctgtctcgatagcgacaatgtcagctgggtgaacttgtctaccaatccaacccattgaatttttcaacattccacctcgtctggttcagtcgccatgcacctcaacctcaccacctgaaatcatcacgagatgatggacttttaagaagcccatttcactggtgcaagagaagctggtgataaatggcagttccctctgtatctgctctcaatgg

SEQ ID NO: 52 - DNA, 27 bp, Artificial Sequence
  Synthetic
ctatctccacaacaatgcctgcaccag

SEQ ID NO: 53 - DNA, 25 bp, Artificial Sequence
  Synthetic
ccggttacacatgactgtaggaaac

SEQ ID NO: 54 - DNA, 23 bp, Artificial Sequence
  Synthetic
ccatacacagcaccacctcaatc

SEQ ID NO: 55 - DNA, 23 bp, Artificial Sequence
  Synthetic
tctatatacatcctctaaggagc

SEQ ID NO: 56 - DNA, 3770 bp, Artificial Sequence
  Synthetic
gaggggggagatgccccatctctggcaaccctatttgacgatagttgctggaggcttgacaggacttggtgacgaggggtgtttgggcgctggaagcgtaattttcgtcttgaatgggccgtcgagacttggggttcgaccccgactaaatggcgcaccgctagattctcttttggcgactttctcgggattctagtcacccccgcaatgttccagcttacggtttgagacagtacacgactggctaggcgagttgttgaagtcgtagcgtagagtgggaggcatgacgtcacgggacagctgcgtgcaccacgcgagcaggtcaattgacctcatttgagtggtgtggcttggcgttctagcggtggcggcgttgtcgagctccctctacttgtagtgagattatgtcgacgagcgggggggacttccattgtgcttgccactgctagtgcagtacaactgaaagctaaaccgcaatcaatcccaaactgcatgtccgccttaactctgatatgttatcaagagagtggtgtggtgaggtgaggtgaggtgacgtggacaagttgatggggagttggggcattgacaaaagggaaattgcagggggattccgccggctatatatatcttatgtctgctcaattcccagacggctccacacaaaaccaagataccacaccatcatggtcacaccgggtacataactcccatccatctcatcccacttgcatggcgaccggagagagaaagcccggggagagcacgtcggcgcggtccccagggcgacaaccaaaacaaaatcaccgagtgactccgaaagccgcgttccaacacccccccaaaatccccccctcaaacacgtcagccacctgtcccccgaaaattaacttcactgacatggcgcagctattaaggctaaagtgaatgcatggctcatctttgtttgctggttgctactgtgactgaggtaaaaaccctcgctcccaagtctatatatacctgggtgtgctccctcgaacagacccgtcacagtaaaactactacctccatacacagcaccacctcaatcataacttcgtataatgtatgctatacgaagttatatgcttttgcaagctttccttttccttttggctggttttgcagccaaaatatctgcatcaatgacaaacgaaactagcgatagacctttggtccacttcacacccaacaagggctggatgaatgacccaaatgggttgtggtacgatgaaaaagatgccaaatggcatctgtactttcaatacaacccaaatgacaccgtatggggtacgccattgttttggggccatgctacttccgatgatttgactcattgggaagatgaacccattgctatcgctcccaagcgtaacgattcaggtgctttctctggctccatggtggttgattacaacaacacgagtgggtttttcaatgatactattgatccaagacaaagatgcgttgcgatttggacttataacactcctgaaagtgaagagcaatacattagctattctcttgatggtggttacacttttactgaataccaaaagaaccctgttttagctgccaactccactcaattcagagatccaaaggtgttctggtatgaaccttctcaaaaatggattatgacggctgccaaatcacaagactacaaaattgaaatttactcctctgatgacttgaagtcctggaagctagaatctgcatttgctaatgaaggtttcttaggctaccaatatgaatgtccaggtttgattgaagtcccaactgagcaagatccttccaaatcctattgggtcatgtttatttctatcaatccaggtgcacctgctggcggttccttcaaccaatattttgttggatccttcaatggtactcattttgaagcgtttgacaatcaatctagagtggtagattttggtaaggactactatgccttgcaaactttcttcaacacagacccaacgtacggttcagcattaggtattgcctgggcttcaaactgggagtacagtgcctttgtcccaactaacccatggagatcatccatgtctttggtccgcaagttttctttgaacactgaatatcaagctaatccagagactgaattgatcaatttgaaagccgaaccaatattgaacattagtaatgctggtccctggtctcgttttgctactaacacaactctaactaaggccaattcttacaatgtcgatttgagcaactcgactggtaccctagagtttgagttggtttacgctgttaacaccacacaaaccatatccaaatccgtctttcccgacttatcactttggttcaagggtttagaagatcctgaagaatatttaagaatgggttttgaagccagtgcttcttccttctttttggaccgtggtaactctaaggtcaagtttgtcaaggagaacccatatttcacaaacagaatgtctgtcaacaaccaaccattcaagtctgagaacgacctaagttactataaagtgtacggcctactggatcaaaacatcttggaattgtacttcaacgatggagatgtggtttctacaaatacctacttcatgaccaccggtaacgctctaggatctgtgaacatgaccactggtgtcgataatttgttctacattgacaagttccaagtaagggaagtaaaatagataacttcgtataatgtatgctatacgaagttataggatgtatatagataatgattgtttatgattagacattgattgagtgtagttggacattagcagtcagataggcaacgaagatcatccaagtctgaatacatacccatacaaatcatacaagtaaatgatggaattactcatataagtatgtacttacttgtaccgaattgccaatgaatgtcaatcagaacgcagtatgtacaagtactcgcacaatatcataaggcactcgaatgttcaagaagtcatcattttggtgattcggggaaatacttgacacctttgttgatgcaacttgactccataagtaggaaacccatagtatatctttttgtcgctttatattcacctgttcccttctttctatggactataagttaatttagttgaccttgtcaagtaatacctcaacaaactgtaaagaaataggagattattgcttttggtttttgagaagagatctggatagcaccacacaaataatgcgtcaaaatcagttcaaaatccaacccacagaacaaccaacactccccgaaccgctcataacctgtataggatgctgctccactcacacctcttgtccagccctaacctgcgatctaggttggggaaattttgtatgcaggaaaaatatatgcaagatttgggattttatttgtgttcatcaaccacgtcgatttacacagctatcatggtccgagaacgatctggaacggtggcgtacacgcccaaaaagcagcagaagcctctggtggacgacgcccacttgtcctctgctgaggaggacgatttcaagaccccagagagtgcaaagagcaagaagcctgaagcgtcacaacaggagccggcggtgtcagaaacgccagtcaagggcaaggccaagaagatcacctttgacgaggacggaatgagtgccgagcccatcgttgagaagaaaaaggtggttgtggaggagtctgaagacgacagtgacgatgcccccgaggaggaaacactggaggatggacaggaaaagaccctggccaagcaaaa

SEQ ID NO: 57 - DNA, 3830 bp, Artificial Sequence
  Synthetic
tataattttcgcaccaaaaagagaccgtttatcgtggattatgggggtgtgatgtggggggaggggggagatgccccatctctggcaaccctatttgacgatagttgctggaggcttgacaggacttggtgacgaggggtgtttgggcgctggaagcgtaattttcgtcttgaatgggccgtcgagacttggggttcgaccccgactaaatggcgcaccgctagattctcttttggcgactttctcgggattctagtcacccccgcaatgttccagcttacggtttgagacagtacacgactggctaggcgagttgttgaagtcgtagcgtagagtgggaggcatgacgtcacgggacagctgcgtgcaccacgcgagcaggtcaattgacctcatttgagtggtgtggcttggcgttctagcggtggcggcgttgtcgagctccctctacttgtagtgagattatgtcgacgagcgggggggacttccattgtgcttgccactgctagtgcagtacaactgaaagctaaaccgcaatcaatcccaaactgcatgtccgccttaactctgatatgttatcaagagagtggtgtggtgaggtgaggtgaggtgacgtggacaagttgatggggagttggggcattgacaaaagggaaattgcagggggattccgccggctatatatatcttatgtctgctcaattcccagacggctccacacaaaaccaagataccacaccatcatggtcacaccgggtacataactcccatccatctcatcccacttgcatggcgaccggagagagaaagcccggggagagcacgtcggcgcggtccccagggcgacaaccaaaacaaaatcaccgagtgactccgaaagccgcgttccaacacccccccaaaatccccccctcaaacacgtcagccacctgtcccccgaaaattaacttcactgacatggcgcagctattaaggctaaagtgaatgcatggctcatctttgtttgctggttgctactgtgactgaggtaaaaaccctcgctcccaagtctatatatacctgggtgtgctccctcgaacagacccgtcacagtaaaactactacctccatacacagcaccacctcaatcataacttcgtataatgtatgctatacgaagttatatgcttttgcaagctttccttttccttttggctggttttgcagccaaaatatctgcatcaatgacaaacgaaactagcgatagacctttggtccacttcacacccaacaagggctggatgaatgacccaaatgggttgtggtacgatgaaaaagatgccaaatggcatctgtactttcaatacaacccaaatgacaccgtatggggtacgccattgttttggggccatgctacttccgatgatttgactcattgggaagatgaacccattgctatcgctcccaagcgtaacgattcaggtgctttctctggctccatggtggttgattacaacaacacgagtgggtttttcaatgatactattgatccaagacaaagatgcgttgcgatttggacttataacactcctgaaagtgaagagcaatacattagctattctcttgatggtggttacacttttactgaataccaaaagaaccctgttttagctgccaactccactcaattcagagatccaaaggtgttctggtatgaaccttctcaaaaatggattatgacggctgccaaatcacaagactacaaaattgaaatttactcctctgatgacttgaagtcctggaagctagaatctgcatttgctaatgaaggtttcttaggctaccaatatgaatgtccaggtttgattgaagtcccaactgagcaagatccttccaaatcctattgggtcatgtttatttctatcaatccaggtgcacctgctggcggttccttcaaccaatattttgttggatccttcaatggtactcattttgaagcgtttgacaatcaatctagagtggtagattttggtaaggactactatgccttgcaaactttcttcaacacagacccaacgtacggttcagcattaggtattgcctgggcttcaaactgggagtacagtgcctttgtcccaactaacccatggagatcatccatgtctttggtccgcaagttttctttgaacactgaatatcaagctaatccagagactgaattgatcaatttgaaagccgaaccaatattgaacattagtaatgctggtccctggtctcgttttgctactaacacaactctaactaaggccaattcttacaatgtcgatttgagcaactcgactggtaccctagagtttgagttggtttacgctgttaacaccacacaaaccatatccaaatccgtctttcccgacttatcactttggttcaagggtttagaagatcctgaagaatatttaagaatgggttttgaagccagtgcttcttccttctttttggaccgtggtaactctaaggtcaagtttgtcaaggagaacccatatttcacaaacagaatgtctgtcaacaaccaaccattcaagtctgagaacgacctaagttactataaagtgtacggcctactggatcaaaacatcttggaattgtacttcaacgatggagatgtggtttctacaaatacctacttcatgaccaccggtaacgctctaggatctgtgaacatgaccactggtgtcgataatttgttctacattgacaagttccaagtaagggaagtaaaatagataacttcgtataatgtatgctatacgaagttataggatgtatatagataatgattgtttatgattagacattgattgagtgtagttggacattagcagtcagataggcaacgaagatcatccaagtctgaatacatacccatacaaatcatacaagtaaatgatggaattactcatataagtatgtacttacttgtaccgaattgccaatgaatgtcaatcagaacgcagtatgtacaagtactcgcacaatatcataaggcactcgaatgttcaagaagtcatcattttggtgattcggggaaatacttgacacctttgttgatgcaacttgactccataagtaggaaacccatagtatatctttttgtcgctttatattcacctgttcccttctttctatggactataagttaatttagttgaccttgtcaagtaatacctcaacaaactgtaaagaaataggagattattgcttttggtttttgagaagagatctggatagcaccacacaaataatgcgtcaaaatcagttcaaaatccaacccacagaacaaccaacactccccgaaccgctcataacctgtataggatgctgctccactcacacctcttgtccagccctaacctgcgatctaggttggggaaattttgtatgcaggaaaaatatatgcaagatttgggattttatttgtgttcatcaaccacgtcgatttacacagctatcatggtccgagaacgatctggaacggtggcgtacacgcccaaaaagcagcagaagcctctggtggacgacgcccacttgtcctctgctgaggaggacgatttcaagaccccagagagtgcaaagagcaagaagcctgaagcgtcacaacaggagccggcggtgtcagaaacgccagtcaagggcaaggccaagaagatcacctttgacgaggacggaatgagtgccgagcccatcgttgagaagaaaaaggtggttgtggaggagtctgaagacgacagtgacgatgcccccgaggaggaaacactggaggatggacaggaaaagaccctggccaagcaaaa

SEQ ID NO: 58 - DNA, 25 bp, Artificial Sequence
  Synthetic
accagatggtgtaacctccatcgac

SEQ ID NO: 59 - DNA, 25 bp, Artificial Sequence
  Synthetic
ggaagtggtggtctgggtatcgcag

SEQ ID NO: 60 - DNA, 27 bp, Artificial Sequence
  Synthetic
cacatacaccacaacacacacaaaatc

SEQ ID NO: 61 - DNA, 26 bp, Artificial Sequence
  Synthetic
ttcctctgagacaatcgcgtcggatc

SEQ ID NO: 62 - DNA, 3647 bp, Artificial Sequence
  Synthetic
acttcaagcacatccccaagtttatcaccgtcatctttctgctgctcaacgtggttgccatgatcgccggatccttcatggtcaccgccaagaagcgaattgaggtcggttgcggtctgctggtcggagtcattgtcacccaggctctggcctacggcctcatctttgactttggcttcattctgcgaaacctgtccgtcattggcggtcttttcatcgctcttaacgacgcctttgtcaaggacaagtccaagcgaggtctccctggtctcccctctattgacgacaaggaccggtccaagtacgtgctcctggcaggccgaatcctcctggttgtcatgttcacctccttcatcctcaatatgacctggactatgtctcgagttctggtgtccattgtcggaattgctgcttgctccatggtcgttgttggcttcaaggcccgagtttccgcgttcctcctgtgcatcatcctcttcatctttaacatcaccgccaactcctactgggccttccccgcctcttctcccgtccgagactacctcaagtacgagcacttccagaccctgtccatcattggcggtcttctgctggttgtcaacaccggagccggcaaaatctccattgacgagaagaagaaggtctactaagctattactagtcagcaacaacactttggtagagtttgatacagacattgacatctaccgcgatgtaaaaataatgtatactaggagagtctgtcgttcgcgagtggcaatgtcatgagttacacctgattcaaatagtgaataataatataacagccacaaatgaaagatgtatccacgggtagatatggctttaattactgatgattgaatgattatctgagtgctacagttgtaacgagtacgagttattacctgctactattgtactcctaaatcaaagtactcgtacatgcaatgaattacttgtataacttcgtataatgtatgctatacgaagttatatgcttttgcaagctttccttttccttttggctggttttgcagccaaaatatctgcatcaatgacaaacgaaactagcgatagacctttggtccacttcacacccaacaagggctggatgaatgacccaaatgggttgtggtacgatgaaaaagatgccaaatggcatctgtactttcaatacaacccaaatgacaccgtatggggtacgccattgttttggggccatgctacttccgatgatttgactcattgggaagatgaacccattgctatcgctcccaagcgtaacgattcaggtgctttctctggctccatggtggttgattacaacaacacgagtgggtttttcaatgatactattgatccaagacaaagatgcgttgcgatttggacttataacactcctgaaagtgaagagcaatacattagctattctcttgatggtggttacacttttactgaataccaaaagaaccctgttttagctgccaactccactcaattcagagatccaaaggtgttctggtatgaaccttctcaaaaatggattatgacggctgccaaatcacaagactacaaaattgaaatttactcctctgatgacttgaagtcctggaagctagaatctgcatttgctaatgaaggtttcttaggctaccaatatgaatgtccaggtttgattgaagtcccaactgagcaagatccttccaaatcctattgggtcatgtttatttctatcaatccaggtgcacctgctggcggttccttcaaccaatattttgttggatccttcaatggtactcattttgaagcgtttgacaatcaatctagagtggtagattttggtaaggactactatgccttgcaaactttcttcaacacagacccaacgtacggttcagcattaggtattgcctgggcttcaaactgggagtacagtgcctttgtcccaactaacccatggagatcatccatgtctttggtccgcaagttttctttgaacactgaatatcaagctaatccagagactgaattgatcaatttgaaagccgaaccaatattgaacattagtaatgctggtccctggtctcgttttgctactaacacaactctaactaaggccaattcttacaatgtcgatttgagcaactcgactggtaccctagagtttgagttggtttacgctgttaacaccacacaaaccatatccaaatccgtctttcccgacttatcactttggttcaagggtttagaagatcctgaagaatatttaagaatgggttttgaagccagtgcttcttccttctttttggaccgtggtaactctaaggtcaagtttgtcaaggagaacccatatttcacaaacagaatgtctgtcaacaaccaaccattcaagtctgagaacgacctaagttactataaagtgtacggcctactggatcaaaacatcttggaattgtacttcaacgatggagatgtggtttctacaaatacctacttcatgaccaccggtaacgctctaggatctgtgaacatgaccactggtgtcgataatttgttctacattgacaagttccaagtaagggaagtaaaatagataacttcgtataatgtatgctatacgaagttatattgtccaatgcagctggtatgcgatgtgtatgcttaacagatatatacatacgcctgggttgctgttcgagggatttcaggagtgtaagttgggtttctgctggggggtgtgacgaattggcagtgtcagtacttgtactggtacgactgatgtgtatgtaagctcaatgagcagcgtgctcctcggtctactatatggcgacatatctcttcgcttctgtttaccctttacatacggtacagctcttacaatgacatttatccactggcgtcgatccataaaccacacgaaccctgttttgttagtcaccatgaccggggcgtgtcgtgttacgttccaccgtttcacctcagccggttacgattcaacttgccgcgtcattctgcgttgctagcggagcgagtacgagtaactagactttcgataagctgaatgacttcagggtgcatgagaggcgcagatcgatattttcggacttgttcttctagaagactggtttgagagcaatcgcggaagagttgggaaccgctggagagcagaatgggttagtacagagactatctgatctcccccgcttgtgtctagccccactttatactctatttggcagttgttgcttgttattcaagcacagcatgtctgctcgtatcactttggagacatccctaacctctaaacctttatagatgccttaccttctgagcttgcagagagcattccacagcgaacccagttgacattatcagttgacatcttacgctcacaccacattacgcatctcacacacaatcacacgcacaagtacacacgcaagcacatcatacaatggtcaagggaattctgaaacataagacgccggaggccgaggttgcccccaaccctgaaatcgaccgacagaaggtcctggaaaacacacgagcgaacgcccagttgtatagccaaaactcggaaaaaatcagacgagctagcacgggcaaggtccgagtcgacgaggcgtcgtcgggcaccacggagttcc

SEQ ID NO: 63 - DNA, 3707 bp, Artificial Sequence
  Synthetic
ttatgcgagtgagtgacgtcatggaaagacgggcatttcagcttagtaagcacgttcgggggaaatgcggagtagaaaatggaagaaacaagaaattgatgaaaaccacttttgaggatgattcccaacgatcccggaaaaacggaaaatctgcaaacttttcaaaaaacaccgttaaaactttccaatggctccaatggcttcccaaataaagacagaggtaaatccaccgtattagtcaccttaccctcgttctatcgagcccaaacacggcccatttagcttaggatcatggccgattgtgcggtacaggggaaaatattttccatactcacatccctacaactctaatgacccggcatgacgaacttggtaatcgatccggataataaagaggaaagatcaggagaatgaggacagtgaaattagtggtatgtgcggggtacaagggtcatttgcggatgctagaggagcgtggggtattccgattggctcgattgacccgtttcacgccccgatcaagcacttgttccacatgtttctatactctgcgtctgcggagacgtcattttgcatgtctgaagtgggtttggggtttgtatggctgtcgagtaaagagcgcatggagatttttgtaaaattctagaaaaaatacttgtagtttcctcgctcatcatagttatcatcctggactctcacgttccacccccccacgctcactactagcacttgctgtagtcccctctctcgtctatctaccgccctaatgccgactgcactatgcgatgtccagataaggtagcaatccacacttctaggtttgtgaggtggtgttgaggtaccgacttgattgagacgactgaaaggacctccagctacatctaaaatcccagaggacgtgcgaagaaggtctttaagcccctttgtcttctagcgaccaaacccaacccgtcaaggcgacatacatcacgtcgacctcgtcccattgctgaacctaaacccacgcactccttgtataacataacttcgtataatgtatgctatacgaagttatatgcttttgcaagctttccttttccttttggctggttttgcagccaaaatatctgcatcaatgacaaacgaaactagcgatagacctttggtccacttcacacccaacaagggctggatgaatgacccaaatgggttgtggtacgatgaaaaagatgccaaatggcatctgtactttcaatacaacccaaatgacaccgtatggggtacgccattgttttggggccatgctacttccgatgatttgactcattgggaagatgaacccattgctatcgctcccaagcgtaacgattcaggtgctttctctggctccatggtggttgattacaacaacacgagtgggtttttcaatgatactattgatccaagacaaagatgcgttgcgatttggacttataacactcctgaaagtgaagagcaatacattagctattctcttgatggtggttacacttttactgaataccaaaagaaccctgttttagctgccaactccactcaattcagagatccaaaggtgttctggtatgaaccttctcaaaaatggattatgacggctgccaaatcacaagactacaaaattgaaatttactcctctgatgacttgaagtcctggaagctagaatctgcatttgctaatgaaggtttcttaggctaccaatatgaatgtccaggtttgattgaagtcccaactgagcaagatccttccaaatcctattgggtcatgtttatttctatcaatccaggtgcacctgctggcggttccttcaaccaatattttgttggatccttcaatggtactcattttgaagcgtttgacaatcaatctagagtggtagattttggtaaggactactatgccttgcaaactttcttcaacacagacccaacgtacggttcagcattaggtattgcctgggcttcaaactgggagtacagtgcctttgtcccaactaacccatggagatcatccatgtctttggtccgcaagttttctttgaacactgaatatcaagctaatccagagactgaattgatcaatttgaaagccgaaccaatattgaacattagtaatgctggtccctggtctcgttttgctactaacacaactctaactaaggccaattcttacaatgtcgatttgagcaactcgactggtaccctagagtttgagttggtttacgctgttaacaccacacaaaccatatccaaatccgtctttcccgacttatcactttggttcaagggtttagaagatcctgaagaatatttaagaatgggttttgaagccagtgcttcttccttctttttggaccgtggtaactctaaggtcaagtttgtcaaggagaacccatatttcacaaacagaatgtctgtcaacaaccaaccattcaagtctgagaacgacctaagttactataaagtgtacggcctactggatcaaaacatcttggaattgtacttcaacgatggagatgtggtttctacaaatacctacttcatgaccaccggtaacgctctaggatctgtgaacatgaccactggtgtcgataatttgttctacattgacaagttccaagtaagggaagtaaaatagataacttcgtataatgtatgctatacgaagttatacgaataaattaaatctattgtattttactgaacgcgacatgtaccgtatatgctaagtagtagctgctgtgtaatggaaccgtacgctaaagtatcgatgcgccaagctggtacacgtgcaccggttgcgatctgagtgtgtgttgtttccgtggtaacgggtgtgaaaagggagcgtggttggaaatgggaatggggtttgataatttgtttgtgactgttgtgtggatttgtatcgagtgtttatgatcgatgacgttggtaaggggtatcgagaggagccagaattggacgtggcaaactatttgacaaacggacgcatgggaacggaacagagtgttgagtgtcgagtacaataattgagatgtagagctgaaagtgtttcagcttatcagcatctgttgtcgtggtagtttcctgattacaatgtatgtacggtaggtgatccccagtcgtgctacagcgtcacgatcgatggggcgcaacaccacacactagatcctaaagctcgtgagcatggatgtagtttctggagttggaatttcgcaatcccttttgcccaaacagatctcaactacagtcgcagctagtgagtgtgtgctattggcctgctgagtggcgtaaggagacgtcatgaaaagtcctcatattgaagcagtttaggggtgatcagggcagtcgagaagataaatgtgacaagcttggatgtatcaacttcatgattaactgttggggacagccactcgaatgtggaagttgctaatggactcactcgactcaggcagatgacattatgatagaaagtggggtagtttcagttggatgcactaccaagagagcattatgttctataagtggtgcttgcgaaacattccgtaaactccacggaagttctcagactttcaatgagtctatatctcaacttctccggtcccgatatcttgtttttgtattgaggtacaggtatcgcacaaaggcggttcctcggcaatacggggaacgtcattgacgcaaccatg

SEQ ID NO: 64 - DNA, 26 bp, Artificial Sequence
  Synthetic
aactgcctcctcttgagcaggccaag

SEQ ID NO: 65 - DNA, 26 bp, Artificial Sequence
  Synthetic
ggaacagcagcttgatcttgatgtgc

SEQ ID NO: 66 - DNA, 3809 bp, Artificial Sequence
  Synthetic
tcacgtgacagatcctgatggccccgcgccccgcgcccatgcagctcactcacggcttcccgactctgacggtatggtgcaaccccacattagactcaaccgaacgcttagcgagatattgagcattgttgactgagacaagagagacaggcaagggaggcgcaaaaatatgtcgaatgattgaaaaattgcccagtggggcaattggtactcagcggagtaagtttttacccagaatccattggaatatccgtgatttgaatggtttgaagatgtggatctggatgctcaggctatttgttatatgagatagtacgaatacgactctaagttgactataagtacgactatcgctgaaaaaattacaccattggatttgtctggctgagagctctctaatggtgtgtttatttttcacacaacttggatatttcggccaataaccgatggccatggataattatagtaggtacaactaattagcggttattgtacagaaaacaaagaaactaagcatctaaggtctcccaaacacttgtattaggtcagcagaagagcttctacgatgaccacgatgtcaacctctcaatcatgtcaacctctcagtcaactcagcacctatcctccttccatttttcccattttcatcattttttcatttccgagagtatccgaaaatttgtcatgcatattgctcctgtttttcccgccgcagccatgcctttggcgaatggactactttccgccccgtatcttagtaatcacccgttgtgtcgtcttagtcatccagcaactgagagatataattgaaaagccacacgccgttgcgcaaattcaatcgcctcaactctactgatctgattactaacccccccgtctctcgcaccctctatgaacatgcaccctgcaagccaaccttaaaaatgcacagtaactgtcaacctgcatatactgcccactatctccaaccaacctttcaaagtgttttcccggcttcttttgcgaccgttccaccgactccacttcacgcccccagctctacgaagcgccaacatacacacaataacttcgtataatgtatgctatacgaagttatatgcttttgcaagctttccttttccttttggctggttttgcagccaaaatatctgcatcaatgacaaacgaaactagcgatagacctttggtccacttcacacccaacaagggctggatgaatgacccaaatgggttgtggtacgatgaaaaagatgccaaatggcatctgtactttcaatacaacccaaatgacaccgtatggggtacgccattgttttggggccatgctacttccgatgatttgactcattgggaagatgaacccattgctatcgctcccaagcgtaacgattcaggtgctttctctggctccatggtggttgattacaacaacacgagtgggtttttcaatgatactattgatccaagacaaagatgcgttgcgatttggacttataacactcctgaaagtgaagagcaatacattagctattctcttgatggtggttacacttttactgaataccaaaagaaccctgttttagctgccaactccactcaattcagagatccaaaggtgttctggtatgaaccttctcaaaaatggattatgacggctgccaaatcacaagactacaaaattgaaatttactcctctgatgacttgaagtcctggaagctagaatctgcatttgctaatgaaggtttcttaggctaccaatatgaatgtccaggtttgattgaagtcccaactgagcaagatccttccaaatcctattgggtcatgtttatttctatcaatccaggtgcacctgctggcggttccttcaaccaatattttgttggatccttcaatggtactcattttgaagcgtttgacaatcaatctagagtggtagattttggtaaggactactatgccttgcaaactttcttcaacacagacccaacgtacggttcagcattaggtattgcctgggcttcaaactgggagtacagtgcctttgtcccaactaacccatggagatcatccatgtctttggtccgcaagttttctttgaacactgaatatcaagctaatccagagactgaattgatcaatttgaaagccgaaccaatattgaacattagtaatgctggtccctggtctcgttttgctactaacacaactctaactaaggccaattcttacaatgtcgatttgagcaactcgactggtaccctagagtttgagttggtttacgctgttaacaccacacaaaccatatccaaatccgtctttcccgacttatcactttggttcaagggtttagaagatcctgaagaatatttaagaatgggttttgaagccagtgcttcttccttctttttggaccgtggtaactctaaggtcaagtttgtcaaggagaacccatatttcacaaacagaatgtctgtcaacaaccaaccattcaagtctgagaacgacctaagttactataaagtgtacggcctactggatcaaaacatcttggaattgtacttcaacgatggagatgtggtttctacaaatacctacttcatgaccaccggtaacgctctaggatctgtgaacatgaccactggtgtcgataatttgttctacattgacaagttccaagtaagggaagtaaaatagataacttcgtataatgtatgctatacgaagttatctgaaacatagcaattgatagacaagatttcgtacacacattccacattctatggacacccccgctgttcaacaccatctttttattcaaattattcagcacttagcagcaactcattttttttctcagttgcgtctccgaaccatcttcccatgcatggcaataatgagcagtctggtggggagacgaccatcgtcgctagagaaagccacagcccgcttttttctgtccaactttccacgtgtaggcacactagcagaccgtttgagggcatacggcggcttaacatactcatacccgtcctcaggagtggtcatgggagtgtcgtactcagactcaagcttgatgctgatctcctcggcctcctcgagcggccggtcgtatgagtcctgctcgtcagacgcatcatggaacgattcattatcagatgagtctgaagatgcggccgactcgtcaatcgcagagttgccacactccacagaggtcgtggtgccttgaggtgatccatggtcgtcatctgagtcggccttgtcatcaaacttcagggcttcagttttgacagcaggtgactcggcttcattgacaggggtagtgggctttgctgggcaagccgggccagccgggccagccggatcagaatgcttagacggagccgcgttggatgcttcagcgaccaactcagtagggtcaacagtctccaccgcagcagtctccgactcagcagtgtctgaagccgacttcgaaacagcaacggaaccaaaagcgtctactgactccgcagatgacccagaagcagtcgcaggctcaggcgagtcagagggggtcttgggtgttagagctttagctgaggtagagggctcgtcagactcgacctggggttcagaaaactcgatctggggtttagaagtttcgacctggggttcagaagactcgacctggagtctagaagactcaacctgacgctcagaagactcaacctggggttcagaaaactcggccacctgacgctcaggagactcgatctggggttcagaagactcgaccaccgcgggcccagaagactcatcaatccgagcctct

SEQ ID NO: 67 - DNA, 26 bp, Artificial Sequence
  Synthetic
gactggatctttcgactcaacagctc

SEQ ID NO: 68 - DNA, 26 bp, Artificial Sequence
  Synthetic
ccaaagacacaatcacgtcattggcc

SEQ ID NO: 69 - DNA, 3900 bp, Artificial Sequence
  Synthetic
agcatccccatctacaacaaggatacatctctggagatcctgcaggtgctgacgtcgcacactaccaaccctaataccgccgtggactactacaagaacctccgcgactctctgggcttttcaggtctcgtttgtctcacggagcctctttctcgagtcgatccgtccactgcaacctccatggtccctaaacttcttgctctggttaacagtgaggtgcccctggagtatctcaccgagctcatgaagtacgctccggaccgggtgtttgaatttgcatgcgaaactcggcagaagaatactctgcaagacatgtctgtggagtcagtagaggcgtttgtggtggcgagtctcaacagcggcatcgccccttatttggtgttccggttcctcatgaacctgagatccgtggccacctcttgcatctccgtgtcggctctaaagacagtgctgctggaaatggaccccgaagacgccaagaaggagtaccgggagtttgtcgaagacatctccaggggaaacatttgcatcgacaagctgtgggtcgccaagcacttgcatcacgagacgtacggctgcgatctccagccgtactttgatcccgctcgtctggacgacatctccgccaagggcgagtatctgatccccaacatttttgacggtctctacgccggtgcagagatcccctgccattctgtgttttacaactacaccacgttcgacaaggacctgcgaactgagggaactggggtggaactgagcacccgcttcgacggcaagctcaaggaggagtacactaaaaagtacacggaagctttcacttgtaaatagtaatttatgcatttcttatgataggacaaggtagttagagctcctcgttcttgtacataggttgctatccctacttgtagttattggtagaggtgggttagacaaaacaaaaggatagcaatactcaccagtgaacctcattgaatactacaaatttatttttctggtttttcccccgttactaggaaatttccccttgcagaaatgttcgtcagcattcgacaccccatgcattaagtctgcgcgcgtgcatatatagcaacgcactcaaaccttggacttgtacttgtactctctcatcacctcgttataacttcgtataatgtatgctatacgaagttatatgcttttgcaagctttccttttccttttggctggttttgcagccaaaatatctgcatcaatgacaaacgaaactagcgatagacctttggtccacttcacacccaacaagggctggatgaatgacccaaatgggttgtggtacgatgaaaaagatgccaaatggcatctgtactttcaatacaacccaaatgacaccgtatggggtacgccattgttttggggccatgctacttccgatgatttgactcattgggaagatgaacccattgctatcgctcccaagcgtaacgattcaggtgctttctctggctccatggtggttgattacaacaacacgagtgggtttttcaatgatactattgatccaagacaaagatgcgttgcgatttggacttataacactcctgaaagtgaagagcaatacattagctattctcttgatggtggttacacttttactgaataccaaaagaaccctgttttagctgccaactccactcaattcagagatccaaaggtgttctggtatgaaccttctcaaaaatggattatgacggctgccaaatcacaagactacaaaattgaaatttactcctctgatgacttgaagtcctggaagctagaatctgcatttgctaatgaaggtttcttaggctaccaatatgaatgtccaggtttgattgaagtcccaactgagcaagatccttccaaatcctattgggtcatgtttatttctatcaatccaggtgcacctgctggcggttccttcaaccaatattttgttggatccttcaatggtactcattttgaagcgtttgacaatcaatctagagtggtagattttggtaaggactactatgccttgcaaactttcttcaacacagacccaacgtacggttcagcattaggtattgcctgggcttcaaactgggagtacagtgcctttgtcccaactaacccatggagatcatccatgtctttggtccgcaagttttctttgaacactgaatatcaagctaatccagagactgaattgatcaatttgaaagccgaaccaatattgaacattagtaatgctggtccctggtctcgttttgctactaacacaactctaactaaggccaattcttacaatgtcgatttgagcaactcgactggtaccctagagtttgagttggtttacgctgttaacaccacacaaaccatatccaaatccgtctttcccgacttatcactttggttcaagggtttagaagatcctgaagaatatttaagaatgggttttgaagccagtgcttcttccttctttttggaccgtggtaactctaaggtcaagtttgtcaaggagaacccatatttcacaaacagaatgtctgtcaacaaccaaccattcaagtctgagaacgacctaagttactataaagtgtacggcctactggatcaaaacatcttggaattgtacttcaacgatggagatgtggtttctacaaatacctacttcatgaccaccggtaacgctctaggatctgtgaacatgaccactggtgtcgataatttgttctacattgacaagttccaagtaagggaagtaaaatagataacttcgtataatgtatgctatacgaagttatagaaataactaactaccacaccaacacatgaaatagatggaaattacatacatggtaaactaaaccagcatggccaaaagaggagcaaggaccagagcacccaccttcatcttagtagcagagttggcctggggagtctcaggctcagggccgggggtgacaggggactcaggagtaggctcttcaggtctaggagcaggagtctcgggctcagggccgggggtcacggggacctcaggagtaggctcttcaggtctgggagcaggagtctcgggctcagggccgggggtgacgggggcctcaggagtaggctcttcaggtctgggagcaggagtctcgggctcagggccgggggtaacggggacctcaggagtaggctcaggcttaacgacaggctcctcaggcttggaaagcgtctcgggagtaggctcgggcttaacgatgggcttctcaggagtgggctcgggcttaacgatgggcttctcagaggtgggctctggcttaacaacaggcttctcaggagtgggctcgggcttaaccacgggcttctcaggagaaggctggggcttggtaaccagggtggtaggcttaggctggggcttaggagtagactcgactggaatatcggtggggtcacaagggagaacaacggtaatagtcttgttgtcaatcacagtgacgacagtggtggtgatatggttagtggggacaacggtggtgagagtcacaggggtctcagagcaagtgtccatctcgcagacagtgacggtctgggtaaccacggtggtgatattggatgtaatggtctcagtatcgcagccgtcatcgcaaacagtctcggtctgagtggcaacagggatgggggtagcaggtccacagtcgtcatcacaatcagtgacggtcttcgtaacaaccacacagccaccatcatcacactcggttacagtcacagttacaactgggatggaggtggcaggctcacagccgtcatcacagtcagtgatagtctgggtaataacgggttcggatgtgactgcaggggaagactgagaaatggaggaggtctctgcagtgacagttggctcagaggagg

SEQ ID NO: 70 - DNA, 837 bp, Yarrowia lipolytica
atgcctgcaccagcaacctacgctactggcttgacgccccttcccacccccgtccctaaggtatccaagaacatcatggagcgattctctctgaagggaaaggttgcctctatcaccggttcttcttctggaatcggattcgctgttgctgaggcatttgcccaggctggtgccgatgtcgcgatctggtacaactccaagccttccgatgagaaggctgagtatctgtccaagacatacggagtccgatctaaggcttacaaatgtgctgtgaccaacgccaagcaggtcgagaccactatccaaaccatcgaaaaggactttggaaagattgacatcttcatcgccaacgcgggtatcccatggactgctggtccaatgatcgatgtccctaacaacgaggagtgggacaaggttgttgacctggatctcaacggtgcctattattgcgccaagtacgccggccagatcttcaagaagcagggctacggctccttcatcttcaccgcctccatgtctggccatattgtcaatatcccccagatgcaggcctgctacaacgcagctaagtgtgctgtcctccatctgtcccgatctctggccgtggagtgggctggattcgctcgatgtaatacagtgtcccctggttacatggctaccgagatttctgacttcatcccacgagacacaaaggagaagtggtggcagctcatccccatgggccgagagggagatccttctgagcttgctggagcctatatttacctggcttcggatgcctcaacttataccactggtgcagacattctggttgatggcggctactgttgtccttga

SEQ ID NO: 71 - DNA, 771 bp, Yarrowia lipolytica
atgtctggaccttccaccctcgccacgggactgcaccctctccccacagagaccccaaagttccccaccaacatcatggaccgattctccctcaagggtaaggttgcctccgtcaccggctcctcgtcaggtatcggctactgcgtggccgaggcctacgcccaggccggtgccgacgtggccatctggtacaactcccaccccgccgacgcaaaggctgagcacctcgctaagacctacggcgtcaaggccaaggcctacaagtgccctgtcaccgacgccgccgccgtggagtccaccatccagcagatcgagaaggactttggcaccattgacatcttcgtcgccaacgctggtgtcccctggaccgccggccccatgatcgacgtgcccgacaacaaggagtgggacaaggtcatcaacctggatctcaacggtgcctactactgcgccaagtacgccggccagatcttcaagaagaagggcaagggatccttcatcttcaccgcctccatgtccggccacattgtcaacatcccccagatgcaggcctgctacaacgccgccaaggccgctctgctgcacctgtctcgatcgctggccgtcgagtgggccggctttgcccgatgcaacacagtctcccctggctacatggccaccgagatctccgactttgtccccaaggagaccaaggagaagtggtggcagctcattcccatgggccgagagggagacccctccgagctctacctacctctaccttgcctctga

SEQ ID NO: 72 - DNA, 1032 bp, Yarrowia lipolytica
atgtctctcttttcactcgccaagaaaaccgccgtcatcaccggaggaagtggtggtctgggtatcgcagctgccaagcagcttcttcgagccggagcctctgttgctctggtcgacaacaacctgcctcgaatccagcctgctgccgagcagcttctagagtggtacaagaccgccaacgaggctcatcataacgtccgaccaacccccatctatgcctctcctactggcacacacaaggtttctgaaacagaaacagaatcaacaactgggggcttgaacgagcactctccacacgatatcaccaagcctgacatctctctggatgcttctgcagactccagtcagtcgtctgttgcccacgacgctgctcgagcccacgaagctgcaggaatacctcctggaaagggcaagaactttccccagcaacgaatctctgcctgggcatgcgatgtatctgacgtccaccaggtctccgataccgtcaaggccattcgagagcaccacaagagccccctcgatattttggtcaactgtgccggattctgcgagaatatgactgcctttgattatcccaacccccaggtcaagcgactgctggacgtcaacctcatgggatcctacaacttcgctaccgaggtggccaagtcgcttgtcctggacgagtctcctggatctctgattctggttgcatccatgagtggctccattgtcaacgacccccagccccagaccccctacaacatgtccaaggcaggtgtcatccacatggccaagtctctggctgccgagtgggcccagtacaacatccgagtcaacactctgtctcccggctacattcttactcctctgacccgtcacatcatcgagactgacggagagctccgaaacgactgggagcgacgaattcctttccgacgaatggctgagcccgaggagtttggaggccctattgtcttcatggcttccgacgcctccagctacatgaccggccacgatctcattgtcgatggaggttacaccatctggtaa

SEQ ID NO: 73 - DNA, 876 bp, Yarrowia lipolytica
atgtccaactccgccaaagccgctgtcgtgccccccgcccccaccgccgaagatatcgcccgagccaacgccggatccaaggaagagcccgttttccaggctaagaactttctgtccaagttccgactcgatggcaaggtagccattgtgactggtggagctcgaggactcggattctccatggccgagggtctgtgttcggtcggcctcaagggcattgccattctggatgtgcagcaggacctgggtctggatgccattgagaagctgcacaaggcctacggagtgcaggcccagttctacaaggccgacgtccgagacgaggagtccgtcaacgagatcatcgaccgagttgtgcacgatctcgggtccgtcgacgttgtggtcaactccgccggtgttgctgaccttgttcacgcagctgagtaccccgcagacaagttccgacgagtcatcgacatcaaccttaacggatccttcttggtgacccaggccgccgcccgacacatgatcaagcagggcaccggcggaaccgtggtgttcatcgcctccatgtccggatccattgtcaactggccccagcctcagagcgcttacaacgcctccaaggctgccgtcaagcacctgtctaagtcgctggccgccgagtgggccgtccacaacatccgatgcaactccatctcgcctggatacatggataccgctcttaaccgagcctacaacactctgtttgaggagtggaaggaccgaacccccctcggccgactcggagaccccgacgagctcaccggcgcctgcatctacctggcttccgatgcctcttcgtacgtgaccggatccgacattatcattgatggtggttacactattatttaa

SEQ ID NO: 74 - DNA, 2085 bp, Yarrowia lipolytica
atggctccccaattttcaaagactgacgagactgccatcaacaccatccgaaccttggctattgatgctgtggccaaggctaactccggccaccccggtgcccccatgggtctggctcctgttgcccacgttctgtggaactactacatgaacttcacctcctccaaccccgagtggatcaaccgagaccgattcattctctccaacggacacgcctgcatgctgcactactccctgttgcacctgtttggctacgacatcactatcgatgatctcaagaacttccgacagctcaactccaagactcccggccaccccgaggctgagactcccggtatcgaggtcaccaccggtcccctgggtcagggtgtctccaacgctgttggtttcgccattgcccaggcccaccttggcgccacctacaacaagcctggctacgacatcatcaacaactacacttactgcatcttcggagatggttgcatgatggagggtgttgcctccgaggctatgtctcttgccggacatctgcagctcggtaacctcatcaccttctacgatgataaccacatttccattgacggtgacaccaacgtggccttcaccgaggacgtcagccagcgacttgaggcctacggatgggaggtcatctgggtcaaggacggtaacaacgatctggccggcatggctgctgccatcgagcaggccaagaagtccaaggacaagcccacttgtatccgactcaccaccatcattggttacggctctctgcagcagggtacccacggtgttcacggctctcctctcaagcctgatgatatcaagcagttcaaggagaaggttggcttcaaccccgaggagacctttgccgtccccaaggagaccactgatctctacgccaagactattgaccgaggcgccaacgccgagaaggagtggaacgagctcttcgccaagtacggtaaggagtatcccaaggagcactctgagatcatccgacgattcaagcgagagctgcccgagggatgggagaaggctctgcctacctacacccccgccgacaatgccgttgcttctcgaaagctgtccgagattgtcctcaccaagatccacgaggtcctccccgagcttgttggtggttccgccgatctgaccggctcaaacctgacccgatggaaggacgctgttgatttccagcctcctgtcacccaccttggtgactactccggccgatatatccgatacggtgttcgagagcacggcatgggcgctatcatgaacggtatgaacgcttacggaggtatcatcccctacggaggtactttccttaacttcgtctcctacgccgctggtgccgtccgactgtctgccctgtctggccaccacgttatctgggttgctacccatgactccattggtctgggtgaggatggccctacccatcagcccattgagactgtcgcctggctccgagccacccccaacctctctgtgtggcgacctgccgacggtaacgagacctccgctgcttactacaaggccatcaccaactaccacactccctctgtcctgtctctgacccgacagaacctgcctcagcttgagggctcttccatcgagaaggcctccaagggtggttaccagctcatctccgaggacaagggtgacatctaccttgtgtccactggttctgaggttgccatctgtgttgctgccgccaagctcctcaaggagaagaagggtatcactgccggtgtcatctctctgcccgactggttcaccttcgagcagcagtctctcgagtaccgaaagtctgttttccccgatggcatccccatgctttccgtcgaggtctactccgactttggctggtctcgatactctcaccagcagtttggtctggaccgattcggtgcttctgctcccttccagcaggtctacgatgcctttgagttcaatgccgagggtgtcgccaagcgagctgaggccaccattaactactacaagggccagactgtcaagtctcctattcagcgagccttcgaccccattgacgtcaacacccgacccggccacggtgtctaa

SEQ ID NO: 75 - DNA, 1044 bp, Yarrowia lipolytica
atgccccctttcaccctggcagacactacggcgatacaggtgctcatgtcggcgacacaggatacccgctcggacttcacgcacaagctggcgccgctgctgcacctgctgtacacccggtttgtgcagtgcaaccacatgaaccccatgtggatcaacggcgacaagattctcttttcgtgtggcgacatgacaacgatccagagtcacgtgttgcgctactccgggtacaacgtcgacgaccgggagcttgaactgagcaaatatggagctgaggcgtttttcaatcagtttcaggagagcggaaatggctacaaacccaaggtggtggtgggcgataccgggtctgggtttgtggaggccgtgggagtggctctggacacgcaggagcttgcggagaacttcaacaagccgaatttccctctgatcacggcaaaaacatgggtcgtgttcgacgaggaggctgctctatcgtcggacgcaactaaggccgcagaagcagctgtcgaagcggaactcaataatctgattggtattcttgtggctgcatctccccaaacagtgcggttttaccatatgatgggctggcggctgctggaggttgtggatctcagtgatctggcgcaactcgaggcagtcatcgtggaggcgcttcaggagcctcacatgcctgtggttgtgcatatcagaagcattgagcggtccttggagagtgatgtgagcgataacacgctggtggacgagtatcatagatggggcgatgtgccggttgagagtgaccagagcgttaccacaagtctgtatacccgttttgccattatcaacgcgtcgcgtgagcttgcatggaatcatctgagggaagggtacaaggcattctttcctgcagattcggctgctctggaggaggttaagagggagcttgaggagtgttattatgatgagagggaacaagagggagggtcaaaggaggggctgacggtgcctctggagaggagtagtcggagtactcctagtaggacgccaaggctctgtgccgggttatag

SEQ ID NO: 76 - DNA, 1623 bp, Yarrowia lipolytica
atgtatctcggactggatctttcgactcaacagctcaagggcatcattctggacacaaaaacgctggacacggtcacacaagtccatgtggactttgaggacgacttgccgcagttcaacaccgaaaagggcgtctttcacagctctacagtggccggagaaatcaatgctcctgtggcaatgtggggggcagctgtggacttgctgatagagcgtctgtcaaaggaaatagacctttccacgatcaagtttgtgtcgggctcgtgccagcaacacggctctgtttatctcaacagcagctacaaggagggcctgggttctctggacaaacacaaagacttgtctacaggagtgtcatccttactggcgctcgaagtcagccccaattggcaggatgcaagcacggagaaggagtgtgcgcagtttgaggctgcagtcggcggtcccgagcagctggctgagatcactggctctcgagcacatactcgtttcaccgggccccagattctcaaggtcaaggaacgcaaccccaaggtattcaaggccacgtcacgggtccagctcatatccaactttctagcatctctgtttgccggcaaggcgtgcccctttgatcttgctgacgcctgtggaatgaatctgtgggacatccagaatggccagtggtgcaagaaactcacagatctcatcaccgatgacacccactcggtcgagtccctccttggagacgtggaaacagaccccaaggctctactgggcaaaatctcgccctatttcgtctccaagggcttctctccctcttgtcaggtggcacagttcacaggcgacaacccaggcactatgctggctctccccttacaggccaatgacgtgattgtgtctttgggaacatctacgaccgccctcgtcgtaacaaacaagtacatgcccgaccccggataccatgtgttcaaccaccccatggagggatacatgggcatgctgtgctactgcaacggaggtctagcacgagagaagatccgagacgagcttggaggctgggacgagtttaatgaggcggccgagaccaccaacacagtgtctgctgacgatgtccatgttggcatctactttccactacgagaaatccttcctcgagcaggtccctttgaacgacgtttcatctacaacagacaaagtgaacagcttacagagatggcttctccagaggactcactggcaaccgaacacaaaccgcaggctcaaaatctcaaggacacgtggccgccacaaatggacgccactgccatcattcaaagccaggccctcagtatcaaaatgagactccaacgcatgatgcatggcgatattggaaaggtgtattttgtgggaggcgcctcggtcaacactgctatctgcagcgtaatgtctgccatcttaaaaccaacaaagggcgcttggagatgtggtctggaaatggcaaacgcttgtgccattggaagtgcccatcacgcctggctttgcgaccccaacaagacaggccaggtacaggttcacgaagaagaggtcaaatacaagaatgtggacacagacgtgctactcaaggcgttcaagctggccgaaaacgcctgcctggagaaataa

SEQ ID NO: 77 - DNA, 732 bp, Yarrowia lipolytica
atgtcctccgaactgcctcctcttgagcaggccaagcgaatcgccgcccaccaggccgtggagcaacactaccccaaggacgccaaggtcgtgggcattggctcaggatccaccgtggtctacgttgccgagaagattgcgtcgttgcccaaggagctcaccaaggacaccgtgttcatttctacgggtttccagagcaagcagctgatccagaacgccgggttgcgactgggatgcatcgaccagtactccaacggagatctggacgtggcgtttgacggcgccgacgagaccgaccctcagctcaactgcatcaagggcggaggagcatgcctcttccaggaaaagattgtcgccgagtgtgcccgcaagtttgtcgtggtggccgactaccgaaaacagtccaaggctctgggcaccgtgtggatccagggtatccccattgaggtggtgcccgacgcctacaacaaggtaattgctgatctcaagaagatgggcgcccagtccgcggtgctgcgacccggctctcccggaaaggcgggccccatcatcaccgacaatggcaacttcattgtcgacgcctactttggcgagatccaacccgacgccgtcaaggacctgcacatcaagatcaagctgctgttgggcgtcgttgagaccggcctcttcactaacgcggacgtagcgtactttggaaacgccgacggaaccatctccaccattaccaagtaa

SEQ ID NO: 78 - DNA, 41 bp, Yarrowia lipolytica
ctgcagactaaatttatttcagtctcctcttcaccaccaaa

SEQ ID NO: 79 - DNA, 2126 bp, Yarrowia lipolytica
ctgcagactaaatttatttcagtctcctcttcaccaccaaaatggctccccaattttcaaagactgacgagactgccatcaacaccatccgaaccttggctattgatgctgtggccaaggctaactccggccaccccggtgcccccatgggtctggctcctgttgcccacgttctgtggaactactacatgaacttcacctcctccaaccccgagtggatcaaccgagaccgattcattctctccaacggacacgcctgcatgctgcactactccctgttgcacctgtttggctacgacatcactatcgatgatctcaagaacttccgacagctcaactccaagactcccggccaccccgaggctgagactcccggtatcgaggtcaccaccggtcccctgggtcagggtgtctccaacgctgttggtttcgccattgcccaggcccaccttggcgccacctacaacaagcctggctacgacatcatcaacaactacacttactgcatcttcggagatggttgcatgatggagggtgttgcctccgaggctatgtctcttgccggacatctgcagctcggtaacctcatcaccttctacgatgataaccacatttccattgacggtgacaccaacgtggccttcaccgaggacgtcagccagcgacttgaggcctacggatgggaggtcatctgggtcaaggacggtaacaacgatctggccggcatggctgctgccatcgagcaggccaagaagtccaaggacaagcccacttgtatccgactcaccaccatcattggttacggctctctgcagcagggtacccacggtgttcacggctctcctctcaagcctgatgatatcaagcagttcaaggagaaggttggcttcaaccccgaggagacctttgccgtccccaaggagaccactgatctctacgccaagactattgaccgaggcgccaacgccgagaaggagtggaacgagctcttcgccaagtacggtaaggagtatcccaaggagcactctgagatcatccgacgattcaagcgagagctgcccgagggatgggagaaggctctgcctacctacacccccgccgacaatgccgttgcttctcgaaagctgtccgagattgtcctcaccaagatccacgaggtcctccccgagcttgttggtggttccgccgatctgaccggctcaaacctgacccgatggaaggacgctgttgatttccagcctcctgtcacccaccttggtgactactccggccgatatatccgatacggtgttcgagagcacggcatgggcgctatcatgaacggtatgaacgcttacggaggtatcatcccctacggaggtactttccttaacttcgtctcctacgccgctggtgccgtccgactgtctgccctgtctggccaccacgttatctgggttgctacccatgactccattggtctgggtgaggatggccctacccatcagcccattgagactgtcgcctggctccgagccacccccaacctctctgtgtggcgacctgccgacggtaacgagacctccgctgcttactacaaggccatcaccaactaccacactccctctgtcctgtctctgacccgacagaacctgcctcagcttgagggctcttccatcgagaaggcctccaagggtggttaccagctcatctccgaggacaagggtgacatctaccttgtgtccactggttctgaggttgccatctgtgttgctgccgccaagctcctcaaggagaagaagggtatcactgccggtgtcatctctctgcccgactggttcaccttcgagcagcagtctctcgagtaccgaaagtctgttttccccgatggcatccccatgctttccgtcgaggtctactccgactttggctggtctcgatactctcaccagcagtttggtctggaccgattcggtgcttctgctcccttccagcaggtctacgatgcctttgagttcaatgccgagggtgtcgccaagcgagctgaggccaccattaactactacaagggccagactgtcaagtctcctattcagcgagccttcgaccccattgacgtcaacacccgacccggccacggtgtctaa